(19) United States
(12) Patent Application Publication
(10) Pub. No.: US 2025/0228842 A1
(43) Pub. Date: Jul. 17, 2025

(54) USES OF A SOMATOSTATIN MODULATOR FOR THE TREATMENT OF CARCINOID SYNDROME

(71) Applicant: Crinetics Pharmaceuticals, Inc., San Diego, CA (US)

(72) Inventors: Alan S. KRASNER, San Diego, CA (US); Hjalmar LAGAST, San Diego, CA (US); Keith S. USISKIN, San Diego, CA (US)

(21) Appl. No.: 18/727,827

(22) PCT Filed: Jan. 10, 2023
(86) PCT No.: PCT/US2023/010493

§ 371 Date: Jul. 10, 2024

Related U.S. Application Data
(60) Provisional application No. 63/298,551, filed on Jan. 11, 2022.

(51) Int. Cl. A61K 31/4709; A61K 9/20; A61K 9/48; A61P 35/00

(52) U.S. Cl. CPC A61K 31/4709; A61K 9/2009; A61K 9/2013; A61K 9/2018; A61K 9/2027; A61K 9/2054; A61K 9/2059; A61K 9/4825; A61K 9/485; A61K 9/4858; A61K 9/4866; A61P 35/00

(57) ABSTRACT

Described herein are uses of the somatostatin modulator 3-[4-(4-amino-piperidin-1-yl)-3-(3,5-difluoro-phenyl)-quinolin-6-yl]-2-hydroxy-benzonitrile, or a pharmaceutically acceptable salt thereof, in the treatment of carcinoid syndrome.

### CROSS-REFERENCE

This application claims benefit of U.S. Provisional Patent Application No. 63/298,551 filed on Jan. 11, 2022, which is incorporated herein by reference in its entirety.

### FIELD OF THE INVENTION

Described herein are pharmaceutical compositions and medicaments comprising a somatostatin modulator for use in the treatment of carcinoid syndrome.

### BACKGROUND OF THE INVENTION

Somatostatin is a peptide hormone that regulates the endocrine system and affects neurotransmission and cell proliferation via interaction with G-protein-coupled somatostatin receptors and inhibition of the release of numerous secondary hormones. Six subtype somatostatin receptor proteins have been identified (SSTR1, SSTR2a, SSTR2b, SSTR3, SSTR4, SSTR5) and are encoded by five different somatostatin receptor genes. Modulation of a particular subtype somatostatin receptor, or combination thereof, is attractive for the treatment of conditions, diseases, or disorders that would benefit from modulating somatostatin activity. Carcinoid syndrome is usually caused by well-differentiated NETs, nearly 90% of which express somatostatin receptors (SSTRs).

### SUMMARY OF THE INVENTION

In one aspect, described herein is the use of 3-[4-(4-amino-piperidin-1-yl)-3-(3,5-difluoro-phenyl)-quinolin-6-yl]-2-hydroxy-benzonitrile (Compound A), or a pharmaceutically acceptable salt (e.g., Compound A-monohydrochloride), or solvate thereof, in the treatment of carcinoid syndrome in a human. In some embodiments, Compound A, or a pharmaceutically acceptable salt (e.g., Compound A-monohydrochloride), or solvate thereof, is orally administered. In some embodiments, Compound A, or a pharmaceutically acceptable salt (e.g., Compound A-monohydrochloride), or solvate thereof, treats the symptoms of carcinoid syndrome in the human.

In one aspect, described herein is a method of treating carcinoid syndrome in a human comprising orally administering to the human with carcinoid syndrome a daily dose of 3-[4-(4-amino-piperidin-1-yl)-3-(3,5-difluoro-phenyl)-quinolin-6-yl]-2-hydroxy-benzonitrile (Compound A), or a pharmaceutically acceptable salt, or solvate thereof, sufficient to achieve a trough blood plasma concentration of Compound A of at least about 20 ng/ml, at least about 25 ng/mL, at least about 30 ng/ml, at least about 35 ng/mL, at least about 40 ng/ml, at least about 45 ng/ml, at least about 50 ng/ml, at least about 55 ng/ml, or at least about 60 ng/ml. The method of claim 1, wherein the trough blood plasma concentration of Compound A is at least 20 ng/ml, at least 21 ng/ml, at least 22 ng/mL, at least 23 ng/ml, at least 24 ng/ml, at least 25 ng/ml, at least 26 ng/ml, at least 27 ng/mL, at least 28 ng/mL, at least 29 ng/mL, at least 30 ng/mL, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/mL, at least 43 ng/ml, at least 44 ng/ml, at least 45 ng/ml, at least 46 ng/ml, at least 47 ng/mL, at least 48 ng/ml, at least 49 ng/mL, at least 50 ng/mL, at least 51 ng/mL, at least 52 ng/mL, at least 53 ng/mL, at least 54 ng/ml, at least 55 ng/ml, at least 56 ng/ml, at least 57 ng/mL, at least 58 ng/ml, at least 59 ng/mL, or at least 60 ng/ml. In some embodiments, the daily dose of Compound A, or a pharmaceutically acceptable salt, or solvate thereof is equivalent to about 40 mg/day, about 50 mg/day, about 60 mg/day, about 70 mg/day, about 80 mg/day, about 90 mg/day, about 100 mg/day, about 110 mg/day, or about 120 mg/day of Compound A-monohydrochloride. In some embodiments, the daily dose of Compound A, or a pharmaceutically acceptable salt, or solvate thereof is equivalent to about 40 mg/day, about 80 mg/day, or about 120 mg/day of Compound A-monohydrochloride.

In another aspect, described herein is a method of treating carcinoid syndrome in a human with carcinoid syndrome comprising: orally administering to the human with carcinoid syndrome an initial daily dose of 3-[4-(4-amino-piperidin-1-yl)-3-(3,5-difluoro-phenyl)-quinolin-6-yl]-2-hydroxy-benzonitrile (Compound A), or a pharmaceutically acceptable salt, or solvate thereof; determining the blood plasma trough concentration of Compound A in the human; and increasing the daily dose of Compound A, or a pharmaceutically acceptable salt, or solvate thereof if the human does not have at least a threshold trough blood plasma concentration of Compound A. In some embodiments, the threshold trough blood plasma concentration of Compound A is at least 20 ng/ml, at least 21 ng/ml, at least 22 ng/ml, at least 23 ng/ml, at least 24 ng/mL, at least 25 ng/mL, at least 26 ng/ml, at least 27 ng/mL, at least 28 ng/ml, at least 29 ng/ml, at least 30 ng/ml, at least 31 ng/ml, at least 32 ng/ml, at least 33 ng/ml, at least 34 ng/ml, at least 35 ng/mL, at least 36 ng/mL, at least 37 ng/mL, at least 38 ng/ml, at least 39 ng/ml, at least 40 ng/mL, at least 41 ng/mL, at least 42 ng/ml, at least 43 ng/mL, at least 44 ng/mL, at least 45 ng/ml, at least 46 ng/ml, at least 47 ng/ml, at least 48 ng/ml, at least 49 ng/ml, at least 50 ng/mL, at least 51 ng/mL, at least 52 ng/ml, at least 53 ng/mL, at least 54 ng/ml, at least 55 ng/mL, at least 56 ng/ml, at least 57 ng/ml, at least 58 ng/ml, at least 59 ng/mL, or at least 60 ng/ml. In some embodiments, the initial daily dose of Compound A, or a pharmaceutically acceptable salt, or solvate thereof comprises an amount equivalent to about 40 mg/day, about 50 mg/day, about 60 mg/day, about 70 mg/day, about 80 mg/day, about 90 mg/day, about 100 mg/day, about 110 mg/day, or about 120 mg/day of Compound A-monohydrochloride. In some embodiments, increasing the daily dose of Compound A, or a pharmaceutically acceptable salt, or solvate thereof comprises increasing the daily dose by an amount equivalent to about 10 mg/day, about 20 mg/day, about 30 mg/day, about 40 mg/day, about 50 mg/day, about 60 mg/day, about 70 mg/day, or about 80 mg/day of Compound A-monohydrochloride.

In another aspect, described herein is a method of treating carcinoid syndrome in a human with carcinoid syndrome comprising: orally administering to the human with carcinoid syndrome an initial daily dose of 3-[4-(4-amino-piperidin-1-yl)-3-(3,5-difluoro-phenyl)-quinolin-6-yl]-2-hydroxy-benzonitrile (Compound A), or a pharmaceutically acceptable salt, or solvate thereof that is equivalent to about 40 mg or about 80 mg of Compound A-monohydrochloride for an initial period of time; determining if symptoms of carcinoid syndrome decreased in frequency during the initial period of time; if symptoms of carcinoid syndrome decreased in frequency during the initial period of time then the daily amount of Compound A, or a pharmaceutically acceptable salt, or solvate thereof, that was administered during the initial period of time is continued at the same daily amount of Compound A, or a pharmaceutically acceptable salt, or solvate thereof, or if symptoms of carcinoid syndrome have not decreased in frequency during the initial period of time then the daily amount of Compound A, or a pharmaceutically acceptable salt, or solvate thereof, that was administered during the initial period of time is increased by an increment daily amount of Compound A, or a pharmaceutically acceptable salt, or solvate thereof; and the increased daily amount of Compound A, or a pharmaceutically acceptable salt, or solvate thereof, is administered to the human thereafter.

In some embodiments, the symptoms of carcinoid syndrome comprise the severity of diarrhea, frequency and intensity of flushing episodes, or combinations thereof. In some embodiments, the severity of diarrhea comprises the number of bowel movements per day, the number of watery stools as measured by Bristol Stool Scale, or both. In some embodiments, the initial daily dose of Compound A, or a pharmaceutically acceptable salt, or solvate thereof, is increased by an incremental daily dose that is equivalent to about 10 mg/day, about 20 mg/day, about 30 mg/day, or about 40 mg/day. In some embodiments, the initial daily dose of Compound A, or a pharmaceutically acceptable salt, or solvate thereof, is increased by an incremental daily dose that is equivalent to about 40 mg/day. In some embodiments, the increased daily amount of Compound A, or a pharmaceutically acceptable salt, or solvate thereof, is equivalent to about 80 mg or about 120 mg of Compound A-monohydrochloride.

In another aspect, described herein is a method of treating carcinoid syndrome in a human with carcinoid syndrome comprising orally administering to the human with carcinoid syndrome a pharmaceutical composition comprising 3-[4-(4-amino-piperidin-1-yl)-3-(3,5-difluoro-phenyl)-quinolin-6-yl]-2-hydroxy-benzonitrile (Compound A), or a pharmaceutically acceptable salt, or solvate thereof; wherein the human with carcinoid syndrome is treatment naïve; or wherein the human with carcinoid syndrome was previously treated with a somatostatin analog and the treatment with the somatostatin analog is terminated and a sufficient period of time lapses to allow the somatostatin analog to washout of the human; and wherein treatment is initiated at a daily dose equivalent to at least about 40 mg/day of Compound A-monohydrochloride.

In some embodiments, the human with carcinoid syndrome is treatment naïve to somatostatin analog therapy and actively symptomatic. In some embodiments, actively symptomatic comprises an average of ≥4 bowel movements (BM)/day or >2 flushing episodes in at least 2 days over a period of 2 weeks. In some embodiments, the carcinoid syndrome in the human that was previously treated with a somatostatin analog was symptomatically controlled with the somatostatin analog. In some embodiments, symptomatically controlled comprises an average of <4 bowel movements (BM)/day with ≤5 BMs on any single day and average ≤2 flushing episodes/day over a 2-week period. In some embodiments, the somatostatin analog is octreotide, lanreotide, or pasireotide. In some embodiments, treatment is initiated at a daily dose equivalent to about 40 mg/day or 80 mg/day of Compound A-monohydrochloride. In some embodiments, the daily dose is increased by a daily dose amount equivalent to about 40 mg/day Compound A-monohydrochloride if breakthrough symptoms are observed. In some embodiments, the breakthrough symptoms comprise ≥4 bowel movements (BMs)/day for 2 consecutive days or flushing frequency ≥3 flushing episodes/day for at least 1 day.

In another aspect, described herein is a method of treating carcinoid syndrome in a human with carcinoid syndrome comprising orally administering to the human with carcinoid syndrome a daily dose of 3-[4-(4-amino-piperidin-1-yl)-3-(3,5-difluoro-phenyl)-quinolin-6-yl]-2-hydroxy-benzonitrile (Compound A), or a pharmaceutically acceptable salt, or solvate thereof, wherein treating carcinoid syndrome comprises managing the symptoms of the carcinoid syndrome.

In some embodiments, managing the symptoms of the carcinoid syndrome comprises reducing the frequency of: daily bowel movements, episodes of diarrhea, episodes of fecal incontinence, cutaneous flushing, or a combination thereof. In some embodiments, managing the symptoms of the carcinoid syndrome comprises treating diarrhea, flushing episodes, or both in the human. In some embodiments, the diarrhea is severe diarrhea. In some embodiments, managing the symptoms of the carcinoid syndrome comprises reducing the number of bowel movements (BM) to less than 4 BM/day for 2 consecutive days, reducing the flushing frequency to less than 3 flushing episodes/day for at least 1 day, or both.

In some embodiments, the daily dose of Compound A, or a pharmaceutically acceptable salt, or solvate thereof, is equivalent to about 40 mg or about 80 mg of Compound A-monohydrochloride. In some embodiments, the daily dose of Compound A, or a pharmaceutically acceptable salt, or solvate thereof, is increased by an incremental daily dose that is equivalent to about 10 mg/day, about 20 mg/day, about 30 mg/day, or about 40 mg/day if the severity of the diarrhea, flushing episodes or both does not diminish in severity during treatment with the daily dose of Compound A, or a pharmaceutically acceptable salt, or solvate thereof. In some embodiments, the daily dose of Compound A, or a pharmaceutically acceptable salt, or solvate thereof, is increased by an incremental daily dose that is equivalent to about 40 mg/day. In some embodiments, the daily dose of Compound A, or a pharmaceutically acceptable salt, or solvate thereof, is increased by an incremental daily dose that is equivalent to about 40 mg/day and treatment is continued at a daily amount of Compound A, or a pharmaceutically acceptable salt, or solvate thereof, that is equivalent to about 80 mg or about 120 mg of Compound A-monohydrochloride.

In another aspect, described herein is a method of treating severe diarrhea, flushing episodes or both in a human with carcinoid syndrome comprising orally administering to the human in need thereof an initial daily dose of 3-[4-(4-amino-piperidin-1-yl)-3-(3,5-difluoro-phenyl)-quinolin-6-yl]-2-hydroxy-benzonitrile (Compound A), or a pharmaceutically acceptable salt, or solvate thereof. In some embodiments, the initial daily dose of Compound A, or a pharmaceutically acceptable salt, or solvate thereof, is equivalent to about 40 mg or about 80 mg of Compound A-monohydrochloride. In some embodiments, the initial daily dose of Compound A, or a pharmaceutically acceptable salt, or solvate thereof, is increased by an incremental daily dose that is equivalent to about 10 mg/day, about 20 mg/day, about 30 mg/day, or about 40 mg/day if the severity of the severe diarrhea, flushing episodes or both does not diminish in severity during treatment with the initial daily dose of Compound A, or a pharmaceutically acceptable salt, or solvate thereof. In some embodiments, the initial daily dose of Compound A, or a pharmaceutically acceptable salt, or solvate thereof, is increased by an incremental daily dose that is equivalent to about 40 mg/day. In some embodiments, the daily dose of Compound A, or a pharmaceutically acceptable salt, or solvate thereof, is increased by an incremental daily dose that is equivalent to about 40 mg/day and treatment is continued at a daily amount of Compound A, or a pharmaceutically acceptable salt, or solvate thereof, that is equivalent to about 80 mg or about 120 mg of Compound A-monohydrochloride

In some embodiments, the severity of the severe diarrhea is measured by the number of bowel movements per day, the number of watery stools as measured by Bristol Stool Scale, or both. In some embodiments, the severity of the flushing episodes is assessed by measuring urinary 5-hydroxyindole acetic acid (5-HIAA) levels, plasma serotonin levels, or both.

In another aspect, described herein is a method of treating carcinoid syndrome in a human with carcinoid syndrome comprising orally administering to the human with carcinoid syndrome a daily dose of 3-[4-(4-amino-piperidin-1-yl)-3-(3,5-difluoro-phenyl)-quinolin-6-yl]-2-hydroxy-benzonitrile (Compound A), or a pharmaceutically acceptable salt, or solvate thereof, wherein human with carcinoid syndrome was previously treated with a somatostatin analog; and wherein treatment is initiated at a daily dose equivalent to about 40 mg/day of Compound A-monohydrochloride.

In another aspect, described herein is a method of treating carcinoid syndrome in a human with carcinoid syndrome comprising orally administering to the human with carcinoid syndrome a daily dose of 3-[4-(4-amino-piperidin-1-yl)-3-(3,5-difluoro-phenyl)-quinolin-6-yl]-2-hydroxy-benzonitrile (Compound A), or a pharmaceutically acceptable salt, or solvate thereof, wherein human with carcinoid syndrome was previously treated with a somatostatin analog; and wherein treatment is initiated at a daily dose equivalent to about 80 mg/day of Compound A-monohydrochloride.

In some embodiments, the human with carcinoid syndrome responded to and tolerated treatment with a somatostatin analog. In some embodiments, carcinoid syndrome symptoms were previously controlled on octreotide or lanreotide depot monotherapy. In some embodiments, the human with carcinoid syndrome is refractory to treatment with somatostatin analog. In some embodiments, the somatostatin analog is octreotide, lanreotide, or pasireotide.

In some embodiments, treating carcinoid syndrome comprises managing the symptoms of the carcinoid syndrome. In some embodiments, managing the symptoms of the carcinoid syndrome comprises reducing the frequency of: daily bowel movements, episodes of diarrhea, episodes of fecal incontinence, cutaneous flushing, or a combination thereof. In some embodiments, managing the symptoms of the carcinoid syndrome comprises treating diarrhea, flushing episodes, or both in the human. In some embodiments, the diarrhea is severe diarrhea. In some embodiments, the severity of diarrhea comprises the number of bowel movements per day, the number of watery stools as measured by Bristol Stool Scale, or both. In some embodiments, managing the symptoms of the carcinoid syndrome comprises reducing the number of bowel movements (BM) to less than 4 BM/day for 2 consecutive days, reducing the flushing frequency to less than 3 flushing episodes/day for at least 1 day, or both.

In some embodiments, the daily dose of Compound A, or a pharmaceutically acceptable salt, or solvate thereof, is increased by an incremental daily dose that is equivalent to about 10 mg/day, about 20 mg/day, about 30 mg/day, or about 40 mg/day if the severity of the diarrhea, flushing episodes or both does not diminish in severity during treatment with the daily dose of Compound A, or a pharmaceutically acceptable salt, or solvate thereof.

In some embodiments, the daily dose of Compound A, or a pharmaceutically acceptable salt, or solvate thereof, is increased by an incremental daily dose that is equivalent to about 40 mg/day. In some embodiments, the daily dose of Compound A, or a pharmaceutically acceptable salt, or solvate thereof, is increased by an incremental daily dose that is equivalent to about 40 mg/day and treatment is continued at a daily amount of Compound A, or a pharmaceutically acceptable salt, or solvate thereof, that is equivalent to about 80 mg or about 120 mg of Compound A-monohydrochloride.

In some embodiments, the neuroendocrine tumors (NETs) in the human are grade 1 or grade 2. In some embodiments, the neuroendocrine tumors (NETs) in the human comprise positive SSTR tumor status.

In some embodiments, the serum 5-hydroxy-indoleacetic acid (5-HIAA) concentration in the human prior to treatment with Compound A is greater than about >280 μmol/L.

In some embodiments, treatment with Compound A, or a pharmaceutically acceptable salt, or solvate thereof, is discontinued if the human does not tolerate Compound A, or a pharmaceutically acceptable salt, or solvate thereof.

In some embodiments, the daily dose of Compound A, or a pharmaceutically acceptable salt, or solvate thereof, is reduced if the human does not tolerate Compound A, or a pharmaceutically acceptable salt, or solvate thereof. In some embodiments, the daily dose of Compound A, or a pharmaceutically acceptable salt, or solvate thereof, is reduced by an amount equivalent to about 40 mg of Compound A-monohydrochloride if the human does not tolerate Compound A, or a pharmaceutically acceptable salt, or solvate thereof

In some embodiments, Compound A, or a pharmaceutically acceptable salt, or solvate thereof, is administered in the form of one or more tablets comprising a spray-dried solid dispersion of 3-[4-(4-amino-piperidin-1-yl)-3-(3,5-difluoro-phenyl)-quinolin-6-yl]-2-hydroxy-benzonitrile monohydrochloride, or solvate thereof; one or more additional pharmaceutically acceptable ingredients; and optionally one or more film coating agents.

In some embodiments, the spray-dried solid dispersion comprises: (a) 3-[4-(4-amino-piperidin-1-yl)-3-(3,5-difluoro-phenyl)-quinolin-6-yl]-2-hydroxy-benzonitrile monohydrochloride, or solvate thereof; and (b) a pharmaceutically acceptable polymer; wherein 3-[4-(4-amino-piperidin-1-yl)-3-(3,5-difluoro-phenyl)-quinolin-6-yl]-2-hydroxy-benzonitrile monohydrochloride, or solvate thereof, is dispersed in a polymer matrix formed from the pharmaceutically acceptable polymer.

In some embodiments, the one or more pharmaceutically acceptable ingredients are selected from the group consisting of one or more diluents, one or more disintegrants, one or more lubricants, one or more glidants. In some embodiments, the one or more pharmaceutically acceptable ingredients comprise microcrystalline cellulose, mannitol, pregelatinized starch croscarmellose sodium crospovidone, sodium chloride, 1:1 sodium chloride:potassium chloride, colloidal silicon dioxide, and magnesium stearate.

In some embodiments, each tablet comprises about 2% by weight to about 20% by weight of 3-[4-(4-amino-piperidin-1-yl)-3-(3,5-difluoro-phenyl)-quinolin-6-yl]-2-hydroxy-benzonitrile monohydrochloride, or solvate thereof. In some embodiments, each tablet comprises about 2% by weight, about 3% by weight, about 4% by weight, about 5% by weight, about 6% by weight, about 7% by weight, about 8% by weight, about 9% by weight, about 10% by weight, about 11% by weight, about 12% by weight, about 13% by weight, about 14% by weight, or about 15% by weight of 3-[4-(4-amino-piperidin-1-yl)-3-(3,5-difluoro-phenyl)-quinolin-6-yl]-2-hydroxy-benzonitrile monohydrochloride, or solvate thereof.

In some embodiments, each tablet comprises about 10% by weight to about 35% by weight of the polymer matrix formed from the pharmaceutically acceptable polymer. In some embodiments, each tablet comprises: about 2% by weight to about 15% by weight of 3-[4-(4-amino-piperidin-1-yl)-3-(3,5-difluoro-phenyl)-quinolin-6-yl]-2-hydroxy-benzonitrile monohydrochloride, or solvate thereof, dispersed in a polymer matrix formed from a pharmaceutically acceptable polymer, wherein the dispersed 3-[4-(4-amino-piperidin-1-yl)-3-(3,5-difluoro-phenyl)-quinolin-6-yl]-2-hydroxy-benzonitrile monohydrochloride, or solvate thereof, in the polymer matrix is about 20% by weight to about 35% by weight of the tablet; about 40% by weight to about 80% by weight of one or more pharmaceutically acceptable ingredients selected from the group consisting of one or more diluents, one or more disintegrants, one or more lubricants, one or more glidants; and optionally less than about 5% by weight of one or more film coating agents. In some embodiments, the tablet comprises: about 20% by weight to about 40% of a spray dried dispersion of 3-[4-(4-amino-piperidin-1-yl)-3-(3,5-difluoro-phenyl)-quinolin-6-yl]-2-hydroxy-benzonitrile monohydrochloride, or solvate thereof, dispersed in a polymer matrix formed from a pharmaceutically acceptable polymer; about 60% by weight to about 80% by weight of one or more pharmaceutical acceptable ingredients selected from the group consisting of one or more diluents, one or more disintegrants, one or more disintegrant aids, one or more lubricants, one or more glidants; and optionally less than about 5% by weight of one or more film coating agents.

In some embodiments, the spray dried dispersion comprises an about 15/85 to about 35/65 ratio of 3-[4-(4-amino-piperidin-1-yl)-3-(3,5-difluoro-phenyl)-quinolin-6-yl]-2-hydroxy-benzonitrile monohydrochloride, or solvate thereof to a polymer matrix of hydroxypropyl methyl cellulose acetate succinate (HPMCAS), or polyvinylpyrrolidone polyvinyl acetate copolymers (PVP/VA).

In some embodiments, each tablet comprises: about 20% by weight to about 35% of by weight a spray dried dispersion of 3-[4-(4-amino-piperidin-1-yl)-3-(3,5-difluoro-phenyl)-quinolin-6-yl]-2-hydroxy-benzonitrile monohydrochloride, or solvate thereof, dispersed in a polymer matrix formed from a pharmaceutically acceptable polymer; wherein the spray dried dispersion comprises an about 15/85 to about 35/65 ratio of 3-[4-(4-amino-piperidin-1-yl)-3-(3,5-difluoro-phenyl)-quinolin-6-yl]-2-hydroxy-benzonitrile monohydrochloride, or solvate thereof to a polymer matrix of hydroxypropyl methyl cellulose acetate succinate (HPMCAS), or polyvinylpyrrolidone polyvinyl acetate copolymers (PVP/VA); about 60% by weight to about 80% by weight of one or more pharmaceutical acceptable ingredients selected from the group consisting of microcrystalline cellulose, mannitol, pregelatinized starch, croscarmellose sodium, crospovidone, sodium chloride, 1:1 sodium chloride:potassium chloride, silicon dioxide, and magnesium stearate; optionally less than about 5% by weight of one or more film coating agents.

In some embodiments, each tablet comprises: about 20%, about 21%, about 22%, about 23%, about 24%, about 25%, about 26%, about 27%, about 28%, about 29%, about 30%, about 31%, about 32%, about 33%, about 34%, or about 35% by weight of a spray dried dispersion of 3-[4-(4-amino-piperidin-1-yl)-3-(3,5-difluoro-phenyl)-quinolin-6-yl]-2-hydroxy-benzonitrile monohydrochloride, or solvate thereof, dispersed in a polymer matrix formed from a pharmaceutically acceptable polymer; wherein the spray dried dispersion comprises an about 15/85 or about 35/65 ratio of 3-[4-(4-amino-piperidin-1-yl)-3-(3,5-difluoro-phenyl)-quinolin-6-yl]-2-hydroxy-benzonitrile monohydrochloride, or solvate thereof to a polymer matrix of hydroxypropyl methyl cellulose acetate succinate (HPMCAS), or polyvinylpyrrolidone polyvinyl acetate copolymers (PVP/VA); about 60% by weight to about 80% by weight of one or more pharmaceutical acceptable ingredients selected from the group consisting of one or more diluents, one or more disintegrants, one or more disintegrant aids, one or more lubricants, one or more glidants; and optionally less than about 5% by weight of one or more film coating agents.

In some embodiments, each tablet comprises: about 20%, about 21%, about 22%, about 23%, about 24%, about 25%, about 26%, about 27%, about 28%, about 29%, about 30%, about 31%, about 32%, about 33%, about 34%, or about 35% by weight of a spray dried dispersion of 3-[4-(4-amino-piperidin-1-yl)-3-(3,5-difluoro-phenyl)-quinolin-6-yl]-2-hydroxy-benzonitrile monohydrochloride, or solvate thereof, dispersed in a polymer matrix formed from a pharmaceutically acceptable polymer; wherein the spray dried dispersion comprises an about 15/85 or about 35/65 ratio of 3-[4-(4-amino-piperidin-1-yl)-3-(3,5-difluoro-phenyl)-quinolin-6-yl]-2-hydroxy-benzonitrile monohydrochloride, or solvate thereof to a polymer matrix of hydroxypropyl methyl cellulose acetate succinate (HPMCAS), or polyvinylpyrrolidone polyvinyl acetate copolymers (PVP/VA); about 60% by weight to about 80% by weight of one or more pharmaceutical acceptable ingredients selected from the group consisting of microcrystalline cellulose, mannitol, pregelatinized starch, croscarmellose sodium, crospovidone, sodium chloride, 1:1 sodium chloride:potassium chloride, silicon dioxide, and magnesium stearate; optionally less than about 5% by weight of one or more film coating agents.

In some embodiments, each tablet comprises about 10 mg, about 20 mg, about 40 mg, about 60 mg or about 80 mg of 3-[4-(4-amino-piperidin-1-yl)-3-(3,5-difluoro-phenyl)-quinolin-6-yl]-2-hydroxy-benzonitrile monohydrochloride, or solvate thereof. In some embodiments, each tablet comprises about 10 mg of 3-[4-(4-amino-piperidin-1-yl)-3-(3,5-difluoro-phenyl)-quinolin-6-yl]-2-hydroxy-benzonitrile monohydrochloride, or solvate thereof. In some embodiments, each tablet comprises about 20 mg of 3-[4-(4-amino-piperidin-1-yl)-3-(3,5-difluoro-phenyl)-quinolin-6-yl]-2-hydroxy-benzonitrile monohydrochloride, or solvate thereof.

In some embodiments, the one or more tablets are administered at least 30 minutes before a meal. In some embodiments, the one or more tablets are administered at least 60 minutes before a meal. In some embodiments, the one or more tablets are administered with a glass of water on an empty stomach at least 30 minutes before a meal.

In some embodiments, the bioavailability of 3-[4-(4-amino-piperidin-1-yl)-3-(3,5-difluoro-phenyl)-quinolin-6-yl]-2-hydroxy-benzonitrile monohydrochloride, or solvate thereof, is not substantially affected by the coadministration of proton pump inhibitors, histamine H2-receptor antagonists, or antacids. In some embodiments, Compound A, or a pharmaceutically acceptable salt, or solvate thereof, is not co-administered with a drug that alters the pH of the upper gastrointestinal (GI) tract. In some embodiments, if Compound A, or a pharmaceutically acceptable salt, or solvate thereof, is co-administered with a drug that alters the pH of the upper gastrointestinal (GI) tract, then the daily dose amount of Compound A, or a pharmaceutically acceptable salt, or solvate thereof, that is administered is increased. In some embodiments, the daily dose amount of Compound A, or a pharmaceutically acceptable salt, or solvate thereof, is increased by an amount equivalent to about 10 mg/day, about 20 mg/day, about 30 mg/day, or about 40 mg/day of Compound A-monohydrochloride. In some embodiments, the drug that alters the pH of the upper gastrointestinal (GI) tract comprises proton pump inhibitors, histamine H2-receptor antagonists, or antacids.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE DRAWINGS

FIG. 1. Illustrates the observed dose proportionality in humans administered the HMG capsule formulation or the SDD tablet formulation of Compound A-HCl.

FIG. 2. Illustrates the performance of the HMG capsule formulation and the SDD tablet formulation of Compound A-HCl in dogs with or without pentagastrin pretreatment.

FIG. 3. Illustrates the hormone levels observed in primary analysis population of the Acrobat Edge clinical trial.

FIG. 4. Illustrates the evidence of a dose response observed in the Acrobat Edge and Evolve clinical trials.

FIG. 5. Illustrates the estimated trough plasma paltusotine concentrations needed for therapeutic effect and projections of the dose proportional effects projected for the SDD tablet formulations.

FIG. 6. Illustrates the projected trough plasma paltusotine concentrations of acromegaly patients taking PPIs who are on 60 mg SDD tablets (1 hour post dose fast) compared to projected trough plasma paltusotine concentrations of acromegaly patients not taking PPIs and trough plasma paltusotine concentrations of acromegaly patients who are on 40 mg HMG capsule formulations (2 h post dose fast).

### DETAILED DESCRIPTION OF THE INVENTION

Somatostatin (SST), also known as somatotropin release inhibiting factor (SRIF) was initially isolated as a 14-amino acid peptide from ovine hypothalamus (Brazeau et al., Science 179, 77-79, 1973). An N-terminal extended 28-amino acid peptide with similar biological activity to 14-amino acid somatostatin was subsequently isolated (Pradayrol et, al., FEBS Letters, 109, 55-58, 1980; Esch et al., Proc. Natl. Acad. Sci. USA, 77, 6827-6831, 1980). SST is a regulatory peptide produced by several cell types in response to other neuropeptides, neurotransmitters, hormones, cytokines, and growth factors. SST acts through both endocrine and paracrine pathways to affect its target cells. Many of these effects are related to the inhibition of secretion of other hormones, most notably growth hormone (GH). They are produced by a wide variety of cell types in the central nervous system (CNS) and gut, and have multiple functions including modulation of secretion of growth hormone (GH), insulin, glucagon, as well as many other hormones that are anti-proliferative.

These pleotropic actions of somatostatins are mediated by six somatostatin receptor proteins (SSTR1, SSTR2a, SSTR2b, SSTR3, SSTR4, SSTR5). The six somatostatin receptor proteins are encoded by five different somatostatin receptor genes (Reisine and Bell, Endocr Rev. 16, 427-442, 1995; Patel and Srikant, Trends Endocrinol Metab 8, 398-405, 1997). All the receptors are members of the class-A subgroup of the G protein-coupled receptor (GPCR) superfamily. SST2A receptor is the most widely expressed subtype in human tumors and is the dominant receptor by which GH secretion is suppressed. Unless otherwise stated, the term SSTR2 means SSTR2a.

It is possible to selectively modulate any one of the somatostatin receptor subtypes, or combination thereof. In some embodiments, selectively modulating any one of the somatostatin receptor subtypes relative to the other somatostatin receptor subtypes, or combination thereof, is useful in a variety of clinical applications. In some embodiments, selectively modulating any one of the somatostatin receptor subtypes relative to the other somatostatin receptor subtypes reduces unwanted side effects in a variety of clinical applications.

For example, modulation of SSTR2 activity mediates the inhibition of growth hormone (GH) release from the anterior pituitary and glucagon release from pancreas. SSTR2 is also implicated in many other biological functions such as, but not limited to, cell proliferation, nociception, inflammation, and angiogenesis. In some embodiments, a selective SSTR2 modulator is used in the treatment of acromegaly, gut neuroendocrine tumors, pain, neuropathies, nephropathies, and inflammation, as well as retinopathies resulting from aberrant blood vessel growth.

In some embodiments, SSTR3 agonists inhibit insulin secretion. In some embodiments, SSTR4 agonists exhibit anti-inflammatory and anti-nociceptive effects. In some embodiments, SSTR5 agonists inhibit insulin secretion. In addition, SSTR5 has also been implicated to modulate the release of growth hormone.

In some embodiments, the somatostatin modulator 3-[4-(4-amino-piperidin-1-yl)-3-(3,5-difluoro-phenyl)-quinolin-6-yl]-2-hydroxy-benzonitrile monohydrochloride (Compound A) has utility over a wide range of therapeutic applications. In some embodiments, the somatostatin receptor modulator described herein is used in the treatment of a variety of diseases or conditions such as, but not limited to acromegaly, neuroendocrine tumors, retinopathies and other ophthalmic disorders, neuropathy, nephropathy, respiratory diseases, cancers, pain, neurodegenerative diseases, inflammatory diseases, as well as psychiatric and neurodegenerative disorders. In some embodiments, the somatostatin receptor modulator described herein is used in the treatment of acromegaly, neuroendocrine tumors, or both in a mammal. In some embodiments, the somatostatin receptor modulator described herein is used in the treatment of acromegaly in a mammal. In some embodiments, the somatostatin receptor modulator described herein is used in the treatment of neuroendocrine tumors.

The compound 3-[4-(4-amino-piperidin-1-yl)-3-(3,5-difluoro-phenyl)-quinolin-6-yl]-2-hydroxy-benzonitrile monohydrochloride (Compound A), or solvate thereof, is a selective nonpeptide SST2 biased agonist that is amenable to oral administration to a mammal in need of treatment with a somatostatin modulator. In some embodiments, mammal in need of treatment with a somatostatin modulator has been diagnosed with neuroendocrine tumors (NETs). In some embodiments, mammal in need of treatment with a somatostatin modulator has been diagnosed with well-differentiated NETs. In some embodiments, mammal in need of treatment with a somatostatin modulator has been diagnosed with carcinoid syndrome.

Neuroendocrine tumors (NETs) are a heterogeneous group of tumors that arise from enterochromaffin cells with traits of both nervous system and hormone-producing cells. NETs are principally characterized by originating site, grade, and stage, as well as functional status. Most NETs are found in the gastrointestinal (GI) tract, but NETs may occur in other sites, including the lungs. Within the GI tract, the most common sites are the small bowel, rectum, and colon, followed by the pancreas, stomach, and appendix.

Well-differentiated NETs are commonly referred to as “carcinoid tumors,” a term in common use by clinicians and patients. The World Health Organization (WHO) classification system, which does not recognize “carcinoid tumor,” was developed to unify various approaches to tumor characterization and staging, thus enabling greater worldwide consistency in reporting.

Grading schemes for neuroendocrine tumors (NETs) use mitotic count; the level of the nuclear protein Ki-67, which is associated with cellular proliferation; and assessment of necrosis. The WHO and the European Neuroendocrine Tumor Society (ENETS) both incorporate mitotic count and Ki-67 proliferation for the classification of gastroenteropancreatic NETs (GEP-NETs).

Tumors fall into one of the following three grades:
G1: Well differentiated histology, low grade
G2: Well differentiated histology, intermediate grade
G3: Poorly differentiated histology, high grade

Staging of NETs is adapted from the American Joint Committee on Cancer staging of more common cancers with the same site of origin, but other staging systems are also in use.

Neuroendocrine tumors can be functional or nonfunctional, with each having a distinct clinical profile. Functional NETs are characterized by symptoms caused by the biologically active compounds they secrete. Neuroendocrine tumors produce peptides and hormones that are responsible for the characteristic symptoms. Known products of well-differentiated NETs include: amines (e.g., serotonin, 5-hydroxytryptophan (5-HTP), norepinephrine, dopamine, histamine), polypeptides (e.g., kallikrein, pancreatic polypeptide, bradykinin, motilin, somatostatin, vasoactive intestinal peptide, neuropeptide K, substance P, neurokinin A and B), and prostaglandins (e.g., prostaglandins E and F).

Functional, non-pancreatic NETs release excess serotonin and other hormonally active products (including histamine, tachykinins, kallikrein, and prostaglandins) resulting in the symptoms collectively referred to as carcinoid syndrome (Vinik A, Hughes M, Feliberti E, et al. Carcinoid Tumors. Endotext [Internet]. Copyright @ MDText.com, Inc; 2000-2021. 2018). These symptoms most commonly include cutaneous flushing (seen in 85%) and recurrent watery diarrhea and cramping (seen in 75%-85%). Carcinoid syndrome is confirmed biochemically with documentation of elevations of serum serotonin or the serotonin metabolite 5 hydroxyindolacetic acid (5-HIAA).

Nonfunctioning NETs are not associated with abnormal hormonal secretion, can be difficult to detect, and often present at an advanced stage with secondary nonspecific symptoms. Up to 50% to 75% of patients with nonfunctioning small-bowel NETs present with metastatic disease at diagnosis.

Most patients with CS have intestinal NETs with liver metastases, which makes surgical cure very difficult in most patients.

Patients with CS diarrhea experience watery, loose stools multiple times a day, sometimes associated with considerable urgency, and the management can be very challenging. In addition to CS, patients with NETs can have other causes of diarrhea. Depending on location, NETs may cause symptoms similar to those of common conditions, such as irritable bowel syndrome, Crohns disease, peptic ulcer disease, gastritis, other digestive disorders, asthma, or pneumonia. For these reasons, NETs are often initially diagnosed at an advanced stage and are commonly misdiagnosed. Diagnosis is based on histopathology, imaging, and circulating biomarkers.

Carcinoid syndrome biomarkers include plasma 5-HIAA and pancreastatin; serum chromogranin A and serotonin. Pancreastatin is a disease-state biomarker for neuroendocrine tumors and can be used for exploratory assessment of the effect of paltusotine in Carcinoid Syndrome subjects, alone or in combination with plasma 5 HIAA, chromogranin A and serotonin levels.

Because the symptoms of CS are often nonspecific, a delay in diagnosis is common. Detected early, NETs can often be cured with surgery; unfortunately, many will eventually recur, often more than 5 to 10 years after surgery. Most NETs are diagnosed as advanced metastatic disease, however, at which point surgical cure is not possible and treatment is focused on symptom management.

Increased frequency of bowel movements, diarrhea, fecal incontinence, and cutaneous flushing correlate with decreased quality of life (QoL). When compared to non-NET cancer populations and population norms, patients with NETS have high rates of depression and cognitive impairment, decreases in overall physical function, impairment in sleep, fatigue, and anxiety.

Diarrhea has a particularly prominent adverse impact on physical, emotional, and social well-being. Interventions to improve individual daily activities and productivity can improve health-related QoL and reduce stress in patients with NETs.

Carcinoid syndrome diarrhea is largely a consequence of tumoral secretion of serotonin. In a healthy individual, serotonin is primarily found in the GI tract with most secreted by enterochromaffin cells, the cellular source of NETs. Circulating serotonin is exclusively derived from the GI tract and is an important component of normal gut function. Excess serotonin increases peristalsis, which results in reduced absorption of water and electrolytes, leading to diarrhea. Patients with CS present with a significant increase in serotonin plasma levels and, consequently, in the levels of soluble urinary metabolite 5-HIAA. Reduction in serotonin production, reflected in lower levels of 5-HIAA, is associated with improvement in CS diarrhea.

Diarrhea associated with CS tends to be watery after the first 1 or 2 movements, and the frequency of bowel movements can range from 2 to 5 in a day to more than 20, which can be extremely debilitating and deleterious to QoL. The presence of watery diarrhea, in association with cutaneous facial flushing (especially when associated with evidence of elevated serotonin blood levels), and radiographic evidence of malignancy is almost always diagnostic of CS diarrhea.

Diarrhea is described as an increase in the frequency of bowel movements or a decrease in the consistency of stools that causes the discharge of watery, loose stools. The severity of diarrhea is determined by the size and number of stools passed within a period of time.

Severe diarrhea means having more than 10 loose, watery stools in a single day (24 hours). Moderate diarrhea means having more than a few but not more than 10 diarrhea stools in a day. Mild diarrhea means having a few diarrhea stools in a day.

In some embodiments, clinical symptom improvements are measured as change from baseline in stool consistency per Bristol Stool Scale. The Bristol Stool Scale is a medical aid designed to classify feces on a scale from 1 to 7 according to increasing wateriness.

In some embodiments, Compound A, or a pharmaceutically acceptable salt thereof (e.g., Compound A-HCl), is used to improve quality of life (QoL) in a human with CS. In some embodiments, the improvement in QoL comprises: managing the symptoms of hormone excess associated with CS. In some embodiments, the improvement in QoL comprises: decreasing the frequency of bowel movements, decreasing the frequency of diarrhea, decreasing the frequency of fecal incontinence, decreasing the frequency of cutaneous flushing, or a combination thereof.

Nearly 90% of the well-differentiated NETs that cause CS express somatostatin receptors (SSTRs) and 80% of patients with carcinoid syndrome respond to octreotide. The dominant target for octreotide is somatostatin type 2 receptor (SST2). Therefore, somatostatin receptor imaging can be useful for localizing NETs not visualized with CT or MRI, providing whole body images of tumor burden as well as documenting tumoral expression of somatostatin receptors prior to treatment with SRLs. Currently, positron emission tomography (PET) tracers for SSTR imaging (eg, ⁶⁸Ga-Dotatate and ⁶⁸Ga-Dotatoc, and ⁶⁴Cu-Dotatate) in combination with CT are preferred relative to ¹¹¹In-pentetreotide imaging because of increased spatial resolution and sensitivity for detection of small tumors.

While long-acting somatostatin receptor ligand (SRL) therapies are mainstay treatments for carcinoid syndrome, relief of carcinoid syndrome symptoms at labeled doses is inadequate for many patients. Long acting SRL therapies have also demonstrated efficacy in prolonging progression free survival in patients with non functional, metastatic, well or moderately differentiated enteropancreatic NETs. Long-acting SRL therapies may be associated with significant dose to dose exposure variability related to injection techniques. For example, nurses at MD Anderson Cancer Center successfully delivered only 52% of 328 octreotide long acting repeatable (LAR) Depot injections to the intramuscular space when evaluated by computed tomography (Boyd A E, DeFord L L, Mares J E, et al. Improving the success rate of gluteal intramuscular injections. Pancreas. 2013; 42 (5): 878-882). Negative experiences with chronic injections of long acting SRL therapies, particularly octreotide LAR, also have been identified in patients with carcinoid syndrome, including injection site pain or soreness (Adams J R, Ray D, Willmon R, Pulgar S, Dasari A. Living With Neuroendocrine Tumors: Assessment of Quality of Life Through a Mobile Application. JCO Clin Cancer Inform. 2019; 3:1-10).

Ongoing diarrhea after initiation of therapy with selective somatostatin analogues (SSAs) is common and most commonly due to incompletely controlled CS. In such circumstances, the diarrhea retains the characteristics of the diarrhea prior to initiation of therapy but occurs less frequently.

Use of SSAs, approved by the US Food and Drug Administration (FDA) for the management and control of CS, may contribute to worsening of diarrhea. One of the more common adverse reactions of chronic use of SSAs is steatorrhea, with frequency of 26% to 65% for lanreotide (dose related), and 36% to 61% for octreotide. Steatorrhea associated with SSAs results from inhibition of meal-stimulated digestive enzymes from the pancreas.

Despite use of SSAs, patients may become refractory to treatment and experience symptom progression. Treatment options used in clinical practice for refractory CS include SSA dose escalation, interferon, and surgical, embolic, and radiation therapies. Despite these treatment strategies, patients are likely to continue to experience progression of CS diarrhea symptoms and continue to have 4 or more daily bowel movements. In some embodiments, Compound A, or a pharmaceutically acceptable salt thereof, is used in the treatment of CS in a human with CS, wherein the human with CS is refractory to treatment with somatostatin analogues (SSAs).

A daily oral treatment such as Compound A may achieve higher drug concentrations in the liver, which is the most common source of the vasoactive mediators causing carcinoid syndrome symptoms. Compound A, an orally administered nonpeptide somatostatin type 2 receptor (SST2) agonist, has the potential to improve treatment outcomes, achieving symptom control while eliminating painful injections.

Compound A is a somatostatin modulator that is useful in the methods of treatment described herein.

### Compound A

As used herein, Compound A refers to 3-(4-(4-amino-piperidin-1-yl)-3-(3,5-difluoro-phenyl)-quinolin-6-yl)-2-hydroxy-benzonitrile, which has the chemical structure shown below.

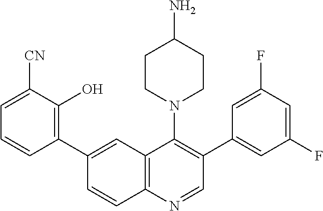 [SMILES: N#Cc1cccc(-c2ccc3ncc(-c4cc(F)cc(F)c4)c(N4CCC(N)CC4)c3c2)c1O]

Compound A is also known as “paltusotine”. Other names may be known or given to Compound A.

Compound A is a selective nonpeptide SST2 biased agonist. In clinical studies, Compound A was shown to have an estimated bioavailability of about 70% and an observed half-life of about 42 to about 50 hours. In some embodiments, Compound A is used to treat acromegaly, neuroendocrine tumors, or both. In some embodiments, Compound A is used to treat acromegaly. In some embodiments, Compound A is used to treat neuroendocrine tumors.

In some embodiments, the free base form of Compound A is incorporated into the formulations described herein. In some embodiments, Compound A is incorporated into the formulations described herein as a pharmaceutically acceptable salt. In some embodiments, Compound A is incorporated into the formulations described herein as a pharmaceutically acceptable solvate.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use,

Weinheim/Zürich: Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound disclosed herein with an acid. In some embodiments, the compound disclosed herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, Compound A is incorporated into the formulations described herein as a pharmaceutically acceptable salt form that is selected from Compound A hydrochloride and Compound A methanesulfonic acid. In some embodiments, the Compound A salt form is Compound A monohydrochloride. In some embodiments, the Compound A salt form is Compound A dihydrochloride. In some embodiments, the Compound A salt form is Compound A monomethanesulfonic acid. In some embodiments, the Compound A salt form is Compound A dimethanesulfonic acid.

In one aspect, Compound A monohydrochloride (Compound A-HCl) is incorporated into the pharmaceutical compositions described herein. Compound A monohydrochloride (Compound A-HCl), also known as 3-[4-(4-amino-piperidin-1-yl)-3-(3,5-difluoro-phenyl)-quinolin-6-yl]-2-hydroxy-benzonitrile monohydrochloride and paltusotine monohydrochloride, has the following structure:

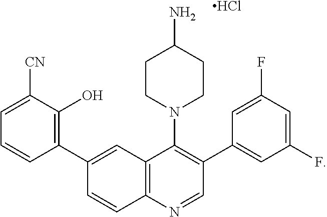 [SMILES: Cl ; N#Cc1cccc(-c2ccc3ncc(-c4cc(F)cc(F)c4)c(N4CCC(N)CC4)c3c2)c1O]

In some embodiments, the Compound A salt form is amorphous. In some embodiments, Compound A monohydrochloride is amorphous

In some embodiments, the Compound A salt form is crystalline. In some embodiments, Compound A monohydrochloride is crystalline.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

Therapeutic agents that are administrable to mammals, such as humans, must be prepared by following regulatory guidelines. Such government regulated guidelines are referred to as Good Manufacturing Practice (GMP). GMP guidelines outline acceptable contamination levels of active therapeutic agents, such as, for example, the amount of residual solvent in the final product. Preferred solvents are those that are suitable for use in GMP facilities and consistent with industrial safety concerns. Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005).

Solvents are categorized into three classes. Class 1 solvents are toxic and are to be avoided. Class 2 solvents are solvents to be limited in use during the manufacture of the therapeutic agent. Class 3 solvents are solvents with low toxic potential and of lower risk to human health. Data for Class 3 solvents indicate that they are less toxic in acute or short-term studies and negative in genotoxicity studies.

Class 1 solvents, which are to be avoided, include: benzene; carbon tetrachloride; 1,2-dichloroethane; 1,1-dichloroethene; and 1,1,1-trichloroethane.

Examples of Class 2 solvents are: acetonitrile, chlorobenzene, chloroform, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, N-methylpyrrolidine, nitromethane, pyridine, sulfolane, tetralin, toluene, 1,1,2-trichloroethene and xylene.

Class 3 solvents, which possess low toxicity, include: acetic acid, acetone, anisole, 1-butanol, 2-butanol, butyl acetate, tert-butylmethyl ether (MTBE), cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran.

Residual solvents in active pharmaceutical ingredients (APIs) originate from the manufacture of API. In most cases, the solvents are not completely removed by practical manufacturing techniques. Appropriate selection of the solvent for the synthesis of APIs may enhance the yield, or determine characteristics such as crystal form, purity, and solubility. Therefore, the solvent is a critical parameter in the synthetic process.

In some embodiments, compositions comprising Compound A-HCl include a residual amount of an organic solvent(s). In some embodiments, compositions comprising Compound A-HCl comprise a residual amount of a Class 2 or Class 3 solvent. In some embodiments, compositions comprising Compound A-HCl comprise a residual amount of a solvent selected from ethyl acetate, isopropyl acetate, tert-butylmethylether, heptane, isopropanol, methanol, acetone, dimethylformamide, tetrahydrofuran, 1,4-dioxane, 2-methyltetrahydrofuran, toluene, and ethanol.

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes of administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound disclosed herein, or a pharmaceutically acceptable salt thereof, and a co-agent, are both administered to a patient simultaneously in the form of a single entity or dosage. The term “non-fixed combination” means that the active ingredients, e.g. a compound disclosed herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “article of manufacture” and “kit” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though are not limited to delivery via enteral routes (including oral) administration, although the most suitable route may depend upon for example the condition and disorder of the recipient.

In some embodiments, pharmaceutical compositions suitable for oral administration are presented as discrete units such as capsules or tablets each containing a predetermined amount of the active ingredient; or as a powder or granules.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets for identification or to characterize different combinations of active compound doses.

Conventional techniques to manufacture solid oral dosage forms include, but are not limited to, one or a combination of methods: (1) dry mixing, (2) direct compression, (3) milling, (4) dry or non-aqueous granulation, or (5) wet granulation. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

It should be understood that in addition to the ingredients particularly mentioned above, the compounds and compositions described herein may include other agents conventional in the art having regard to the type of formulation in question, for example those suitable for oral administration may include flavoring agents.

Provided herein are tablets comprising Compound A, or a pharmaceutically acceptable salt thereof. In some embodiments, the tablet comprises: Compound A-HCl, or solvate thereof, dispersed in a polymer matrix formed from a pharmaceutically acceptable polymer; one or more pharmaceutical acceptable ingredients selected from the group consisting of one or more diluents, one or more disintegrants, one or more lubricants, one or more glidants; and optionally one or more film coating agents.

In some embodiments, described herein is a spray-dried solid dispersion comprising (a) Compound A-HCl, or solvate thereof; and (b) a pharmaceutically acceptable polymer; wherein Compound A-HCl, or solvate thereof, is dispersed in a polymer matrix formed from the pharmaceutically acceptable polymer.

In some embodiments, described herein are tablets prepared with the spray-dried solid dispersions described herein.

### Spray-Dried Solid Dispersion (SDD)

The amorphous state for most small molecule drugs is thermodynamically unstable and, unless the glass transition temperature (Tg) is sufficiently high, also kinetically unstable. However, the amorphous state can be stabilized by dilution of the drug in an excipient matrix. When an amorphous molecule is dispersed in a high Tg matrix, low molecular mobility provides a diffusion barrier which inhibits molecular mobility that is required for phase separation upon storage. Phase separation into drug rich domains is the precursor to forming crystal nuclei and eventually widespread crystallization which results in a lost solubility advantage. In some embodiments, pharmaceutically acceptable polymers for use in preparing spray-dried solid dispersions are polymers with a high Tg. In cases when the active pharmaceutical ingredient (API) and excipient are not thermodynamically miscible with one another in the solid state, the spray-dried dispersion (SDD) is formulated such that the resulting Tg of the mixture, including absorbed water, is at least 10° C. to 20° C. greater than typical storage conditions. Further, considerations must be made with respect to water uptake during storage, either through selection of a non-hygroscopic polymer or packaging configuration, as adsorbed water will plasticize the dispersion and lower the Tg.

In some embodiments, Compound A-HCl, or solvate thereof, in the spray-dried solid dispersions described herein is substantially amorphous.

In some embodiments, the pharmaceutically acceptable polymer comprises polymers of: cellulose optionally functionalized with any combination of alkyl ethers, alkyl esters, phthalate esters; vinyl alcohol; vinyl acetate; propylene glycol; pyrrolidone; vinylpyrrolidone, oxyethylene; oxypropylene; methacrylic acid; methyl methacrylate; ethylene glycol; ethylene glycol glycerides; ethylene oxide; propylene oxide; 2-ethyl-2-oxazoline; maleic acid; methyl vinyl ether; vinyl caprolactam; or combinations thereof.

In some embodiments, the pharmaceutically acceptable polymer is hydroxypropyl methylcellulose (HPMC), hydroxypropyl methyl cellulose acetate succinate (HPMCAS), hydroxypropyl cellulose (HPC), methyl cellulose, hydroxyethyl methyl cellulose, hydroxyethyl cellulose acetate, hydroxyethyl ethyl cellulose, polyvinyl alcohol polyvinyl acetate copolymers, polyethylene glycol, polyethylene glycol polypropylene glycol copolymers, polyvinylpyrrolidone (PVP), polyvinylpyrrolidone polyvinyl acetate copolymers (PVP/VA), polyethylene polyvinyl alcohol copolymers, polyoxyethylene-polyoxypropylene block copolymers, cellulose acetate phthalate (CAP), hydroxypropyl methyl-cellulose phthalate (HPMCP), co-polymer of methacrylic acid and methyl methacrylate, polyethylene glycol glycerides composed of mono-, di- and triglycerides and mono- and diesters of polyethylene glycol, hydroxypropylcellulose, copolymers of ethylene oxide and propylene oxide blocks, poly(2-ethyl-2-oxazoline), poly(maleic acid/methyl vinyl ether), polyvinyl caprolactam-polyvinyl acetate-polyethylene glycol graft copolymer, ethylene oxide/propylene oxide tetra functional block copolymer, d-alpha tocopheryl polyethylene glycol 1000 succinate, or combinations thereof.

In some embodiments, the spray-dried dispersion further comprises a dispersion polymer. Dispersion polymers are selected from hydroxypropyl methylcellulose (HPMC), hypromellose acetate succinate (hydroxypropyl methyl cellulose acetate succinate; HPMCAS, such as HPMCAS-H, HPMCAS-L, or HPMCAS-M), hydroxypropyl cellulose (HPC), methyl cellulose, hydroxyethyl methyl cellulose, hydroxyethyl cellulose acetate, hydroxyethyl ethyl cellulose, polyvinyl alcohol polyvinyl acetate copolymers, polyethylene glycol, polyethylene glycol polypropylene glycol copolymers, polyvinylpyrrolidone (PVP), polyvinylpyrrolidone polyvinyl acetate copolymers (PVP/VA), polyethylene polyvinyl alcohol copolymers, polyoxyethylene-polyoxypropylene block copolymers, and combinations thereof.

HPMCAS is a cellulosic polymer with four types of substituents semirandomly substituted on the hydroxyls: methoxy, hydroxypropyloxy, acetate, and succinate. The polymer is available in three grades: L, M and H, based on the content of acetyl and succinoyl groups (wt %) in the HPMCAS molecule. Grade L: 5-9% by weight acetate, 14-18% by weight succinate, 20-24% by weight methoxy, 5-9% by weight hydroxypropyloxy. Grade M: 7-11% by weight acetate, 10-14% by weight succinate, 21-25% by weight methoxy, 5-9% by weight hydroxypropyloxy. Grade H: 10-14% by weight acetate, 4-8% by weight succinate, 22-26% by weight methoxy, 6-10% by weight hydroxypropyloxy.

In some embodiments, the pharmaceutically acceptable polymer is selected from PVP/VA 64, PVP 30, HPMCAS-L, HPMCAS-M, HPMCAS-H, Eudragit L100-55, poly(methacrylic acid-co-methyl methacrylate) (PMMAMA, or trade name Eudragit L100), Eudragit EPO, HPMC E15, HPMC E3, HPMC E5, HPMCP-HP55, and Soluplus.

In some embodiments, the pharmaceutically acceptable polymer is selected from PVP/VA 64 and HPMCAS-M. In some embodiments, the pharmaceutically acceptable polymer is PVP/VA 64. In some embodiments, the pharmaceutically acceptable polymer is HPMCAS-M.

In some embodiments, the weight ratio of Compound A-HCl, or solvate thereof, to the dispersion polymer is between about 1:10 and about 10:1. In some embodiments, the weight ratio of Compound A-HCl, or solvate thereof, to the dispersion polymer is between about 1:1 and about 1:10. In some embodiments, the weight ratio of Compound A-HCl, or solvate thereof, to the dispersion polymer is between about 1:3 and about 1:8. In some embodiments, the weight ratio of Compound A-HCl, or solvate thereof, to the dispersion polymer is between about 1:4 and about 1:7. In some embodiments, the weight ratio of Compound A-HCl, or solvate thereof, to the dispersion polymer is between about 1:4 and about 1:6. In some embodiments, the weight ratio of Compound A-HCl, or solvate thereof, to the dispersion polymer is between about 1:5 and about 1:6. In some embodiments, the weight ratio of Compound A-HCl, or solvate thereof, to the dispersion polymer is about 1:10. In some embodiments, the weight ratio of Compound A-HCl, or solvate thereof, to the dispersion polymer is about 1:9. In some embodiments, the weight ratio of Compound A-HCl, or solvate thereof, to the dispersion polymer is about 1:8. In some embodiments, the weight ratio of Compound A-HCl, or solvate thereof, to the dispersion polymer is about 1:7. In some embodiments, the weight ratio of Compound A-HCl, or solvate thereof, to the dispersion polymer is about 1:6. In some embodiments, the weight ratio of Compound A-HCl, or solvate thereof, to the dispersion polymer is about 1:5. In some embodiments, the weight ratio of Compound A-HCl, or solvate thereof, to the dispersion polymer is about 1:4. In some embodiments, the weight ratio of Compound A-HCl, or solvate thereof, to the dispersion polymer is about 1:3. In some embodiments, the weight ratio of Compound A-HCl, or solvate thereof, to the dispersion polymer is about 1:2. In some embodiments, the weight ratio of Compound A-HCl, or solvate thereof, to the dispersion polymer is about 1:1.

In some embodiments, the spray-dried solid dispersion comprises at least 5% by weight of Compound A-HCl, or solvate thereof. In some embodiments, the spray-dried solid dispersion comprises at least 10% by weight of Compound A-HCl, or solvate thereof. In some embodiments, the spray-dried solid dispersion comprises at least 15% by weight of Compound A-HCl, or solvate thereof. In some embodiments, the spray-dried solid dispersion comprises at least 20% by weight of Compound A-HCl, or solvate thereof. In some embodiments, the spray-dried solid dispersion comprises at least 25% by weight of Compound A-HCl, or solvate thereof. The % amounts are calculated based on the free base, i.e. Compound A.

In some embodiments, the spray-dried solid dispersion comprises about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% by weight of Compound A-HCl, or solvate thereof. In some embodiments, the spray-dried solid dispersion comprises about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or about 35% by weight of Compound A-HCl, or solvate thereof. In some embodiments, the spray-dried solid dispersion comprises about 15% of Compound A-HCl, or solvate thereof. In some embodiments, the spray-dried solid dispersion comprises about 35% of Compound A-HCl, or solvate thereof. The % amounts are calculated based on the free base, i.e. Compound A.

In some embodiments, the spray-dried solid dispersion comprises about 5% by weight of Compound A-HCl, or solvate thereof. In some embodiments, the spray-dried solid dispersion comprises about 6% by weight of Compound A-HCl, or solvate thereof. In some embodiments, the spray-dried solid dispersion comprises about 7% by weight of Compound A-HCl, or solvate thereof. In some embodiments, the spray-dried solid dispersion comprises about 8% by weight of Compound A-HCl, or solvate thereof. In some embodiments, the spray-dried solid dispersion comprises about 9% by weight of Compound A-HCl, or solvate thereof. In some embodiments, the spray-dried solid dispersion comprises about 10% by weight of Compound A-HCl, or solvate thereof. In some embodiments, the spray-dried solid dispersion comprises about 11% by weight of Compound A-HCl, or solvate thereof. In some embodiments, the spray-dried solid dispersion comprises about 12% by weight of Compound A-HCl, or solvate thereof. In some embodiments, the spray-dried solid dispersion comprises about 13% by weight of Compound A-HCl, or solvate thereof. In some embodiments, the spray-dried solid dispersion comprises about 14% by weight of Compound A-HCl, or solvate thereof. In some embodiments, the spray-dried solid dispersion comprises about 15% by weight of Compound A-HCl, or solvate thereof. In some embodiments, the spray-dried solid dispersion comprises about 16% by weight of Compound A-HCl, or solvate thereof. In some embodiments, the spray-dried solid dispersion comprises about 17% by weight of Compound A-HCl, or solvate thereof. In some embodiments, the spray-dried solid dispersion comprises about 18% by weight of Compound A-HCl, or solvate thereof. In some embodiments, the spray-dried solid dispersion comprises about 19% by weight of Compound A-HCl, or solvate thereof. In some embodiments, the spray-dried solid dispersion comprises about 20% by weight of Compound A-HCl, or solvate thereof. In some embodiments, the spray-dried solid dispersion comprises about 21% by weight of Compound A-HCl, or solvate thereof. In some embodiments, the spray-dried solid dispersion comprises about 22% by weight of Compound A-HCl, or solvate thereof. In some embodiments, the spray-dried solid dispersion comprises about 23% by weight of Compound A-HCl, or solvate thereof. In some embodiments, the spray-dried solid dispersion comprises about 24% by weight of Compound A-HCl, or solvate thereof. In some embodiments, the spray-dried solid dispersion comprises about 25% by weight of Compound A-HCl, or solvate thereof. In some embodiments, the spray-dried solid dispersion comprises about 26% by weight of Compound A-HCl, or solvate thereof. In some embodiments, the spray-dried solid dispersion comprises about 27% by weight of Compound A-HCl, or solvate thereof. In some embodiments, the spray-dried solid dispersion comprises about 28% by weight of Compound A-HCl, or solvate thereof. In some embodiments, the spray-dried solid dispersion comprises about 29% by weight of Compound A-HCl, or solvate thereof. In some embodiments, the spray-dried solid dispersion comprises about 30% by weight of Compound A-HCl, or solvate thereof. In some embodiments, the spray-dried solid dispersion comprises about 31% by weight of Compound A-HCl, or solvate thereof. In some embodiments, the spray-dried solid dispersion comprises about 32% by weight of Compound A-HCl, or solvate thereof. In some embodiments, the spray-dried solid dispersion comprises about 33% by weight of Compound A-HCl, or solvate thereof. In some embodiments, the spray-dried solid dispersion comprises about 34% by weight of Compound A-HCl, or solvate thereof. In some embodiments, the spray-dried solid dispersion comprises about 35% by weight of Compound A-HCl, or solvate thereof. The % amounts are calculated based on the free base, i.e. Compound A.

In some embodiments, the spray-dried solid dispersion further comprises a non-aqueous solvent. In some embodiments, the non-aqueous solvent is present in detectable amounts. In some embodiments, the spray-dried solid dispersion is non-aqueous solvent free.

In some embodiments, the spray-dried solid dispersion further comprises a non-aqueous solvent selected from the group consisting of tert-butanol, n-propanol, n-butanol, isopropanol, ethanol, methanol, acetone, ethyl acetate, acetonitrile, methyl ethyl ketone, methyl isobutyl ketone, methyl acetate, and mixtures thereof. In some embodiments, the spray-dried solid dispersion further comprises a non-aqueous solvent selected from the group consisting of methanol, acetone, and mixtures thereof. In some embodiments, the spray-dried solid dispersion further comprises methanol.

### Tablets

In one aspect, described herein is a tablet comprising: Compound A-HCl, or solvate thereof, dispersed in a polymer matrix formed from a pharmaceutically acceptable polymer; one or more pharmaceutical acceptable ingredients selected from the group consisting of one or more diluents, one or more disintegrants, one or more lubricants, one or more glidants; and optionally one or more film coating agents.

In some embodiments, Compound A-HCl, or solvate thereof, dispersed in a polymer matrix formed from a pharmaceutically acceptable polymer is the spray-dried solid dispersion described herein.

In some embodiments, tablets comprise about 2% by weight to about 20% by weight of Compound A-HCl, or solvate thereof. In some embodiments, tablets comprise about 2% by weight to about 15% by weight of Compound A-HCl, or solvate thereof.

In some embodiments, tablets comprise about 10% by weight to about 30% by weight of the polymer matrix formed from the pharmaceutically acceptable polymer. In some embodiments, tablets comprise about 20% by weight to about 35% by weight of the polymer matrix formed from the pharmaceutically acceptable polymer.

In some embodiments, tablets comprise about 2% by weight to about 10% by weight of Compound A-HCl, or solvate thereof, dispersed in about 10% by weight to about 30% by weight of a polymer matrix formed from a pharmaceutically acceptable polymer.

In some embodiments, tablets comprise about 2% by weight to about 10% by weight of Compound A-HCl, or solvate thereof, dispersed in about 10% by weight to about 30% by weight of a polymer matrix formed from a pharmaceutically acceptable polymer; about 40% by weight to about 80% by weight of one or more pharmaceutical acceptable ingredients selected from the group consisting of one or more diluents, one or more disintegrants, one or more lubricants, one or more glidants; and optionally less than about 5% by weight of one or more film coating agents.

In some embodiments, in addition to the spray-dried solid dispersion, additional excipients in the tablets comprise one or more diluents, one or more disintegrants, one or more lubricants, one or more glidants, or any combination thereof. In some embodiments, in addition to the spray-dried solid dispersion, additional excipients in the tablets comprise microcrystalline cellulose, mannitol, crospovidone, colloidal silicon dioxide, and magnesium stearate.

In some embodiments, the tablet comprises one or more fillers/binders/diluents. Fillers/binders/diluents are selected from celluloses (such as microcrystalline cellulose, carboxymethylcellulose, ethyl cellulose and methyl cellulose), starch, gelatin, sugars (such as sucrose, glucose, dextrose, mannitol, and lactose), natural and synthetic gums (such as acacia, sodium alginate, panwar gum, and ghatti gum), polyvinylpyrrolidinone, polyethylene glycol, waxes, and any combinations thereof. In some embodiments, tablets comprise microcrystalline cellulose, and mannitol.

In some embodiments, the one or more fillers/binders/diluents in the tablets described herein comprise between about 20% and about 80% by weight of the total tablet weight. In some embodiments, the one or more fillers/binders/diluents in the tablets described herein comprise between about 40% and about 65% by weight of the total tablet weight. In some embodiments, the one or more fillers/binders/diluents in the tablets described herein comprise between about 50% and about 65% by weight of the total tablet weight. In some embodiments, the one or more fillers/binders/diluents in the tablets described herein comprise about 45%, about 50%, about 55%, about 60%, about 65%, or about 70% by weight of the total tablet weight. In some embodiments, the one or more fillers/binders/diluents in the tablets described herein comprise about 58% by weight of the total tablet weight. In some embodiments, less than 70% by weight, less than 65% by weight, less than 60% by weight, less than 55% by weight, or less than 50% by weight of the total tablet weight comprise one or more fillers/binders/diluents. In some embodiments, less than 60% by weight of the total tablet weight comprise one or more fillers/binders/diluents.

In some embodiments, tablets comprise one or more disintegrants. Disintegrants are selected from croscarmellose sodium, crospovidone, sodium starch glycolate, veegum HV, methylcellulose, agar, bentonite, cellulose, carboxymethyl cellulose, and any combination thereof. In some embodiments, tablets comprise crospovidone.

In some embodiments, the one or more disintegrants in the tablets described herein comprise between about 2% and about 30% by weight of the total tablet weight. In some embodiments, the one or more disintegrants in the tablets described herein comprise between about 5% and about 20% by weight of the total tablet weight. In some embodiments, the one or more disintegrants in the tablets described herein comprise between about 10% and about 20% by weight of the total tablet weight. In some embodiments, the one or more disintegrants in the tablets described herein comprise about 5%, about 6%, about 7%, about 8%, about 9%, about 10%, about 11%, about 12%, about 13%, about 14%, about 15%, about 16%, about 17%, about 18%, about 19%, or about 20% by weight of the total tablet weight. In some embodiments, the one or more disintegrants in the tablets described herein comprise about 15% by weight of the total tablet weight. In some embodiments, less than 20% by weight of the total tablet weight comprise one or more disintegrants.

In some embodiments, tablets comprise one or more lubricants. Lubricants are selected from talc, magnesium stearate, calcium stearate, stearic acid, sodium stearyl fumarate, glyceryl behenate, hydrogenated vegetable oils, polyethylene glycol, and any combinations thereof. In some embodiments, tablets comprise magnesium stearate.

In some embodiments, the one or more lubricants in the tablets described herein comprise between about 0.1% and about 5% by weight of the total tablet weight. In some embodiments, the one or more lubricants in the tablets described herein comprise between about 0.1% and about 2% by weight of the total tablet weight. In some embodiments, the one or more lubricants in the tablets described herein comprise between about 0.1% and about 1% by weight of the total tablet weight. In some embodiments, the one or more lubricants in the tablets described herein comprise about 0.1%, about 0.2%, about 0.3%, about 0.4%, about 0.5%, about 0.6%, about 0.7%, about 0.8%, about 0.9%, or about 1% by weight of the total tablet weight. In some embodiments, the one or more lubricants in the tablets described herein comprise about 0.5% by weight of the total tablet weight. In some embodiments, less than 2% by weight of the total tablet weight comprise one or more lubricants. In some embodiments, less than 1% by weight of the total tablet weight comprise one or more lubricants.

In some embodiments, tablets comprise one or more glidants. A glidant is a substance that is added to a powder to improve its flowability. Examples of glidants include magnesium stearate, colloidal silicon dioxide, starch and talc. In some embodiments, tablets comprise colloidal silicon dioxide.

In some embodiments, the one or more lubricants in the tablets described herein comprise between about 0.1% and about 5% by weight of the total tablet weight. In some embodiments, the one or more lubricants in the tablets described herein comprise between about 0.1% and about 2% by weight of the total tablet weight. In some embodiments, the one or more lubricants in the tablets described herein comprise between about 0.5% and about 1.5% by weight of the total tablet weight. In some embodiments, the one or more lubricants in the tablets described herein comprise about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2% by weight of the total tablet weight. In some embodiments, the one or more lubricants in the tablets described herein comprise about 1% by weight of the total tablet weight. In some embodiments, less than 2% by weight of the total tablet weight comprise one or more lubricants. In some embodiments, less than 1.5% by weight of the total tablet weight comprise one or more lubricants.

### Additional Excipients

In some embodiments, the tablet described herein comprises additional excipients including, but not limited, to buffering agents, glidants, preservatives, and coloring agents. Additional excipients such as bulking agents, tonicity agents, and chelating agents are within the scope of the embodiments.

Non-limiting examples of buffering agents include, but are not limited to, sodium bicarbonate, potassium bicarbonate, magnesium hydroxide, magnesium lactate, magnesium glucomate, aluminum hydroxide, aluminum hydroxide/sodium bicarbonate co precipitate, a mixture of an amino acid and a buffer, a mixture of aluminum glycinate and a buffer, a mixture of an acid salt of an amino acid and a buffer, and a mixture of an alkali salt of an amino acid and a buffer. Additional buffering agents include sodium citrate, sodium tartarate, sodium acetate, sodium carbonate, sodium polyphosphate, potassium polyphosphate, sodium pyrophosphate, potassium pyrophosphate, disodium hydrogenphosphate, dipotassium hydrogenphosphate, trisodium phosphate, tripotassium phosphate, sodium acetate, potassium metaphosphate, magnesium oxide, magnesium hydroxide, magnesium carbonate, magnesium silicate, calcium acetate, calcium glycerophosphate, calcium chloride, calcium hydroxide, calcium lactate, calcium carbonate, calcium bicarbonate, and other calcium salts.

In some embodiments, the tablet described herein comprises a preservative. Preservatives include anti-microbials, anti-oxidants, and agents that enhance sterility. Exemplary preservatives include ascorbic acid, ascorbyl palmitate, BHA, BHT, citric acid, erythorbic acid, fumaric acid, malic acid, propyl gallate, sodium ascorbate, sodium bisulfate, sodium metabisulfite, sodium sulfite, parabens (methyl-, ethyl-, butyl-), benzoic acid, potassium sorbate, vanillin, and the like.

In some embodiments, the tablet described herein comprises a coloring agent for identity and/or aesthetic purposes of the resultant liquid form. Suitable coloring agents illustratively include FD&C Red No. 3, FD&C Red No. 20, FD&C Red No. 40, FD&C Yellow No. 6, FD&C Blue No. 2, D&C Green No. 5, D&C Orange No. 5, caramel, ferric oxide and mixtures thereof.

Additional excipients are contemplated in the tablet embodiments. These additional excipients are selected based on function and compatibility with the tablet compositions described herein and may be found, for example in Remington: The Science and Practice of Pharmacy, Nineteenth Ed (Easton, PA: Mack Publishing Company, 1995); Hoover, John E., Remington's Pharmaceutical Sciences, (Easton, PA: Mack Publishing Co 1975); Liberman, H. A. and Lachman, L., Eds., Pharmaceutical Dosage Forms (New York, NY: Marcel Decker 1980); and Pharmaceutical Dosage Forms and Drug Delivery Systems, Seventh Ed (Lippincott Williams & Wilkins 1999), herein incorporated by reference in their entirety.

In further embodiments, the tablets described herein are coated tablets, such as enteric-coated tablets, sugar-coated, or film-coated tablets.

In one embodiment, the individual unit dosages also include film coatings, which disintegrate upon oral ingestion or upon contact with diluent. In one embodiment, these formulations are manufactured by conventional techniques.

Compressed tablets are solid dosage forms prepared by compacting the bulk blend formulations described above. In various embodiments, compressed tablets which are designed to dissolve in the mouth will include one or more flavoring agents. In other embodiments, the compressed tablets will include a film surrounding the final compressed tablet. In some embodiments, the film coating aids in patient compliance (e.g., Opadry® coatings or sugar coating). Film coatings comprising Opadry® typically range from about 1% to about 5% of the tablet weight. In other embodiments, the compressed tablets include one or more excipients.

Provided herein are film-coated tablets forms, which comprise: a combination of an active ingredient (e.g. Compound A-HCl) and one or more tableting excipients to form a tablet core and subsequently coating the core. The tablet cores are produced using conventional tableting processes and with subsequent compression and coating.

Enteric-coatings are coatings that resist the action of stomach acid but dissolve or disintegrate in the intestine.

In one aspect, the oral solid dosage form disclosed herein include an enteric coating(s). Enteric coatings include one or more of the following: cellulose acetate phthalate; methyl acrylate-methacrylic acid copolymers; cellulose acetate succinate; hydroxy propyl methyl cellulose phthalate; hydroxy propyl methyl cellulose acetate succinate (hypromellose acetate succinate); polyvinyl acetate phthalate (PVAP); methyl methacrylate-methacrylic acid copolymers; methacrylic acid copolymers, cellulose acetate (and its succinate and phthalate version); styrol maleic acid co-polymers; polymethacrylic acid/acrylic acid copolymer; hydroxyethyl ethyl cellulose phthalate; hydroxypropyl methyl cellulose acetate succinate; cellulose acetate tetrahydrophtalate; acrylic resin; shellac.

An enteric coating is a coating put on a tablet, pill, capsule, pellet, bead, granule, particle, etc. so that it doesn't dissolve until it reaches the small intestine.

Sugar-coated tablets are compressed tablets surrounded by a sugar coating, which may be beneficial in covering up objectionable tastes or odors and in protecting the tablets from oxidation.

Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets. In some embodiments, tablets are coated with water soluble, pH independent film coating which allows for immediate disintegration for fast, active release (e.g. Opadry products).

### Dosage in the Tablet

In some embodiments, the amount of Compound A-HCl, or solvate thereof, in the tablet is between about 5 mg and about 100 mg. In some embodiments, the amount of Compound A-HCl, or solvate thereof, in the tablet is between about 5 mg and about 80 mg. In some embodiments, the amount of Compound A-HCl, or solvate thereof, in the tablet is between about 5 mg and about 60 mg. In some embodiments, the amount of Compound A-HCl, or solvate thereof, in the tablet is between about 10 mg and about 40 mg.

In some embodiments, the amount of Compound A-HCl, or solvate thereof, in the tablet is about 10 mg. In some embodiments, the amount of Compound A-HCl, or solvate thereof, in the tablet is about 20 mg. In some embodiments, the amount of Compound A-HCl, or solvate thereof, in the tablet is about 30 mg. In some embodiments, the amount of Compound A-HCl, or solvate thereof, in the tablet is about 40 mg. In some embodiments, the amount of Compound A-HCl, or solvate thereof, in the tablet is about 50 mg. In some embodiments, the amount of Compound A-HCl, or solvate thereof, in the tablet is about 60 mg. In some embodiments, the amount of Compound A-HCl, or solvate thereof, in the tablet is about 70 mg. In some embodiments, the amount of Compound A-HCl, or solvate thereof, in the tablet is about 80 mg.

### Methods of Dosing and Treatment Regimens

In one embodiment, the pharmaceutical compositions disclosed herein are used as medicaments for the treatment of carcinoid syndrome in a mammal. Methods for treating carcinoid syndrome in a mammal involves administration of pharmaceutical compositions that include Compound A, or a pharmaceutically acceptable salt thereof, in therapeutically effective amounts to said mammal.

Long-acting somatostatin receptor ligands (LA-SRLs) are the first line therapy for neuroendocrine tumor (NET) syndromes including acromegaly and the carcinoid syndrome (CS). In the treatment of acromegaly, two phase 2 studies suggested that patients treated with SRLs can switch to once daily administration of Compound A (see Acrombat EDGE (NCT 03789656 and Acrobat EVOLVE (NCT 03792555)). The dose- and exposure-response data from EDGE, Evolve and in phase 1 with healthy volunteers suggest that a dose range of 40 mg to 60 mg once daily results in consistent IGF-1 suppression in patients with acromegaly.

In contrast to the efficacious doses of Compound A for acromegaly patients, CS patients may require higher doses due to malabsorption, which is associated with CS, and the potential differences in clearance and volume of distribution of Compound A between CS and acromegaly patients. In addition, the pharmacokinetics (clearance and volume of distribution) of Compound A may be different in carcinoid syndrome subjects compared to acromegalics because of differences in liver and body composition which effect metabolic activity.

The starting dose of approved long-acting SRL therapies for carcinoid syndrome is the same or higher as the starting dose for acromegaly (Sandostatin LAR Prescribing Information, 2021; Somatuline Prescribing Information, 2018). A starting dose equivalent to 40 mg of Compound A-mono-HCl is expected to be a well-tolerated starting dose for subjects with carcinoid syndrome because this dose has been well tolerated in studies on subjects with acromegaly.

In general, doses employed treatment of CS in adult humans are typically in the range of about 40 mg to about 120 mg per day of Compound A. In one embodiment, the desired dose is conveniently presented in a single dose or in divided doses administered simultaneously or at appropriate intervals, for example as two, three, four or more sub-doses per day.

In some embodiments, Compound A, or a pharmaceutically acceptable salt thereof (e.g. Compound A-monohydrochloride), is administered once a day. In some embodiments, Compound A, or a pharmaceutically acceptable salt thereof (e.g. Compound A-monohydrochloride), is administered twice a day.

In some embodiments, Compound A, or a pharmaceutically acceptable salt thereof (e.g. Compound A-monohydrochloride), is administered orally to the human on a continuous dosing schedule. In some embodiments, Compound A, or a pharmaceutically acceptable salt thereof (e.g. Compound A-monohydrochloride), is administered to the human on a continuous daily dosing schedule.

The term “continuous dosing schedule” refers to the administration of a particular therapeutic agent at regular intervals. In some embodiments, continuous dosing schedule refers to the administration of a particular therapeutic agent at regular intervals without any drug holidays from the particular therapeutic agent. In some other embodiments, continuous dosing schedule refers to the administration of a particular therapeutic agent in cycles. In some other embodiments, continuous dosing schedule refers to the administration of a particular therapeutic agent in cycles of drug administration followed by a drug holiday (for example, a wash out period or other such period of time when the drug is not administered) from the particular therapeutic agent. For example, in some embodiments the therapeutic agent is administered once a day, twice a day, three times a day, once a week, twice a week, three times a week, four times a week, five times a week, six times a week, seven times a week, every other day, every third day, every fourth day, daily for a week followed by a week of no administration of the therapeutic agent, daily for a two weeks followed by one or two weeks of no administration of the therapeutic agent, daily for three weeks followed by one, two or three weeks of no administration of the therapeutic agent, daily for four weeks followed by one, two, three or four weeks of no administration of the therapeutic agent, weekly administration of the therapeutic agent followed by a week of no administration of the therapeutic agent, or biweekly administration of the therapeutic agent followed by two weeks of no administration of the therapeutic agent. In some embodiments, daily administration is once a day. In some embodiments, daily administration is twice a day. In some embodiments, daily administration is three times a day.

The term “continuous daily dosing schedule” refers to the administration of a particular therapeutic agent every day at roughly the same time each day. In some embodiments, daily administration is once a day.

In certain embodiments wherein improvement in the status of the disease or condition in the human is not observed, the daily dose of a Compound A, or a pharmaceutically acceptable salt thereof (e.g. Compound A-monohydrochloride), is increased. In some embodiments, the frequency of administration is increased in order to provide repeat high Cₘₐₓ levels on a more regular basis (e.g. once-a-day dosing schedule is changed to a twice-a-day dosing schedule). In some embodiments, the frequency of administration is increased in order to provide maintained or more regular exposure to Compound A, or a pharmaceutically acceptable salt thereof (e.g. Compound A-monohydrochloride). In some embodiments, the frequency of administration is increased in order to provide repeat high Cₘₐₓ levels on a more regular basis and provide maintained or more regular exposure to Compound A, or a pharmaceutically acceptable salt thereof (e.g. Compound A-monohydrochloride).

Generally, a suitable dose of Compound A, or a pharmaceutically acceptable salt thereof (e.g. Compound A-monohydrochloride) for administration to a human with CS will be in the range of from about 20 mg/day to about 160 mg/day; or from about 40 mg/day to about 1200 mg/day. In other embodiments, a suitable dose of Compound A, or a pharmaceutically acceptable salt thereof (e.g. Compound A-monohydrochloride) for administration to a human will be about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, about 140 mg/day, about 150 mg/day, or about 160 mg/day. In other embodiments, a suitable dose of Compound A, or a pharmaceutically acceptable salt thereof (e.g. Compound A-monohydrochloride) for administration to a human will be about 40 mg/day. In other embodiments, a suitable dose of Compound A, or a pharmaceutically acceptable salt thereof (e.g. Compound A-monohydrochloride) for administration to a human will be about 50 mg/day. In other embodiments, a suitable dose of Compound A, or a pharmaceutically acceptable salt thereof (e.g. Compound A-monohydrochloride) for administration to a human will be about 60 mg/day. In other embodiments, a suitable dose of Compound A, or a pharmaceutically acceptable salt thereof (e.g. Compound A-monohydrochloride) for administration to a human will be about 70 mg/day. In other embodiments, a suitable dose of Compound A, or a pharmaceutically acceptable salt thereof (e.g. Compound A-monohydrochloride) for administration to a human will be about 80 mg/day. In some embodiments, dosages are administered once per day. In some embodiments, a suitable dose of Compound A, or a pharmaceutically acceptable salt thereof (e.g. Compound A-monohydrochloride) for administration to a human will be about 90 mg/day. In some embodiments, a suitable dose of Compound A, or a pharmaceutically acceptable salt thereof (e.g. Compound A-monohydrochloride) for administration to a human will be about 100 mg/day. In some embodiments, a suitable dose of Compound A, or a pharmaceutically acceptable salt thereof (e.g. Compound A-monohydrochloride) for administration to a human will be about 110 mg/day. In some embodiments, a suitable dose of Compound A, or a pharmaceutically acceptable salt thereof (e.g. Compound A-monohydrochloride) for administration to a human will be about 120 mg/day. In some embodiments, the amounts referenced above refer to the amount of Compound A-monohydrochloride.

In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the disease or condition to be treated, the mode of administration, the requirements of the individual subject, the severity of the disease or condition being treated, the identity (e.g., weight) of the human, and the particular additional therapeutic agents that are administered (if applicable), and the judgment of the practitioner.

### Treatment Based on Carcinoid Syndrome Symptoms

In clinical practice, a main treatment goal for the treatment of CS comprises managing the symptoms of hormone excess, such as frequency of bowel movements, diarrhea, fecal incontinence, and cutaneous flushing. In some embodiments, a treatment goal is a reduction in the frequency of: daily bowel movements, episodes of diarrhea, episodes of fecal incontinence, cutaneous flushing, or a combination thereof.

Somatostatin inhibits secretion of hormones and growth of NETs. Consequently, metabolically stable SSAs are the cornerstone of treatment for patients with NETs due to their inhibition of secretion of multiple hormones and reduction in the rate of disease progression. Guidelines recommend use of an SSA (octreotide or lanreotide) as the initial first-line treatment in patients with CS, with evidence that both long- and short-acting SSAs effectively control symptoms. Among patients with severe symptoms, the initial concurrent use of short-acting octreotide may be necessary until the effects of the long-acting formulation become fully effective.

In some embodiments, Compound A, or a pharmaceutically acceptable salt thereof (e.g. Compound A-monohydrochloride) is administered via a titration schedule. In some embodiments, Compound A, or a pharmaceutically acceptable salt thereof (e.g. Compound A-monohydrochloride) is administered via a titration schedule to minimize adverse events associated with the administration of Compound A, or a pharmaceutically acceptable salt thereof (e.g. Compound A-monohydrochloride). In some embodiments, titration with Compound A, or a pharmaceutically acceptable salt thereof (e.g. Compound A-monohydrochloride), enables: a subject to tolerate Compound A, or a pharmaceutically acceptable salt thereof (e.g. Compound A-monohydrochloride); to minimize adverse events associated with the administration of

Compound A, or a pharmaceutically acceptable salt thereof (e.g. Compound A-monohydrochloride); maximizes the likelihood that an optimized dose of Compound A, or a pharmaceutically acceptable salt thereof (e.g. Compound A-monohydrochloride), will be administered to the subject and tolerated; or a combination thereof. In some embodiments, titration comprises up-titration. In some embodiments, up-titration is required to manage one or more symptoms of CS.

The potential for uptitration with Compound A is based on a multi-center retrospective chart review, where the most common initial dose of octreotide LAR depot (40 mg to 60 mg every 4 weeks) was escalated subsequently up to 160 mg every 4 weeks (Strosberg J R et al., Clinical benefits of above-standard dose of octreotide LAR in patients with neuroendocrine tumors for control of carcinoid syndrome symptoms: a multicenter retrospective chart study. The Oncologist. 2014; 19:930-936).

In some embodiments, treatment with Compound A, or a pharmaceutically acceptable salt thereof (e.g. Compound A-monohydrochloride) does not require titration.

As used herein, a subject is said to “tolerate” a dose of a compound if administration of that dose to that subject does not result in an unacceptable adverse event or an unacceptable combination of adverse events. One of skill in the art will appreciate that tolerance is a subjective measure and that what may be tolerable to one patient may not be tolerable to a different patient. For example, one subject may not be able to tolerate headache, whereas a second subject may find mild headache tolerable but is not able to tolerate moderate headache, whereas a third subject is able to tolerate moderate headache but not severe headache. In some embodiments, a patient cannot tolerate treatment with an injectable somatostatin analog due to injection site rejections.

As used herein, an “adverse event” is an untoward medical occurrence that is associated with treatment with Compound A, or a pharmaceutically acceptable salt thereof. In some embodiments, an adverse event is headache, fatigue, diarrhea, pain, or combination thereof.

As used herein, an “optimized dose” refers a therapeutic dose optimized to the needs of a specific subject and is the highest dose of Compound A, or a pharmaceutically acceptable salt thereof (e.g. Compound A-monohydrochloride) that is equivalent to the needed dose of Compound A, that elicits the biological or medicinal response in the subject that is being sought and that can be tolerated by the subject, as determined by the subject, optionally in consultation with the subject's healthcare practitioner.

As used herein, “up-titration” of a compound refers to increasing the amount of a compound until the subject does not tolerate the increased amount. Up-titration can be achieved in one or more dose increments, which may be the same or different. In some embodiments, the method comprises administering Compound A, or a pharmaceutically acceptable salt thereof (e.g. Compound A-monohydrochloride), at an initial dose once daily for an initial period of time followed by up-titration to a higher dose of Compound A, or a pharmaceutically acceptable salt thereof (e.g. Compound A-monohydrochloride), once daily thereafter. In some embodiments, the initial period of time comprises one day, about one week, about two weeks, about three weeks, about four weeks, about five weeks, about six weeks, about seven weeks, about eight weeks, about nine weeks, about ten weeks, about eleven weeks, or about twelve weeks. In some embodiments, this cycle is repeated until an optimized dose is achieved.

In some embodiments, the method of titration comprises administering Compound A, or a pharmaceutically acceptable salt thereof (e.g. Compound A-monohydrochloride), at an initial dose once daily for about one week, about two weeks, about three weeks, or about four weeks, followed by up-titration to a higher dose of Compound A, or a pharmaceutically acceptable salt thereof (e.g. Compound A-monohydrochloride), once daily thereafter. In some embodiments, this cycle is repeated until an optimized dose is achieved. In some embodiments, dose adjustments are made every week, every two weeks, every three weeks or every four weeks. In some embodiments, dose adjustments are made and/or repeated to achieve consistent management of CS symptoms.

In some embodiments, the method of titration comprises administering Compound A, or a pharmaceutically acceptable salt thereof (e.g. Compound A-monohydrochloride), at an initial dose once daily for about one week, about two weeks, about three weeks, about four weeks, about five weeks, about six weeks, about seven weeks, or about eight weeks, followed by up-titration to a higher dose of Compound A, or a pharmaceutically acceptable salt thereof (e.g. Compound A-monohydrochloride), once daily thereafter. In some embodiments, this cycle is repeated until an optimized dose is achieved. In some embodiments, the method comprises administering 40 mg of Compound A-monohydrochloride once daily for a period of time, followed by up-titration to about 80 mg of Compound A-monohydrochloride once daily thereafter. In some embodiments, the method comprises administering 40 mg of Compound A-monohydrochloride once daily for about one week, about two weeks, about three weeks, about four weeks, about five weeks, about six weeks, about seven weeks, or about eight weeks, followed by up-titration to about 80 mg of Compound A-monohydrochloride once daily thereafter. In some embodiments, the method comprises administering 80 mg of Compound A-monohydrochloride once daily for a period of time, followed by up-titration to about 120 mg of Compound A-monohydrochloride once daily thereafter. In some embodiments, the method comprises administering 80 mg of Compound A-monohydrochloride once daily for about one week, about two weeks, about three weeks, about four weeks, about five weeks, about six weeks, about seven weeks, or about eight weeks, followed by up-titration to about 120 mg of Compound A-monohydrochloride once daily thereafter.

In some embodiments, the method of titration comprises the up-titration, or down-titration followed by an optional re-up-titration of Compound A, or a pharmaceutically acceptable salt thereof (e.g. Compound A-monohydrochloride).

In some embodiments, the titration schedule comprises administering Compound A, or a pharmaceutically acceptable salt thereof (e.g. Compound A-monohydrochloride), at an initial dose for about one week, two weeks, three weeks, or four weeks and, provided that the patient tolerates the initial dose, increasing the dose by an amount equal to an incremental value. In some embodiments, the incremental value for increasing the daily dose is equivalent to about 40 mg of Compound A-monohydrochloride.

In some embodiments, the initial dose is equivalent to about 40 mg or 80 mg of Compound A-monohydrochloride. In some embodiments, the initial dose is equivalent to about 40 mg of Compound A-monohydrochloride. In some embodiments, the initial dose is equivalent to about 80 mg of Compound A-monohydrochloride. In some embodiments, the daily dose of Compound A-monohydrochloride is increased by 40 mg if the CS symptoms are inadequately managed at the initial daily dose is inadequate.

In some embodiments, the titration schedule further comprises: administering

Compound A, or a pharmaceutically acceptable salt thereof, at the increased dose for about one week, two weeks, three weeks, or four weeks and provided that the patient tolerates the increased dose, further increasing the dose by an incremental value. In some embodiments, the incremental value for increasing the daily dose is equivalent to about 40 mg/day of Compound A-monohydrochloride.

In some embodiments, the titration schedule is repeated until an optimized dose is obtained. An optimized dose provides efficacy of treatment while minimizes side effects with Compound A treatment.

In some embodiments, treatment with Compound A, or a pharmaceutically acceptable salt thereof (e.g. Compound A-monohydrochloride) does not require titration. In some embodiments, an optimized dose is administered without titration.

In some embodiments, the optimized dose is equivalent to about 40 mg/day to about 160 mg/day of Compound A-monohydrochloride. In some embodiments, the optimized dose is equivalent to about 40 mg/day, about 60 mg/day, about 80 mg/day, about 100 mg/day, about 120 mg/day, about 140 mg/day, or about 160 mg/day of Compound A-monohydrochloride. In some embodiments, the optimized dose is equivalent to about 40 mg/day, about 80 mg/day, about 120 mg/day, or about 160 mg/day of Compound A-monohydrochloride. In some embodiments, the optimized dose is equivalent to about 40 mg/day of Compound A-monohydrochloride. In some embodiments, the optimized dose is equivalent to about 80 mg/day of Compound A-monohydrochloride. In some embodiments, the optimized dose is equivalent to about 120 mg/day of Compound A-monohydrochloride. In some embodiments, the optimized dose is equivalent to about 160 mg/day of Compound A-monohydrochloride.

In phase 2 clinical trials of acromegaly patients previously treated with LA-SRLs, an evaluation of steady state IGF-1 changes as a function of Compound A-monohydrochloride dose showed that 10 and 20 mg per day resulted in IGF-1 levels that were above baseline, where baseline was the IGF-1 measured at the last LA-SRL injection. The switch to Compound A-monohydrochloride occurred 4 weeks after last LA-SRL injection. Doses of 30/day and 40 mg/day resulted in changes from baseline of near zero, indicating the 30/day and 40 mg/day doses were equally effective in suppressing IGF-1 as prior monotherapy with injected LA-SRLs.

A dose-response relationship was observed when evaluating the magnitude of the rise of IGF-1 during Compound A washout in these acromegaly studies. Exposure-response modeling estimated the Compound A concentration at which 80% of maximal pharmacological response (EC80) is achieved. In some embodiments, administration of the SDD tablets described herein in an amount of about 30 mg/day to about 60 mg/day of Compound A-monohydrochloride provides daily trough concentrations that exceed EC80 and result in consistent IGF-1 suppression in patients with acromegaly.

In some embodiments, a trough concentration of Compound A required to provide therapeutic effect comparable to that of long acting SRLs in CS patients is between about 20 ng/ml to about 150 ng/mL. In some embodiments, the trough blood plasma level of Compound A required to provide therapeutic effect comparable to that of long acting SRLs in CS patients is between about 25 ng/mL to about 80 ng/mL. In some embodiments, the trough blood plasma level of Compound A required to provide therapeutic effect comparable to that of long acting SRLs in CS patients is between about 30 ng/ml to about 80 ng/mL. In some embodiments, the trough blood plasma level of Compound A required to provide therapeutic effect comparable to that of long acting SRLs in CS patients is between about 30 ng/ml to about 60 ng/ml.

In some embodiments, a trough concentration of Compound A required to provide therapeutic effect comparable to that of long acting SRLs in CS patients is at least about 20 ng/ml, at least about 25 ng/ml, at least about 30 ng/ml, at least about 35 ng/mL, at least about 40 ng/mL, or at least about 50 ng/mL. In some embodiments, paltusotine is administered at doses sufficient to provide a trough concentration of paltusotine that is at least about 20 ng/ml, at least about 21 ng/ml, at least about 22 ng/ml, at least about 23 ng/ml, at least about 24 ng/ml, at least about 25 ng/ml, at least about 26 ng/mL, at least about 27 ng/ml, at least about 28 ng/ml, at least about 29 ng/mL, at least about 30 ng/mL, at least about 31 ng/mL, at least about 32 ng/mL, at least about 33 ng/ml, at least about 34 ng/ml, at least about 35 ng/ml, at least about 36 ng/ml, at least about 37 ng/mL, at least about 38 ng/ml, at least about 39 ng/ml, at least about 40 ng/ml, at least about 41 ng/mL, at least about 42 ng/ml, at least about 43 ng/ml, at least about 44 ng/ml, at least about 45 ng/ml, at least about 46 ng/ml, at least about 47 ng/ml, at least about 48 ng/ml, at least about 49 ng/ml, at least about 50 ng/ml, at least about 51 ng/mL, at least about 52 ng/mL, at least about 53 ng/ml, at least about 54 ng/ml, at least about 55 ng/ml, at least about 56 ng/ml, at least about 57 ng/ml, at least about 58 ng/ml, at least about 59 ng/ml, at least about 60 ng/ml, at least about 61 ng/ml, at least about 62 ng/ml, at least about 63 ng/ml, at least about 64 ng/ml, at least about 65 ng/ml, at least about 66 ng/ml, at least about 67 ng/ml, at least about 68 ng/ml, at least about 69 ng/ml, at least about 70 ng/mL, at least about 71 ng/mL, at least about 72 ng/mL, at least about 73 ng/mL, at least about 74 ng/ml, at least about 75 ng/mL, at least about 76 ng/ml, at least about 77 ng/ml, at least about 78 ng/mL, at least about 79 ng/mL, at least about 80 ng/ml, at least about 81 ng/ml, at least about 82 ng/ml, at least about 83 ng/ml, at least about 84 ng/ml, at least about 85 ng/ml, at least about 86 ng/mL, at least about 87 ng/mL, at least about 88 ng/mL, at least about 89 ng/ml, at least about 90 ng/ml, at least about 91 ng/mL, at least about 92 ng/mL, at least about 93 ng/ml, at least about 94 ng/mL, at least about 95 ng/ml, at least about 96 ng/ml, at least about 97 ng/mL, at least about 98 ng/mL, at least about 99 ng/ml, or at least about 100 ng/mL.

In some embodiments, a trough concentration of Compound A required to provide therapeutic effect in CS patients is greater than 20 ng/ml, greater than 25 ng/ml, greater than 30 ng/ml, greater than 35 ng/mL, or greater than 40 ng/mL. In some embodiments, a trough concentration of Compound A required to provide therapeutic effect in CS patients is at least about 20 ng/mL, at least about 25 ng/ml, at least about 30 ng/ml, at least about 35 ng/mL, at least about 40 ng/ml, or at least about 50 ng/mL. In some embodiments, paltusotine is administered at doses sufficient to provide a trough concentration of paltusotine that is at least about 20 ng/ml, at least about 21 ng/mL, at least about 22 ng/ml, at least about 23 ng/ml, at least about 24 ng/ml, at least about 25 ng/ml, at least about 26 ng/mL, at least about 27 ng/ml, at least about 28 ng/ml, at least about 29 ng/ml, at least about 30 ng/mL, at least about 31 ng/mL, at least about 32 ng/ml, at least about 33 ng/ml, at least about 34 ng/ml, at least about 35 ng/ml, at least about 36 ng/ml, at least about 37 ng/ml, at least about 38 ng/ml, at least about 39 ng/mL, or at least about 40 ng/mL. In some embodiments, paltusotine is administered at doses sufficient to provide a trough concentration of paltusotine that is at least about 32 ng/mL.

In some embodiments, treatment of CS comprises administering a sufficient amount of Compound A to obtain a trough blood plasma level of Compound A in the CS patient that is between about 20 ng/ml to about 150 ng/mL. In some embodiments, treatment of CS comprises administering a sufficient amount of Compound A to obtain a trough blood plasma level of Compound A in the CS patient that is between about 25 ng/ml to about 80 ng/mL. In some embodiments, treatment of CS comprises administering a sufficient amount of Compound A to obtain a trough blood plasma level of Compound A in the CS patient that is between about 30 ng/mL to about 80 ng/mL. In some embodiments, treatment of CS comprises administering a sufficient amount of Compound A to obtain a trough blood plasma level of Compound A in the CS patient that is between about 30 ng/ml to about 60 ng/mL.

In some embodiments, treatment of CS comprises administering a sufficient amount of Compound A to obtain a trough blood plasma level of Compound A in the CS patient that is greater than 10 ng/ml, greater than 15 ng/mL, greater than 20 ng/ml, greater than 25 ng/ml, greater than 30 ng/mL, greater than 35 ng/mL, greater than 40 ng/ml, greater than 45 ng/ml, greater than 50 ng/mL, greater than 55 ng/ml, greater than 60 ng/mL, greater than 65 ng/ml, greater than 70 ng/ml, greater than 75 ng/mL, or greater than 80 ng/mL.

In some embodiments, treatment of CS comprises administering a sufficient amount of Compound A to obtain a trough blood plasma level of Compound A in the CS patient that is at least about 10 ng/ml, at least about 15 ng/mL, at least about 20 ng/ml, at least about 25 ng/ml, at least about 30 ng/mL, at least about 35 ng/ml, at least about 40 ng/ml, at least about 45 ng/mL, at least about 50 ng/mL, at least about 55 ng/mL, at least about 60 ng/ml, at least about 65 ng/ml, at least about 70 ng/ml, at least about 75 ng/ml, or at least about 80 ng/ml, at least about 85 ng/mL, at least about 90 ng/ml, at least about 95 ng/ml, at least about 100 ng/ml, at least about 115 ng/mL, at least about 120 ng/ml, at least about 125 ng/ml, at least about 130 ng/ml, at least about 135 ng/ml, at least about 140 ng/ml, at least about 145 ng/mL, at least about 150 ng/mL, or at least about 150 ng/mL.

In some embodiments, Compound A, or a pharmaceutically acceptable salt thereof (e.g. Compound A-monohydrochloride) is administered at doses sufficient to provide a trough concentration of Compound A that is about 20 ng/ml, about 21 ng/ml, about 22 ng/ml, about 23 ng/mL, about 24 ng/mL, about 25 ng/mL, about 26 ng/ml, about 27 ng/ml, about 28 ng/ml, about 29 ng/mL, about 30 ng/ml, about 31 ng/mL, about 32 ng/ml, about 33 ng/ml, about 34 ng/mL, about 35 ng/ml, about 36 ng/mL, about 37 ng/ml, about 38 ng/mL, about 39 ng/ml, about 40 ng/mL, about 41 ng/mL, about 42 ng/mL, about 43 ng/ml, about 44 ng/ml, about 45 ng/ml, about 46 ng/mL, about 47 ng/mL, about 48 ng/ml, about 49 ng/ml, about 50 ng/ml, about 51 ng/mL, about 52 ng/mL, about 53 ng/mL, about 54 ng/ml, about 55 ng/mL, about 56 ng/ml, about 57 ng/mL, about 58 ng/mL, about 59 ng/mL, about 60 ng/ml, about 61 ng/ml, about 62 ng/mL, about 63 ng/ml, about 64 ng/ml, about 65 ng/mL, about 66 ng/ml, about 67 ng/mL, about 68 ng/ml, about 69 ng/ml, about 70 ng/ml, about 71 ng/ml, about 72 ng/ml, about 73 ng/mL, about 74 ng/ml, about 75 ng/mL, about 76 ng/ml, about 77 ng/ml, about 78 ng/ml, about 79 ng/mL, about 80 ng/mL, about 81 ng/ml, about 82 ng/mL, about 83 ng/ml, about 84 ng/mL, about 85 ng/ml, about 86 ng/ml, about 87 ng/ml, about 88 ng/ml, about 89 ng/ml, about 90 ng/ml, about 91 ng/mL, about 92 ng/mL, about 93 ng/mL, about 94 ng/mL, about 95 ng/mL, about 96 ng/mL, about 97 ng/ml, about 98 ng/mL, about 99 ng/ml, about 100 ng/mL, or greater than about 100 ng/mL.

In some embodiments, Compound A, or a pharmaceutically acceptable salt thereof (e.g., Compound A-monohydrochloride) is administered at doses sufficient to provide a trough concentration of Compound A that is about 32 ng/mL. In some embodiments, Compound A, or a pharmaceutically acceptable salt thereof (e.g., Compound A-monohydrochloride) is administered at doses sufficient to provide a trough concentration of Compound A that is at least about 32 ng/mL. In some embodiments, Compound A, or a pharmaceutically acceptable salt thereof (e.g., Compound A-monohydrochloride) is administered at doses sufficient to provide a trough concentration of Compound A that is greater than about 32 ng/mL.

In some embodiments, once daily administration of the SDD tablets described herein to a CS patient at a total daily dose of about 40 mg to about 160 mg of Compound A-monohydrochloride provides a trough concentration of Compound A that is greater than 10 ng/ml, greater than 15 ng/ml, greater than 20 ng/mL, greater than 25 ng/mL, greater than 30 ng/ml, greater than 35 ng/ml, greater than 40 ng/mL, greater than 45 ng/ml, greater than 50 ng/mL, greater than 55 ng/mL, greater than 60 ng/mL, greater than 65 ng/mL, greater than 70 ng/mL, greater than 75 ng/mL, greater than 80 ng/mL, greater than 85 ng/ml, greater than 90 ng/ml, greater than 95 ng/ml, greater than 100 ng/ml, greater than 115 ng/mL, greater than 120 ng/ml, greater than 125 ng/mL, greater than 130 ng/mL, greater than 135 ng/mL, greater than 140 ng/mL, greater than 145 ng/ml, greater than 150 ng/ml, or greater than 150 ng/ml.

In some embodiments, once daily administration of the SDD tablets described herein to a CS patient at a total daily dose of about 40 mg to about 120 mg of Compound A-monohydrochloride provides a trough concentration of Compound A of about 10 ng/ml to about 150 ng/mL. In some embodiments, once daily administration of the SDD tablets described herein at a total daily dose of about 40 mg to about 120 mg of Compound A-monohydrochloride provides a trough concentration of Compound A of about 20 ng/mL to about 150 ng/mL.

In any of the aforementioned aspects are further embodiments comprising single administrations of the effective amount of the compound, including further embodiments in which the compound is administered once a day.

### EXAMPLES

The following examples are provided for illustrative purposes only and not to limit the scope of the claims provided herein.

### Example 1: Oral Capsules

Representative capsules are described below in Table 1.

TABLE 1

| | | Quantity per | |
|---|---|---|---|
| Component Name | Function | Unit (mg) | % w/w |
| Compound A-HCl^{a} | Drug substance | 10.00 | 5.00 |
| Microcrystalline Cellulose | Diluent | 85.5 | 42.77 |
| Mannitol | Diluent | 85.5 | 42.77 |
| Croscarmellose Sodium | Disintegrant | 7.4 | 3.70 |
| Vitamin E Polyethylene | Solubilizer | 8.5 | 4.27 |
| Glycol Succinate (TPGS) | | | |
| Colloidal Silicon Dioxide | Glidant | 2.0 | 1.00 |
| Sodium Stearyl Fumarate | Lubricant | 1.0 | 0.50 |
| Size 2 Gelatin Capsule^{c} | Capsule Shell | NA | NA |

| | | |
|---|---|---|
| Total | 200.00^{b} | 100.00 |

^{a}Amount corrected for assay and moisture, chloride and isopropyl alcohol content;
^{b}Capsule fill weight adjusted based upon blend assay;
^{c}Composed of red iron oxide, titanium dioxide and gelatin.

A representative description of the manufacturing process for the hot melt granulation capsules is as follows:

Stage 1: High Shear Wet Granulation: Melt the Vitamin E Polyethylene Glycol Succinate (TPGS). Compound A-HCl, mannitol, microcrystalline cellulose, croscarmellose sodium and silicon dioxide are charged into a high shear wet granulator and mixed. The melted Vitamin E TPGS is sprayed onto the granulation components.

Stage 2: Milling: The wet granulation is milled through a screening mill using an appropriately sized screen.

Stage 3: Blending: The sodium stearyl fumarate is sieved using an appropriately sized screen. The milled granulation is charged into the diffusion mixer (tumble) along with the sodium stearyl fumarate and blended.

Stage 4: Encapsulation: The 10 mg capsules were automatically encapsulated in Size 2 gelatin capsules.

### Example 2: Spray-Dried Solid Dispersions

Spray-dried solid dispersions were prepared with 15% by weight Compound A-HCl: 15/85 Compound A-HCl/HPMCAS-M and 15/85 Compound A-HCl/PVP VA64 formulations. The manufactures were completed using the BLD-150, the Bend Lab Dryer with 150 kg/hr drying gas capacity. The parameters varied were solution solids loading for the HPMCAS-M SDD formulation to help reduce nozzle bearding, and dryer outlet temperature for the PVP VA64 SDD to de-risk fluctuations that may occur during clinical manufacturing. The original process parameter screening plan specified manufacturing the decreased dryer outlet temperature condition with a larger orifice nozzle to produce larger particles, however based on the results of the first spray it was determined that the atomization pressure needed to achieve the desired solution flow rate would have been too low to fully atomize the solution with the larger orifice. As dryer outlet temperature tends to have more variability than solution flow rate, the parameter screen was shifted to focus on de-risking the dryer outlet temperature alone while ensuring fully atomized droplets. Dryer outlet temperature variation could affect residual solvent levels in the SDDs, which can affect physical and chemical stability. All sprays were completed successfully with good yields, and indicate a robust processing space for both formulations.

### 15/85 Compound A HPMCAS-M SDD Formulation Manufacturing Details

Three sub batches of 15/85 Compound A-HCl/HPMCAS-M SDDs were sprayed to explore the manufacture processing space and prepare for clinical trial manufacturing. A sub batch was sprayed first at 10 wt % solids, and significant nozzle bearding that appeared to affect the spray plume was observed after about 45 minutes on solution.

The solution was diluted to 8 wt %, and a sub batch was manufactured with a duration of 1 hour to ensure bearding was reduced. A very small amount of bearding was observed during this batch after about 50 minutes on solution, but did not appear to affect the atomization plume and the 8 wt % solids loading was selected.

Cooling water at 2 GPM and approximately 7° C. was run through the spray dryer lid throughout all sprays to keep the lid cool and prevent sticking and browning. No significant lid buildup or browning was seen throughout manufacturing. No cleaning was performed between sprays, and all sprays were completed from one solution with additional solvent added prior to manufacturing batches 2A and 2C. A summary of the manufacturing parameters used for all three sub batches is shown in Table 2.

TABLE 2
Manufacturing summary for 15/85 Compound A-HCl/HPMCAS-M SDDs.

| Parameter | Lot 2A | Lot 2B | Lot 2C |
|---|---|---|---|
| Bulk SDD collected (g) | 5316 | 2231 | 624 |
| Solids loading in solution (wt %) | 8 | 10 | 8 |
| Solvent | Methanol | | |
| Atomizer | Pressure swirl: Steinen A75 | | |
| Atomizing pressure (psig) | 370 | 301 | 345 |
| Solution flow (g/min) | 131 | 132 | 131 |
| Drying gas flow rate (g/min) | 1848 | 1848 | 1848 |
| Dryer inlet temperature (° C.) | 146 | 145 | 144 |
| Dryer outlet temperature (° C.) | 45 | 45 | 45 |
| Bulk secondary tray drying | 16 hours at 40° C./15% RH | 17 hours at 40° C./15% RH | |
| Dry yield (%)¹ | 96 | 96 | 93 |
| Spray duration (hours) | 8 | 3 | 1 |

| | |
|---|---|
| Calculated dryer relative saturation (%) | 11 |
| Calculated dew point (° C.) | 3 |

¹Dry yield is calculated as SDD samples and dry bulk collected divided by the amount of solids sprayed.

### 15/85 Compound A-HCl/PVP VA64 SDD Formulation Manufacturing Details

Process parameter screening sprays and an FPN demonstration batch were also completed for the 15/85 Compound A-HCl/PVP VA64 SDD formulation. The dryer outlet temperature was varied to de-risk process parameter variability to prepare for clinical trial manufacturing.

The process space was constrained by a maximum dryer outlet temperature, the dryer outlet temperature, and the minimum desired solution flow rate. A maximum inlet temperature of 160° C. was specified to avoid sticking or browning of SDD on the spray dryer lid, and the minimum flow rate was set at 100 g/min to ensure sufficient throughput. The minimum and maximum dryer outlet temperatures were chosen to be 40° C. and 65° C. respectively to ensure adequate particle drying and that the dryer outlet temperature will not be above the wet particle Tg.

The process parameter screening sprays for the PVP VA64 formulation explored the manufacturing space by varying the dryer outlet temperature. This allowed investigation of the effect of dryer relative saturation on particle residual solvent content, morphology, density, and stability.

Cooling water was run at 2 GPM and approximately 7° C. throughout all sprays to prevent lid buildup and browning, and neither were noted. No nozzle bearding was observed throughout all three manufactures. All sprays were completed from one solution. The manufacturing details of each sub batch are summarized in Table 3.

TABLE 3
15/85 Compound A-HCl*/PVP VA64 SDD process parameters

| Parameter | Lot 4A | Lot 4B | Lot 4C |
|---|---|---|---|
| Bulk SDD collected (g) | 5285 | 623 | 624 |
| Solids loading in solution (wt %) | 8 | | |
| Solvent | Methanol | | |
| Atomizer | Pressure swirl: Steinen A75 | | |
| Atomizing pressure (psig) | 422 | 449 | 445 |
| Solution flow (g/min) | 128 | 133 | 132 |
| Drying gas flow rate (g/min) | 1845 | 1848 | 1849 |
| Dryer inlet temperature (° C.) | 143 | 153 | 132 |
| Dryer outlet temperature (° C.) | 45 | 49 | 40 |
| Bulk secondary tray drying | 18 hours at 40° C./15% RH | 26 hours at 40° C./15% RH | |
| Dry yield (%)¹ | 92 | 86 | 91 |
| Spray duration (hours) | 8 | 1 | 1 |
| Calculated dryer relative saturation (%) | 11 | 9 | 14 |

| | |
|---|---|
| Calculated dew point (° C.) | 3 |

*Compound A-HCl, formulated on a basis of free base.
¹Dry yield is calculated as SDD samples and dry bulk collected divided by the amount of solids sprayed.

### SDD Characterization

Particle Properties: The particle size distribution and bulk and tapped densities were measured for each batch of Compound A-HCl SDD. The HPMCAS-M SDDs have larger particle sizes, which is expected because the HPMCAS-M solution is more viscous than the PVP VA64 solution which leads to larger droplets for a given nozzle configuration. The increased solids loading in solution of lot 2B led to larger particles than batches 2A and 2C, which is also due to the higher viscosity of the spray solution. The particle size distributions of all PVPVA-64 batches are similar, as expected.

The bulk and tapped densities of 2A and 2C are similar, while batch 2B has a slightly lower density potentially due to the larger particles. The bulk and tapped densities of all PVPVA-64 batches are similar, indicating a robust process with respect to the dryer outlet temperature effects on powder properties.

Residual Solvent and Water Content: The residual methanol and water of the six SDDs were measured using GC and KF respectively. All SDDs contained residual methanol below the ICH guideline of 0.3 wt % after secondary drying, suggesting adequate drying at 40° C./15% RH.

Morphology by SEM: The particle morphology of all six SDDs was evaluated via SEM. Each SDD showed typical morphology with no evidence of irregular particles suggesting adequate atomization for all conditions tested. The HPMCAS-M particles were primarily collapsed spheres while the PVP VA64 SDDs contain a larger fraction of spherical particles.

Crystallinity by PXRD: All six SDDs were evaluated for crystallinity using PXRD. All SDDs were amorphous by PXRD as evident in the absence of sharp diffraction peaks.

Thermal Characteristics by DSC: All six SDDs were characterized by modulated DSC. The results are tabulated in Table 4. The manufactured SDDs were all amorphous and homogenous by DSC as evident by the presence of a single glass transition in the reversing heat signal. Neither formulation shows signs of crystallization after the Tg suggesting low propensity for crystallization of Compound A at those temperatures for both formulations. Furthermore, both formulations showed high Tg relative to ambient temperatures suggesting low physical stability risks in dry conditions. Packaging to minimize humidity will be necessary for the PVP VA64 formulation.

TABLE 4
Tabulated thermal characteristics of the six batches as measured by DSC.

| | | | | Delta Cp (J/ | |
|---|---|---|---|---|---|
| Sample | Lot | Tg (° C.) | Std Dev* | (g*° C.)) | Std Dev* |
| 15/85 Compound A- | 2A | 135.2 | 0.35 | 0.27 | 0.03 |
| HCl/HPMCAS-M SDD | 2B | 134.7 | 0.36 | 0.25 | 0.02 |
| | 2C* | 134.5 | 0.95 | 0.26 | 0.00 |
| 15/85 Compound A-HCl/ | 4A* | 125.5 | 0.04 | 0.31 | 0.01 |
| PVP VA64 SDD | 4B | 125.6 | 0.26 | 0.30 | 0.01 |
| | 4C | 125.6 | 0.17 | 0.35 | 0.08 |

*Average is from n = 2 replicates. As a result, std deviation is actually calculated as range/2.

### SUMMARY

Physical Stability observations: PVP VA64 SDD appeared to deliquesce upon storage with crystals observed at 3 months (40° C./75% RH open). Storage with desiccant is recommended. HPMCAS-M SDD was physically stable through 6 months at 40° C./75% RH open.

Chemical Stability observations: Possible acid catalyzed degradation in the HPMCAS-M formulation on stability. Some degradation in the PVP VA64 formulation as well, but not as significant as the HPMCAS-M SDD. Packaging will be required for the PVPVA SDD which will be driven by physical stability concerns.

The 15% by weight Compound A/PVP VA64 was selected as the lead SDD formulation.

### 12-Month Stability: 15% Compound A-HCl/PVP-VA64 SDD

12-month SDD samples were held at 5° C., 25° C./60% RH, and 40° C./75% RH with desiccant. Samples for water analysis by Karl Fisher titration were prepared and analyzed immediately; the remaining samples were vacuum desiccated overnight to remove residual moisture and preserve the physical state of SDD's for further characterization. A list of the analytical tests performed for characterization included: appearance, water content by Karl Fisher titration, Powder x-ray diffraction (PXRD), scanning electron microscopy (SEM), thermal characterization by modulated differential scanning calorimetry (mDSC), dissolution performance by micro-centrifuge (MCT) test, assay and related substances by HPLC.

Conclusion from PXRD analysis on the 12-month SDD stability samples: There was no evidence of crystallinity in samples stored at 12 months at each of the stability conditions.

Conclusion from SEM analysis on the 12-month SDD stability samples: No particle fusion was observed across all stability conditions at 12 month. There was no evidence of crystallinity in samples stored at 12 months at each of the stability conditions.

Conclusion from SEM analysis on the 12-month SDD stability samples: No particle fusion is observed across all stability conditions at 12 months. There was no evidence of crystallinity in samples stored at 12 months at each of the stability conditions.

Conclusions from mDSC analysis on the 12-month SDD stability samples: Repeated analysis of the 12 month SDD sample held at 5° C. affords non-reproducible thermograms, the cause for this result is not known at this time. The 5° C. sample was determined to be physically stable by all other characterization techniques. The 12 month SDD samples held at 25° C./60% RH and 40° C./75% RH showed a single, reproducible Tg at 124-125° C., supporting the conclusion that the SDD is stable after 12 months of storage with desiccant at these conditions.

Conclusion from MCT dissolution analysis on the 12-month SDD stability samples: Non-sink dissolution performance of the 12 month stability samples is consistent with the initial (to) sample stored at −20° C.

### 35/65 Compound A-HCl/PVP VA64 SDD Formulation Manufacturing Details

Spray-dried solid dispersions were prepared with 35% by weight Compound A-HCl: 35/65 Compound A-HCl/PVP VA64 formulation.

The manufacture of the SDD was completed using the SD-180 lab dryer. Secondary drying was completed using the Binder Convection Dryer. The manufacturing details are summarized in Table 5.

The spray was completed successfully with good yield.

TABLE 5
35/65 Compound A-HCl*/PVP VA64 SDD process parameters

| Parameter | Value |
|---|---|
| Bulk SDD collected (g) | 2796 |
| Solids loading in solution (wt %) | 10.8 |
| Solvent | Methanol |
| Atomizer | SD-90 with Pressure Swirl |
| Atomizing pressure (psig) | 320 |
| Solution flow (g/min) | 115 |
| Drying gas rate (acfm) | 80 |
| Dryer inlet temperature (° C.) | 100 |
| Dryer outlet temperature (° C.) | 45 |
| Condensore Setpoint (° C.) | −20 |
| Bulk secondary tray drying | 48 hours at 40° C./ambient pressure |

*Compound A-HCl, formulated on a basis of free base

### Example 3: Oral Tablets

Representative 10 mg, 20 mg, 30 mg, 40 mg, and 60 mg spray-dried dispersion tablets are presented in Tables 6, 7, 8, 9, 10, 11, 12, and 13.

Typical excipients used to prepare the tablets included: microcrystalline cellulose, mannitol, crospovidone, colloidal silicon dioxide, magnesium stearate, and Opadry White 03K184116 (film coating).

TABLE 6
Representative 10 mg spray-dried dispersion tablets.

| | | | |
|---|---|---|---|
| | | Quantity per | |
| Component Name | Function | Unit (mg) | % w/w |

Spray-Dried Dispersion

| | | | |
|---|---|---|---|
| Compound A-HCl | Drug substance | 10.00 | 3.20 |
| PVP/VA 64 | Film Forming | 56.67 | 18.15 |
| | Agent | | |
| Methanol^{a} | Solvent | NA | NA |

Roller Compaction/Blending

| | | | |
|---|---|---|---|
| Microcrystalline Cellulose | Diluent | 130.31 | 41.74 |
| Mannitol | Diluent | 53.04 | 16.99 |
| Crospovidone | Disintegrant | 30.29 | 9.71 |
| Colloidal Silicon Dioxide | Glidant | 1.53 | 0.49 |
| Magnesium Stearate | Lubricant | 0.76 | 0.24 |

Blending/Compression

| | | | |
|---|---|---|---|
| Crospovidone | Disintegrant | 18.18 | 5.82 |
| Colloidal Silicon Dioxide | Glidant | 1.52 | 0.49 |
| Magnesium Stearate | Lubricant | 0.76 | 0.24 |

Film Coating

| | | | |
|---|---|---|---|
| Opadry ® White 03K18416^{b} | Film Coating | 9.09 | 2.91 |
| | Agent | | |
| Purified Water^{c} | Solvent | NA | NA |

| | | |
|---|---|---|
| Total | 312.15 | 100.0 |

^{a}Methanol removed on drying during the spray drying process.
^{b}Composed of Hypromellose 2910, titanium dioxide and triacetin.
^{c}Purified water removed on drying during the film coating process.

TABLE 7
Representative 20 mg spray-dried dispersion (HPMCAS-M) tablets.

| Formulation no. | | |
|---|---|---|
| A1 | A2 | A3 |
| Tablet Dose/Tablet Weight (mg/mg) | | |
| 20/400 | 20/400 | 20/400 |

| | | |
|---|---|---|
| Function | Ingredient | % w/w of Blend |

Pregranulation

| | | | | |
|---|---|---|---|---|
| Active | 15/85 Compound A-HCl/HPMCAS-M | 33.33% | 33.33% | 33.33% |
| Local PH Modifier | Citric Acid | — | — | 5.00% |
| Filler | Microcrystalline Cellulose (Avicel PH-102) | 38.11% | 40.11% | 36.78% |
| Filler | Mannitol (Mannogem EZ Spray Dried) | 19.06% | 20.06% | 18.39% |
| Disintegration Aid | Sodium Chloride (NaCl Powder) | — | — | — |
| Disintegrant | Sodium Starch Glycolate (Explotab) | 5.00% | — | — |
| Disintegrant | Crospovidone (PVP-XL) | — | 5.00% | 3.00% |
| Glidant | Silicon dioxide (Syloid 244 FP) | 0.50% | 0.50% | 0.50% |
| Lubricant | Magnesium Stearate | 0.25% | 0.25% | 0.25% |

Extragranular

| | | | | |
|---|---|---|---|---|
| Disintegrant | Sodium Starch Glycolate (Explotab) | 3.00% | — | — |
| Disintegrant | Crospovidone (PVP-XL) | — | — | 2.00% |
| Glidant | Silicon dioxide (Syloid 244 FP) | 0.50% | 0.50% | 0.50% |
| Lubricant | Magnesium Stearate | 0.25% | 0.25% | 0.25% |

TABLE 8
Representative 20 mg spray-dried dispersion (PVPVA 64) tablets.

| Formulation no. | | |
|---|---|---|
| B1 | B2 | B3 |
| Tablet Dose/Tablet Weight (mg/mg) | | |
| 20/400 | 20/400 | 20/400 |

| | | |
|---|---|---|
| Function | Ingredient | % w/w of Blend |

Pregranulation

| | | | | |
|---|---|---|---|---|
| Active | 15/85 Compound A-HCl/PVPVA 64 | 33.33% | 33.33% | 33.33% |
| Local PH Modifier | Citric Acid | 5.00% | — | — |
| Filler | Microcrystalline Cellulose (Avicel PH-102) | 43.17% | 33.17% | 33.17% |
| Filler | Mannitol (Mannogem EZ Spray Dried) | — | 20.00% | — |
| Disintegration Aid | Sodium Chloride (NaCl Powder) | 5.00% | — | 20.00% |
| Disintegrant | Sodium Starch Glycolate (Explotab) | 6.00% | — | — |
| Disintegrant | Crospovidone (PVP-XL) | — | 6.00% | 6.00% |
| Glidant | Silicon dioxide (Syloid 244 FP) | 0.50% | 0.50% | 0.50% |
| Lubricant | Magnesium Stearate | 0.25% | 0.25% | 0.25% |

Extragranular

| | | | | |
|---|---|---|---|---|
| Disintegrant | Sodium Starch Glycolate (Explotab) | 6.00% | — | — |
| Disintegrant | Crospovidone (PVP-XL) | — | 6.00% | 6.00% |
| Glidant | Silicon dioxide (Syloid 244 FP) | 0.50% | 0.50% | 0.50% |
| Lubricant | Magnesium Stearate | 0.25% | 0.25% | 0.25% |

TABLE 9
Additional representative 20 mg spray dried dispersion tablets.

| Formulation no. | | | |
|---|---|---|---|
| A4 | B4 | B5 | B6 |
| Tablet Dose/Tablet Weight (mg/mg) | | | |
| 20/400 | 20/606.1 | 20/400 | 20/606.1 |

| | | |
|---|---|---|
| Function | Ingredient | % w/w of Blend |

Pregranulation

| | | | | | |
|---|---|---|---|---|---|
| Active | 15/85 Compound A-HCl/HPMCAS-M | 33.33% | — | — | — |
| Active | 15/85 Compound A-HCl/PVPVA 64 | — | 22.00% | 33.33% | 22.00% |
| Local PH Modifier | Citric Acid | 5.00% | 5.00% | — | — |
| Filler | Microcrystalline Cellulose (Avicel PH-102) | 35.61% | 40.50% | 33.17% | 43.00% |
| Filler | Mannitol (Mannogem EZ Spray Dried) | 16.56% | 15.00% | 16.00% | 17.50% |
| Disintegrant | Crospovidone (PVP-XL) | 5.00% | 10.00% | 10.00% | 10.00% |
| Glidant | Silicon dioxide (Syloid 244 FP) | 0.50% | 0.50% | 0.50% | 0.50% |
| Lubricant | Magnesium Stearate | 0.25% | 0.25% | 0.25% | 0.25% |

Extragranular

| | | | | | |
|---|---|---|---|---|---|
| Disintegrant | Crospovidone (PVP-XL) | 3.00% | 6.00% | 6.00% | 6.00% |
| Glidant | Silicon dioxide (Syloid 244 FP) | 0.50% | 0.50% | 0.50% | 0.50% |
| Lubricant | Magnesium Stearate | 0.25% | 0.25% | 0.25% | 0.25% |

TABLE 10
Representative 30 mg and 40 mg spray-dried dispersion (PVPVA 64) tablets.

| | | Formulation no. | |
|---|---|---|---|
| | | D1 | D2 |
| | | Tablet Dose/Tablet Weight (mg/mg) | |
| | | 30/909.2 | 40/1212.3 |
| Function | Ingredient | % w/w of Blend | |
| Active | 15/85 Compound A-HCl/PVPVA 64 | 22.00 | 22.00 |
| Filler | Microcrystalline Cellulose (Avicel PH-101) | 43.00 | 43.00 |
| Filler | Mannitol (Parteck M100) | 17.50 | 17.50 |
| Disintegrant | Crospovidone (PVP-XL) | 10.00 | 10.00 |
| Glidant | Silicon dioxide (Syloid 244 FP) | 0.50 | 0.50 |
| Lubricant | Magnesium Stearate | 0.25 | 0.25 |
| Disintegrant | Crospovidone (PVP-XL) | 6.00 | 6.00 |
| Glidant | Silicon dioxide (Syloid 244 FP) | 0.50 | 0.50 |
| Lubricant | Magnesium Stearate | 0.25 | 0.25 |

TABLE 11
Additional representative 40 mg spray-dried dispersion (PVPVA 64) tablets.

| Formulation no. | | |
|---|---|---|
| E1 | E2 | E3 |
| Tablet Dose/Tablet Weight (mg/mg) | | |
| 40/500 | 40/600 | 40/600 |

| | | |
|---|---|---|
| Function | Ingredient | % w/w of Blend |

Pregranulation

| | | | | |
|---|---|---|---|---|
| Active | 35/65 Compound A-HCl/PVPVA 64 | 34.29% | 28.57% | 28.57% |
| Filler | Microcrystalline Cellulose (Avicel PH-101) | 32.14% | 28.18% | 22.18% |
| Filler | Mannitol (Parteck M100) | 16.07% | 14.09% | 11.09% |
| Disintegrant | Croscarmellose Sodium (Ac-Di-Sol) | 5.00% | 5.00% | 7.81% |
| Disintegrant | Crospovidone (PVP-XL) | 5.00% | 5.00% | 7.81% |
| Glidant | Silicon dioxide (Syloid 244 FP) | 0.50% | 0.50% | 0.50% |
| Lubricant | Magnesium Stearate | 0.25% | 0.25% | 0.25% |

Extragranular

| | | | | |
|---|---|---|---|---|
| Filler | Microcrystalline Cellulose (Avicel PH-101) | — | 11.67% | 11.67% |
| Disintegrant | Croscarmellose Sodium (Ac-Di-Sol) | 3.00% | 3.00% | 4.69% |
| Disintegrant | Crospovidone (PVP-XL) | 3.00% | 3.00% | 4.69% |
| Glidant | Silicon dioxide (Syloid 244 FP) | 0.50% | 0.50% | 0.50% |
| Lubricant | Magnesium Stearate | 0.25% | 0.25% | 0.25% |

TABLE 12
Representative 60 mg spray-dried dispersion (PVPVA 64) tablets.

| Formulation no. | | |
|---|---|---|
| C1 | C2 | C3 |
| Tablet Dose/Tablet Weight (mg/mg) | | |
| 60/500 | 60/600 | 60/600 |

| | | |
|---|---|---|
| Function | Ingredient | % w/w of Blend |

Pregranulation

| | | | | |
|---|---|---|---|---|
| Active | 35/65 Compound A-HCl/PVPVA 64 | 34.29% | 28.57% | 28.57% |
| Filler | Microcrystalline Cellulose (Avicel PH-101) | 32.14% | 28.18% | 22.18% |
| Filler | Mannitol (Parteck M100) | 16.07% | 14.09% | 11.09% |
| Disintegrant | Croscarmellose Sodium (Ac-Di-Sol) | 5.00% | 5.00% | 7.81% |
| Disintegrant | Crospovidone (PVP-XL) | 5.00% | 5.00% | 7.81% |
| Glidant | Silicon dioxide (Syloid 244 FP) | 0.50% | 0.50% | 0.50% |
| Lubricant | Magnesium Stearate | 0.25% | 0.25% | 0.25% |

Extragranular

| | | | | |
|---|---|---|---|---|
| Filler | Microcrystalline Cellulose (Avicel PH-101) | — | 11.67% | 11.67% |
| Disintegrant | Croscarmellose Sodium (Ac-Di-Sol) | 3.00% | 3.00% | 4.69% |
| Disintegrant | Crospovidone (PVP-XL) | 3.00% | 3.00% | 4.69% |
| Glidant | Silicon dioxide (Syloid 244 FP) | 0.50% | 0.50% | 0.50% |
| Lubricant | Magnesium Stearate | 0.25% | 0.25% | 0.25% |

TABLE 13
Additional representative 60 mg spray-dried dispersion (PVPVA 64) tablets.

| Formulation no. | | |
|---|---|---|
| C4 | C5 | C6 |
| Tablet Dose/Tablet Weight (mg/mg) | | |
| 60/600 | 60/600 | 60/600 |

| | | |
|---|---|---|
| Function | Ingredient | % w/w of Blend |

Pregranulation

| | | | | |
|---|---|---|---|---|
| Active | 35/65 Compound A-HCl/PVPVA 64 | 28.57% | 28.57% | 28.57% |
| Filler | Microcrystalline Cellulose (Avicel PH-101) | 15.08% | 18.41% | 18.41% |
| Filler | Mannitol (Parteck M100) | 7.54% | 9.21% | 9.21% |
| Disintegrant | Pregelatinized Starch (Starch 1500) | 20.00% | — | — |
| Disintegration Aid | Sodium Chloride (NaCl Powder) | — | 10.00% | — |
| Disintegration Aid | 1:1 Sodium Chloride:Potassium Chloride | — | — | 10.00% |
| Disintegrant | Croscarmellose Sodium (Ac-Di-Sol) | 5.00% | 5.00% | 5.00% |
| Disintegrant | Crospovidone (PVP-XL) | 5.00% | 5.00% | 5.00% |
| Glidant | Silicon dioxide (Syloid 244 FP) | 0.50% | 0.50% | 0.50% |
| Lubricant | Magnesium Stearate | 0.25% | 0.25% | 0.25% |

Extragranular

| | | | | |
|---|---|---|---|---|
| Filler | Microcrystalline Cellulose (Avicel PH-101) | 11.31% | 11.31% | 11.31% |
| Disintegrant | Croscarmellose Sodium (Ac-Di-Sol) | 3.00% | 3.00% | 3.00% |
| Disintegrant | Crospovidone (PVP-XL) | 3.00% | 3.00% | 3.00% |
| Glidant | Silicon dioxide (Syloid 244 FP) | 0.50% | 0.50% | 0.50% |
| Disintegration Aid | Sodium Chloride (NaCl Powder) | — | 5.00% | — |
| Disintegration Aid | 1:1 Sodium Chloride:Potassium Chloride | — | — | 5.00% |
| Lubricant | Magnesium Stearate | 0.25% | 0.25% | 0.25% |

TABLE 14
Exemplary spray-dried dispersion tablets.

| | | |
|---|---|---|
| Function | Exemplary Ingredients | % w/w of Tablet |

Pregranulation

| | | |
|---|---|---|
| Active (SDD) | 15/85 Compound A-HCl/HPMCAS-M, | 20 to 35% |
| | 15/85 Compound A-HCl/PVPVA 64, or | |
| | 35/65 Compound A-HCl/PVPVA 64 | |
| Filler(s) | Microcrystalline Cellulose, | 20 to 60% |
| | Mannitol | |
| Disintegrant(s) | Pregelatinized Starch, | 5 to 30% |
| | Croscarmellose Sodium, | |
| | Crospovidone | |
| Disintegration Aid(s) | Sodium Chloride (NaCl Powder), | 0 to 10% |
| | 1:1 Sodium Chloride:Potassium Chloride | |
| Glidant | Silicon dioxide | 0.25 to 1% |
| Lubricant | Magnesium Stearate | 0.25 to 1% |

| | |
|---|---|
| Total Pregranulation | 70% to 85% |

Extragranular

| | | |
|---|---|---|
| Filler(s) | Microcrystalline Cellulose | 0 to 20% |
| Disintegrant(s) | Croscarmellose Sodium, | 3 to 10% |
| | Crospovidone | |
| Disintegration Aid(s) | Sodium Chloride (NaCl Powder), | 0 to 5% |
| | 1:1 Sodium Chloride:Potassium Chloride | |
| Glidant | Silicon dioxide | 0.25 to 1% |
| Lubricant | Magnesium Stearate | 0.25 to 1% |

| | |
|---|---|
| Total Extragranular | 15 to 30% |

A representative non-limiting description of the manufacturing process for the SDD tablets is as follows:

Stage 1: Spray Drying: Compound A-HCl and copovidone are dissolved in MeOH. The solution spray-dried. Spray-dried dispersion (Compound A-HCl SDD) is collected.

Stage 2: Roller Compaction: Granulation blend consisting of Compound A-HCl SDD, filler(s), disintegrant(s), glidant(s), and lubricant(s) are blended. In some embodiments, granulation blend consisting of Compound A-HCl SDD, mannitol, microcrystalline cellulose, crospovidone, colloidal silicon dioxide are prepared and blended. The intra-granular portion of the magnesium stearate is screened and added to the granulation blend. The resulting blend is blended. Granulation blend is charged into hopper of the roller compaction and compacted into ribbons. The ribbons are passed through mesh screen using in-line oscillating mill to break up the ribbons and mill into granules.

In some embodiments, the granulation blend comprises about 20% to about 35% (w/w of the final tablet weight) Compound A-HCl SDD. In some embodiments, the granulation blend comprises about 21%, about 22%, about 28%, 29%, about 33%, about 34% (w/w of the final tablet weight) Compound A-HCl SDD. In some embodiments, the Compound A-HCl SDD comprises a 15/85 Compound A-HCl/HPMCAS-M, 15/85 Compound A-HCl/PVPVA64, or 35/65 Compound A-HCl/PVPVA 64 SDD.

Stage 3: Blending: The intra-granular material is mixed with the extragranular excipients. Extragranular excipients comprise one or more excipients selected from: fillers, disintegrants, glidants, and lubricants. The extragranular components include microcrystalline cellulose, crospovidone, colloidal silicon dioxide. The extragranular lubricant, magnesium stearate, is sieved using an appropriately sized screen, then added to the blend and mixed.

Stage 4: Compression: The final blend is compressed into tablets.

Stage 5: Pan-Coating: Film coating suspension of Opadry White 03K18416. is prepared in purified water, and tablets are coated with Opadry White 03K18416 in a perforated coating pan.

### Example 4: Evaluation of Formulation Performance in Dogs

### Study Designs

Evaluated two conditions* in the dog: +Pg pretreatment (mimics human fasted stomach, pH 1-2) and −Pg pretreatment (mimics humans taking PPIs or antacids, pH 3-5). (*: 1-week washout between each condition; Pg=pentagastrin.)

### Compound A-HCl Solution

N=4 non-naïve dog. Vehicle: propylene glycol. Conditions: −Pg.

### Compound A-HCl HMG Capsule

N=4 non-naïve dog. Conditions: +Pg, −Pg.

### Compound A-HCl Spray-Dried Dispersion Tablets: PVPVA

2 groups of N=6 non-naïve male dogs. Conditions: +Pg, −Pg.

Results from this study is presented in Tables 15 and 16.

TABLE 15
Dog PK evaluation of formulations of Compound A-HCl

| | | 20 mg HMG capsule | | 20 mg PVPVA SDD tablets | |
|---|---|---|---|---|---|
| PK | Solution | Fasted | Fasted | Fasted | Fasted |
| Parameters | (−Pg) | +Pg | −Pg | +Pg | −Pg |
| Cₘₐₓ (ng/mL) | 191 ± 55.2 | 126 ± 99.6 | 14.2 ± 2.9 | 67.5 ± 41.3 | 125 ± 72.6 |
| AUC₀₋ₜ (ng*hr/mL) | 1390 ± 559 | 874 ± 623 | 85.2 ± 32.6 | 366 ± 243 | 454 ± 226 |

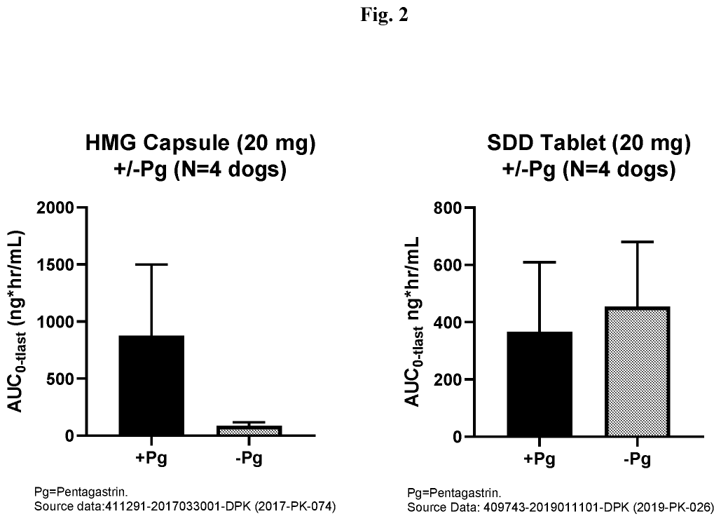

As shown in Table 15 and FIG. 2, the HMG capsule formulations performed poorly in dogs not pretreated with pentagastrin, whereas the spray-dried dispersions tablets performed better; For HMG capsule formulation, AUC without pentagastrin was only 11% of the AUC with pentagastrin (98.2 ng*hr/mL compared to 917 ng*hr/mL). In comparison, the AUC for the PVPVA SDD tablet formulations without pentagastrin was 185% and 124% of with pentagastrin condition, respectively. These data show that PVPVA SDD tablet formulations are superior under high gastric pH environment (e.g., as would be in subjects that are taking PPI or antacids).

TABLE 16
Dog PK evaluation of 60 mg 35/65 PVP-VA
SDD tablet of Compound A-HCl

| PK | 35% SDD | 60 mg PVPVA SDD tablet | | |
|---|---|---|---|---|
| Parameters | Suspension | Tablet C3 | Tablet C4 | Tablet C5 |
| Cₘₐₓ (ng/mL) | 437 ± 140 | 377 ± 129 | 329 ± 144 | 357 ± 146 |
| AUC₀₋ₜ | 3630 ± 1990 | 3110 ± 1360 | 2540 ± 1120 | 2560 ± 1140 |
| (ng*hr/mL) | | | | |

### Example 5: A Phase 1, Multi-Cohort, Single Dose Study to Assess the Relative Bioavailability, Performance, and Safety of Two Formulations of Compound A

The study was conducted in up to 3 cohorts, each with a specific primary objective:

Cohort 1: To characterize performance of 10 mg tablets prepared by spray-dried dispersion (SDD) of Compound A-HCl salt.

Cohort 2: To evaluate the relative bioavailability of 10 mg SDD tablets compared to the Compound A-HCl hot melt granulation (HMG) formulation, 10 mg capsules. To determine the effect of timing of food administration on pharmacokinetics of low dose of the 10 mg SDD tablets.

Cohort 3: To determine the effect of timing of food administration on pharmacokinetics of SDD tablets and dose proportionality in doses higher than 20 mg. To determine the optimal dosing regimen that results in adequate systemic exposure with short post-dose fasting duration.

Cohort 4: To determine the effect of timing of food administration on pharmacokinetics of SDD tablets in doses higher than 20 mg. To determine the optimal dosing regimen of paltusotine that results in high systemic exposure with low post-dose fasting duration

Cohort 5: To determine the effect of the proton-pump inhibitor (PPI), lansoprazole, on the pharmacokinetics of a 60 mg dose of SDD tablets. To determine the effect of a low-fat meal on the pharmacokinetics of a 60 mg dose of SDD tablets.

### Study Design:

Up to thirty-six (36) healthy male and female subjects were enrolled. Cohorts 1-2 consisted of four periods each, and Cohort 3 consisted of three periods.

### Cohort 1:

The SDD tablets were evaluated. Up to twelve (12) healthy male and female subjects were enrolled in each cohort. Cohort 1 consisted of 4 periods: In Period 1, subjects were administered a proton-pump inhibitor (lansoprazole, 15 mg BID for 3 days (from Day-3), taken orally at least 30 min prior to a meal, once in the morning and once in the evening). On the fourth day (Day 1 of study), fasted subjects will take the last dose of lansoprazole (15 mg) 60 min prior to 20 mg Compound A (2×10 mg of the SDD tablets). In Period 2, fasted subjects were administered 20 mg Compound A (2×10 mg of the SDD tablets). In Period 3, fasted subjects were administered 20 mg Compound A (2×10 mg of the SDD tablets) with a high-fat, high-calorie meal. In Period 4, fasted subjects will take up to 80 mg Compound A (up to 8×10 mg of the SDD tablets). The actual dose was selected based on the pharmacokinetic data from Period 2.

For Period 1: In the evening before dosing (Day −1), subjects were administered their evening dose of 15 mg lansoprazole, provided an evening meal at least 30 min after administration of lansoprazole, and then were required to fast overnight (≥10 hr) on Day −1. On Day 1, they were administered the morning dose (last dose) of 15 mg lansoprazole at least 60 min prior to administration of Compound A (2×10 mg SDD tablets). Subjects continued to fast for 2 hr after Compound A, after which they were allowed to ingest a standard meal.

For Period 2: Subjects were required to fast overnight (≥10 hr) on Day 7. On Day 8, 20 mg Compound A (2×10 mg SDD tablets) was administered orally. Subjects continued to fast for 2 hr after Compound A, after which they were allowed to ingest a standard meal.

For Period 3: Subjects were required to fast overnight (≥10 hr) on Day 14. On Day 15, they were allowed to ingest a high-fat, high-calorie meal within 30 min. Upon completion of the ingestion of the meal, Compound A (2×10 mg SDD tablets) was administered (no more than 30 min after the start of the meal). No additional food was provided for at least 4 hr after administration of Compound A.

Subjects were not allowed to perform strenuous exercise of >30 min/day 3 days prior to Day −1 and throughout the study.

PK and safety assessments including adverse event (AE) monitoring, clinical laboratory tests, vital sign measurements, 12-lead ECGs, Holter and telemetry monitoring (Period 4 only), and physical examinations were conducted at scheduled times throughout the study.

### Cohort 2:

The cohort consisted of four periods. In each period, a single dose of 20 mg Compound A (2×10 mg SDD) was administered orally.

For Period 1: Subjects were required to fast overnight (≥10 hr) on Day −1. On Day 1, a low-fat meal was given 2 hours after administration of 20 mg Compound A (2×10 mg HMG capsules; reference formulation).

For Period 2: Subjects were required to fast overnight (≥10 hr) on Day 7. On Day 8, they were given a low-fat meal 2 hr after administration of 20 mg Compound A (2×10 mg SDD tablets; test formulation).

For Period 3: Subjects were required to fast overnight (≥10 hr) on Day 14. On Day 15, they were given a low-fat meal 1 hr after administration of 20 mg Compound A (2×10 mg SDD tablets).

For Period 4: Subjects were required to fast overnight (≥10 hr) on Day 21. On Day 22, they were given a low-fat meal 0.5 hr after administration of 20 mg Compound A (2×10 mg SDD tablets).

The final study visit occurred on Day 29. Subjects were not allowed to perform strenuous exercise of >30 min/day, 3 days prior to Day −1 and throughout the study. PK and safety assessments including adverse event (AE) monitoring, clinical laboratory tests, vital sign measurements, 12-lead ECGs, and physical examinations were conducted at scheduled times throughout the study.

### Cohort 3:

The cohort consisted of three periods. In each period, a single dose of Compound A SDD (40, 60, or 80 mg) was administered orally (4×10 mg SDD tablets, 6×10 mg SDD tablets, or 8×10 mg SDD tablets). There was a washout period of at least 10 days between each dose of Compound A.

For Period 1: Subjects were required to fast overnight (≥10 hr) on Day −1. On Day 1, they were given a standard meal 1 hr after administration of 40 mg Compound A (4×10 mg SDD tablets).

For Period 2: Subjects were required to fast overnight (≥10 hr) on Day 10. On Day 11, they were given a standard meal 1 hr or 2 hr after administration of 80 Compound A (8×10 mg SDD tablets). The timing of the meal (1 hr or 2 hr post administration of Compound A) was dependent on the mean AUC₀₋₂₄ determined in Period 1.

For Period 3: Subjects were required to fast overnight (≥10 hr) on Day 20. On Day 21, they were given a standard meal 1 hr or 4 hr after administration of 60 or 80 mg Compound A (6×10 mg SDD tablets or 8×10 mg SDD tablets). The dose and timing of the standard meal was dependent on the mean AUC₀₋₂₄ determined in Period 2.

The final study visit occurred on Day 29. Subjects were not allowed to perform strenuous exercise of >30 min/day, 3 days prior to Day −1 and throughout the study. PK and safety assessments including adverse event (AE) monitoring, clinical laboratory tests, vital sign measurements, 12-lead ECGs, and physical examinations were conducted at scheduled times throughout the study.

### Cohort 4:

Cohort 4 consisted of 3 periods as follows:

In Period 1, fasted subjects were given a standard meal 1 hour after administration of 40 mg (4×10 mg) paltusotine SDD tablets.

In Period 2, fasted subjects were given a standard meal 1 hour after administration of 80 mg (8×10 mg) paltusotine SDD tablets.

In Period 3, fasted subjects were given a standard meal 4 hours after administration of 80 mg (8×10 mg) paltusotine SDD tablets.

### Cohort 5:

The cohort consisted of three periods. The PK of 60 mg paltusotine administered as 3×20 mg SDD tablets was assessed using the following criteria: effect of a low-fat meal on the PK of paltusotine (Period 1 and Period 3) and effect of the PPI lansoprazole on the PK of paltusotine (Period 2) as follows:

In Period 1, fasted subjects were given a low-fat meal 1 hour after administration of 60 mg (3×20 mg) paltusotine SDD tablets. Low-fat meal: 400-500 calories such that 25% of the calories are derived from fat (11-14 grams).

In Period 2, subjects were administered lansoprazole (15 mg BID for 3 days, taken orally at least 30 min prior to a meal). On the fourth day, fasted subjects were administered the last dose of lansoprazole (15 mg) 60 min prior to administration of 60 mg paltusotine (3×20 mg SDD tablets). Subjects continued to fast for 1 hour after paltusotine administration, after which they were provided a low-fat meal. Subjects remained fasted until 4 hours following dose administration.

In Period 3, fasted subjects ingested a low-fat meal within 30 minutes. Upon completion of ingestion of the meal (but no more than 30 min after the start of the meal), subjects were dosed with 60 mg paltusotine (3×20 mg SDD tablets). Subjects remained fasted until 4 hours following dose administration

### Study Population:

Up to 36 healthy male or female subjects, between the ages of 18 to 55 years, inclusive, were enrolled. For Cohort 2 only, male and female subjects 18 to 65 years of age, inclusive, at the time of screening.

### Inclusion Criteria

Each subject had to meet all of the following inclusion criteria to be enrolled in the study: Male and female subjects 18 to 55 years of age, inclusive, at the time of screening. For cohort 2 only, male and female subjects 18 to 65 years of age, inclusive, at the time of screening. Body mass index (BMI) of 18 to 30 kg/m², inclusive. Willing to refrain from strenuous, unaccustomed exercise and sports, defined as greater than 30 minutes per day, 3 days prior to Day −1 and throughout the study. If the subject was a heterosexual or bisexual female, she had to be of non-childbearing potential OR must agree to use a highly effective or two clinically acceptable methods of contraception.

### Exclusion Criteria

A healthy subject meeting any of the following criteria was to be excluded from the study: Prior treatment with Compound A. Any uncontrolled or active major systemic disease which makes study participation unsafe or could interfere with evaluation of the endpoints of the study. History or presence of malignancy except adequately treated basal cell or squamous cell carcinomas of the skin within the past 5 years. Active acute or chronic infection. Use of any investigational drug within the past 60 days or 5 half-lives, whichever is longer, prior to the first dosing of study drug. Use of tobacco and/or nicotine-containing products, recreational drugs, or alcohol for 48 hr prior to admission and agreement to refrain from use throughout the study. History of or current alcohol abuse and/or other drug addiction <1 year prior to screening. Used any prescription or over-the-counter (OTC) medication or alternative medicinal products within 14 days of Day −1. Use of caffeine-containing beverages or food for 48 hr prior to Day −1 and for 48 hr prior to each check-in day for all subsequent periods. Have ingested foods containing poppy seeds within 7 days before screening until completion of the study assessments. Taking moderate or strong CYP3A4 inhibitors or inducers. Strenuous exercise for >30 min/day, 3 days prior to Day −1 and throughout the study. Had a blood loss ≥500 mL or donated blood within 3 months prior to admission. Have amylase and/or lipase levels >2×ULN, alanine aminotransferase (ALT) and/or aspartate aminotransferase (AST) >2×ULN, total bilirubin >1.5×ULN (except in the case of known Gilbert's syndrome), and/or serum creatinine above the upper limit of normal. History of hypersensitivity reactions to any excipients in the study drug. Tested positive at screening for human immunodeficiency virus (HIV), hepatitis B surface antigen (HBsAg), or hepatitis C antibody (HCV-Ab), or has a history of a positive result. Female subjects who have a positive serum pregnancy test or are breastfeeding. For Cohort 1 only, subjects that are classified as CYP2C19 poor metabolizers or ultra-rapid metabolizers.

### Test Product, Dose, and Mode of Administration:

10 mg tablets (SDD). Multiple tablets were swallowed with water depending on dose specified for a given period/cohort.

20 mg tablets (SDD). Multiple tablets were swallowed with water depending on dose specified for a given period/cohort.

### Reference Therapy, Dose, and Mode of Administration:

10 mg HMG capsule formulation served as the reference formulation. Multiple capsules were swallowed with water depending on dose specified for a given period/cohort.

### Plasma Pharmacokinetic Parameters:

Blood PK samples were collected to evaluate Compound A plasma concentrations.

PK parameters were calculated for Compound A and the following are shown in tables below: Area under the plasma concentration curve from 0 to 24 hours (AUC₀₋₂₄); Maximum plasma concentration (Cₘₐₓ); Time to achieve maximum plasma concentration (Tₘₐₓ);

### Results

Results from this clinical trial showed that the co-administration of proton pump inhibitors only had a small effect on the pharmacokinetics observed with the administration of the SDD tablets, shorter fasting times are realized with the SDD tablets and the SDD tablets provide better dose proportional pharmacokinetics.

Results from Cohort 1 is presented in Table 17.

TABLE 17
Results from Cohort 1

| | | |
|---|---|---|
| Mean | SD | CV % |

Period 1 (SDD 20 mg + PPI)

| Tₘₐₓ (hr) | 2 (1.5-3) | | |
|---|---|---|---|
| Cₘₐₓ (ng/mL) | 92.5 | 21.6 | 23.3 |
| AUC₀₋₂₄ (hr*ng/mL) | 891 | 240 | 26.9 |

Period 2 (SDD 20 mg alone)

| Tₘₐₓ (hr) | 3 (1.5-6) | | |
|---|---|---|---|
| Cₘₐₓ (ng/mL) | 114 | 40.6 | 35.5 |
| AUC₀₋₂₄ (hr*ng/mL) | 1140 | 337 | 29.5 |

Period 3 (SDD 20 mg + food)

| Tₘₐₓ (hr) | 1.8 (1.3-4) | | |
|---|---|---|---|
| Cₘₐₓ (ng/mL) | 16.7 | 6.82 | 40.9 |
| AUC₀₋₂₄ (hr*ng/mL) | 155 | 46.3 | 29.9 |

Period 4 (SDD 60 mg)

| Tₘₐₓ (hr) | 3 (1-6) | | |
|---|---|---|---|
| Cₘₐₓ (ng/mL) | 305 | 139 | 45.4 |
| AUC₀₋₂₄ (hr*ng/mL) | 2900 | 1240 | 42.6 |

P4/P2 Ratio

| | |
|---|---|
| Cₘₐₓ | 3.0 (1.7-3.4) |
| AUC₀₋₂₄ | 2.8 (1.6-3.7) |

Median and range are reported for Tₘₐₓ. Mean and range are reported for P4/P2 ratios. All dose administered with overnight fast and 2 h post dose fast. Food: high fat meal.

Cohort 1 (SDD 10 mg×2 under different conditions): the observed exposures with and without PPI are fairly comparable. Cohort 1 (SDD 10 mg×2 vs. 10 mg×6): Relatively dose proportional increases in exposures were observed.

SDD tablets exhibited dose proportional increase in total systemic exposure (AUC) up to a dose of 80 mg.

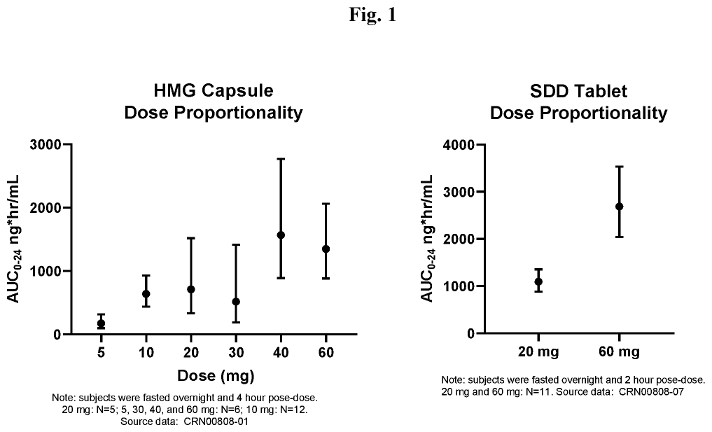

In comparison to the SDD tablets, relatively dose proportional increases in exposures were not observed with the HMG capsules. See FIG. 1. Dose proportionality data for the HMG capsules obtained from a previous clinical study is presented in Table 18.

TABLE 18
Comparative data: Dose Proportionality Observed for HMG Capsule
Formulations after single dose (4 h post dose fast).

| Parameter | | 5 mg | 10 mg | 20 mg | 30 mg | 40 mg | 60 mg |
|---|---|---|---|---|---|---|---|
| (Mean) | Day | (n = 5) | (n = 6) | (n = 5) | (n = 5) | (n = 6) | (n = 6) |
| Tmax (hr) | 1 | 1.2 ± 0.11 | 1.8 ± 0.94 | 2.4 ± 0.82 | 1.4 ± 0.91 | 3.4 ± 1.1 | 3.0 ± 1.2 |
| Cₘₐₓ (ng/mL) | 1 | 16.8 ± 7.22 | 78.7 ± 45.3 | 88.7 ± 43.3 | 78.2 ± 60.7 | 185 ± 118 | 154 ± 77.6 |
| AUC₀₋₂₄ | 1 | 167 ± 73.4 | 661 ± 340 | 811 ± 409 | 578 ± 411 | 1770 ± 888 | 1450 ± 656 |
| (ng hr/mL) | | | | | | | |

Data shown are mean ± standard deviation

Results from Cohort 2 is presented in Table 19.

TABLE 19
Results from Cohort 2

| | | |
|---|---|---|
| Mean | SD | CV % |

Period 1 (HMG 2 h fast)

| Tₘₐₓ (hr) | 2 (0.75-3) | | |
|---|---|---|---|
| Cₘₐₓ (ng/mL) | 123 | 54.4 | 44.4 |
| AUC₀₋₂₄ (hr*ng/mL) | 1060 | 462 | 43.6 |

Period 2 (SDD 2 h fast)

| Tₘₐₓ (hr) | 2 (1-3) | | |
|---|---|---|---|
| Cₘₐₓ (ng/mL) | 97.2 | 32.8 | 33.7 |
| AUC₀₋₂₄ (hr*ng/mL) | 877 | 320 | 36.5 |

Period 3 (SDD 1 h fast)

| Tₘₐₓ (hr) | 1.4 (0.75-4) | | |
|---|---|---|---|
| Cₘₐₓ (ng/mL) | 89.3 | 37.0 | 41.4 |
| AUC₀₋₂₄ (hr*ng/mL) | 721 | 319 | 44.2 |

Period 4 (SDD 30 min fast)

| Tₘₐₓ (hr) | 1.3 (0.75-4) | | |
|---|---|---|---|
| Cₘₐₓ (ng/mL) | 81.3 | 26.8 | 32.9 |
| AUC₀₋₂₄ (hr*ng/mL) | 630 | 212 | 33.6 |

P3/P2 Ratio (%)

| | |
|---|---|
| Cₘₐₓ | 92% |
| AUC₀₋₂₄ | 82% |

All dose administered with overnight fast.

Cohort 2 (SDD 10 mg×2 vs. HMG and under different post-dose fasting durations): SDD tablets did not appear to have better exposures than HMG capsules and the two formulations were relatively comparable. For the SDD tablets the AUC₀₋₂₄ (a measure of extent of absorption) after a 1 hr post dose fast was decreased to 82% of that observed with 2 hr post-dose fast, which is a relatively small decrease in exposure.

In comparison to the performance of the SDD tablets under different post-dose fasting duration scenarios, the HMG capsules performed poorly under different post-dose fasting duration scenarios in a previously completed clinical study. Pharmacokinetic data obtained after administering a 20 mg dose (10 mg HMG capsule×2) in 12 subjects (N=4 male, N=8 Female) is presented in Table 20.

TABLE 20
Comparative Data: HMG capsule Performance
Under Different Post-Dose Fasting Durations

| Post-Dose | | | | | |
|---|---|---|---|---|---|
| Fasting Duration | Tₘₐₓ (Hr) | Cₘₐₓ (ng/mL) | | AUC₀₋₂₄ (Hr*ng/mL) | |
| 4 hour | 0.75-6 | 134 | (48.9) | 1280 | (344) |
| 2 hour | 0.75-3 | 104 | (35.5) | 930 | (293) |
| 1 hour | 0.75-3 | 92.2 | (30.6) | 654 | (244) |

| | | | |
|---|---|---|---|
| Ratio (%): 2 hr/1 hr | — | 89% | 70% |

Cmax and AUC₀₋₂₄ data show are mean (standard deviation). Range is shown for Tmax.

With the HMG capsule formulations, approximately 30% loss in extent of absorption was noted with a 1-hour post-dose fasting vs. a 2-hour post-dose fast.

HMG capsules with a 2h fast was evaluated in Phase 2 clinical studies. A 1 hour fast is more desirable than a 2 hour fast. Only 18% loss of AUC (0-24) was observed with 1 hour fast compared to a 2 hour fast. SDD 1h fast will be utilized for Phase 3. Importantly, SDD tablets appeared to have better dose proportionality than HMG capsules, allowing for 3.0× dose (i.e., 60 mg) to be administered in Phase 3 clinical studies.

Results from Cohort 5 is presented in Table 21.

TABLE 21
Results from Cohort 5 (PPI)

| | | | Cₘₐₓ | AUC_{0inf} | Tₘₐₓ |
|---|---|---|---|---|---|
| Period | Treatment | N | (ng/mL) | (hr*ng/mL) | (hr) |
| 1 | 60 mg | 12 | 250 | 4050 | 2.0 |
| | | | (47.5%) | (46.4%) | [1.0-4.0] |
| 2 | 60 mg + PPI | 12 | 153 | 2450 | 2.0 |
| | | | (48.8%) | (58.2%) | [1.3-6.0] |

Mean (CV %), except Tₘₐₓ where median [range] are reported.

The exposures (Cₘₐₓ and AUC) of Compound A appeared to be reduced by ˜40% when administered with PPI.

Additional results from Cohort 5 is presented in Table 22.

TABLE 22
Results from Cohort 5 (Low-fat meal)

| | | | Cₘₐₓ | AUC_{0-inf} | Tₘₐₓ |
|---|---|---|---|---|---|
| Period | Treatment | N | (ng/mL) | (hr*ng/mL) | (hr) |
| 1 | 60 mg | 11 | 256 | 4140 | 2.0 |
| | | | (47.8%) | (46.8%) | [1.0-4.0] |
| 3 | 60 mg + Low-fat | 11 | 104 | 1650 | 2.0 |
| | meal | | (63.9%) | (55.1%) | [1.3-4.0] |

Mean (CV %), except Tₘₐₓ where median [range] are reported.

The exposures (Cₘₐₓ and AUC) of Compound A appeared to be reduced by ˜60% when administered with low-fat meal, compared to >80% reduction when administered with high-fat meal.

### Example 6: A Phase 2, Study to Evaluate Compound A in Patients with Acromegaly Treated With Somatostatin Analogue Based Treatment Regimens (ACROBAT Edge)

An open label exploratory study designed to evaluate the safety, efficacy, and pharmacokinetics of Compound A (also known as paltusotine) in subjects with acromegaly that are treated with somatostatin analogue (SSA) based treatment regimens.

### Study Design:

Intervention Model: Single Group Assignment.
Masking: None (Open Label).
Primary Purpose: Treatment.

### Test Product, Dose, and Mode of Administration:

10 mg HMG capsule formulation. Multiple capsules were swallowed with water depending on dose specified for a given period/cohort.

### Outcome Measures

Primary Outcome Measures: Change from baseline (mean of Screening values) in insulin-like growth factor-1 (IGF-1) level [Time Frame: 13 Weeks].

Secondary Outcome Measures: 1) Proportion of subjects with their last IGF-1 measurement ≤upper limit of normal (ULN) [Time Frame: 13 Weeks]. 2) Proportion of subjects with their last IGF-1 measurements ≤1.5×ULN [Time Frame: 13 Weeks].

### Eligibility Criteria

### Inclusion Criteria

Male and female subjects 18 to 70 years of age. Confirmed diagnosis of acromegaly with either a partial or complete response to protocol defined somatostatin analogue therapy regimens. Females must be non-pregnant and non-lactating, and either surgically sterile, post-menopausal, or using effective method(s) of birth control. Willing to provide signed informed consent.

### Exclusion Criteria

Treatment naïve acromegaly subjects. Prior treatment with paltusotine. Pituitary surgery within 6 months prior to Screening. Subjects receiving radiation therapy may be eligible with some restrictions. History or presence of malignancy except adequately treated basal cell and squamous cell carcinomas of the skin within the past 5 years. Use of any investigational drug within the past 30 days or 5 half-lives, whichever is longer. Positive test at Screening for HIV, hepatitis B surface antigen (HBsAg) or hepatitis C antibody (HCV-Ab) or has a history of a positive result. History of alcohol or substance abuse in the past 12 months. Any condition that in the opinion of the investigator would jeopardize the subject's appropriate participation in this study. Cardiovascular conditions or medications associated with prolonged QT or those which predispose subjects to heart rhythm abnormalities. Subjects with symptomatic cholelithiasis. Subjects with clinically significant abnormal findings during the Screening Period, and any other medical condition(s) or laboratory findings that, in the opinion of the Investigator, might jeopardize the subject's safety or ability to complete the study. Subjects taking octreotide LAR at a dose higher than 40 mg, or lanreotide depot at a dose higher than 120 mg, or pasireotide LAR at a dose higher than 60 mg. Subjects who usually take octreotide LAR or lanreotide depot less frequently than every 4 weeks (e.g. every 6 weeks or 8 weeks).

### Example 7: A Phase 2, Study to Evaluate the Safety and Efficacy of Compound A for the Treatment of Acromegaly (ACROBAT Evolve)

A Phase 2 double-blind, placebo-controlled, randomized withdrawal study is designed to evaluate the safety, efficacy, and pharmacokinetics of Compound A in subjects with acromegaly that are responders to octreotide LAR or lanreotide depot.

### Study Design:

Allocation: Randomized.
Intervention Model: Parallel Assignment.
Masking: Triple (Participant, Care Provider, Investigator).
Primary Purpose: Treatment.

### Test Product, Dose, and Mode of Administration:

10 mg HMG capsule formulation. Multiple capsules were swallowed with water depending on dose specified for a given period/cohort.

### Outcome Measures

Primary Outcome Measures: Proportion of subjects who meet responder criteria (based on the mean of two consecutive insulin-like growth factor-1 [IGF-1] measurements ≤upper limit of normal [ULN]) (Time Frame: 13 Weeks).

Secondary Outcome Measures: 1) Change in IGF-1 levels [Time Frame: From Week 10 to Week 13]. 2) Change in growth hormone (GH) levels [Time Frame: From Week 8 to Week 13]. 3) Change in patient assessed symptoms of acromegaly [Time Frame: From Week 10 to Week 13]. Total score computed by adding each of the individual acromegaly symptom intensities (headache pain, joint pain, sweating, fatigue, weakness in legs, swelling, numbness or tingling).

### Eligibility Criteria

### Inclusion Criteria

Male and female subjects 18 to 70 years of age. Confirmed diagnosis of acromegaly that is controlled on stable doses of octreotide LAR or lanreotide depot. Females must be non-pregnant and non-lactating, and either surgically sterile, post-menopausal, or using effective method(s) of birth control. Willing to provide signed informed consent.

### Exclusion Criteria

Treatment naïve acromegaly subjects. Prior treatment with Compound A. Pituitary surgery within 6 months prior to Screening or radiation therapy at any time prior to the study entry. Pituitary radiation therapy (within 3 to 4 years or more than 4 years prior to study entry) with recently documented elevated IGF-1 may be eligible. History or presence of malignancy except adequately treated basal cell and squamous cell carcinomas of the skin within the past 5 years. Use of any investigational drug within the past 30 days or 5 half-lives, whichever is longer. Positive test at Screening for HIV, hepatitis B surface antigen (HBsAg) or hepatitis C antibody (HCV-Ab) or has a history of a positive result. History of alcohol or substance abuse in the past 12 months. Any condition that in the opinion of the investigator would jeopardize the subject's appropriate participation in this study. Cardiovascular conditions or medications associated with prolonged QT or those which predispose subjects to heart rhythm abnormalities. Subjects with symptomatic cholelithiasis. Subjects with clinically significant abnormal findings during the Screening Period, and any other medical condition(s) or laboratory findings that, in the opinion of the Investigator, might jeopardize the subject's safety or ability to complete the study. Subjects who have been taking the following prior medications: pegvisomant (within the last 3 months), dopamine agonists (within the last 3 months) and pasireotide LAR (within the last 6 months). Subjects taking octreotide LAR at a dose higher than 40 mg or lanreotide depot at a dose higher than 120 mg. Subjects who usually take octreotide LAR or lanreotide depot less frequently than every 4 weeks (e.g. every 6 weeks or 8 weeks).

Results from the Phase 2 Trials

25 patients were enrolled: patients treated with octreotide or lanreotide & baseline IGF-1 (×ULN): >1 and <2.5.

Prespecified Primary Analysis Population: patients treated with SRL (octreotide or lanreotide) with elevated IGF-1 at baseline—representing the majority of patients in clinical practice. The primary hypothesis was that the group would show no change in the median IGF-1 at Week 13 versus baseline.

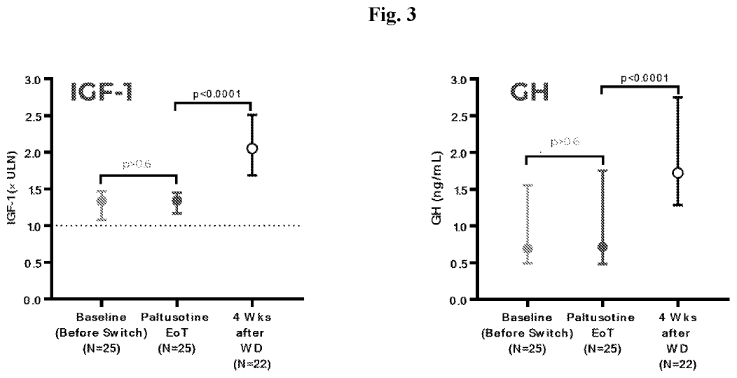

As shown in FIG. 3, paltusotine maintained IGF-1 and GH levels after switching from injected SRL peptide depots (data presented are median (Interquartile Range [IQR]: 25th percentile, 75th percentile) EoT=End of Treatment defined as Week 13 (Visit 14) or last on treatment value carried forward (LOCF). Wks after WD is defined as Week 17 or result at least 22 days after last dose. Note: p-values are based on non-parametric Wilcoxon Sign Rank test of whether the median change is different from zero). IGF-1 levels after 13 weeks of paltusotine treatment did not significantly change from baseline in patients previously treated with injected SRL depots. Rise in IGF-1 after withdrawal (within 2 weeks) which characterized the magnitude of therapeutic activity for oral paltusotine. GH levels after 13 weeks of paltusotine treatment did not significantly change from baseline levels when patients were previously treated with injected SRL depots. Rise in GH after withdrawal characterized the magnitude of therapeutic activity of oral paltusotine.

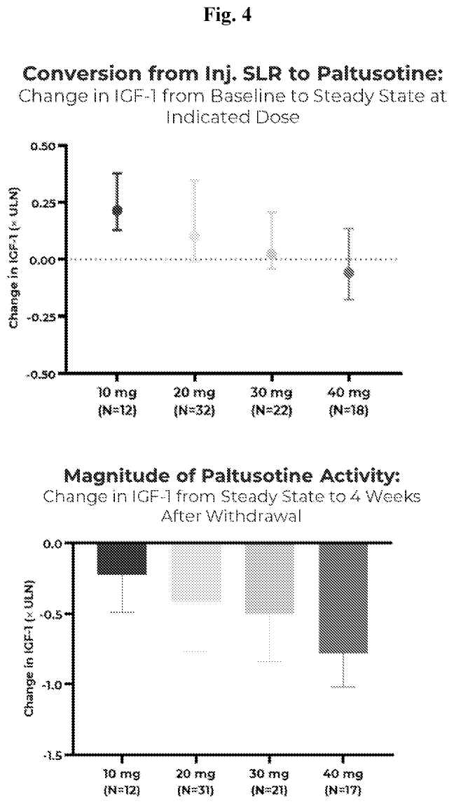

FIG. 4 illustrates the evidence of a dose response observed from the Acrobat and Evolve trials. However, the HMG capsule formulations that were used lacked dose proportional pharmacokinetics above 40 mg.

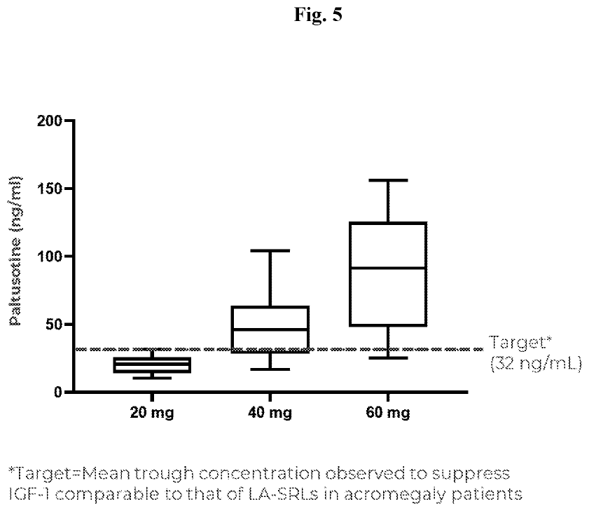

As shown in FIG. 5, administration of the SDD tablets, 40 mg/day and 60 mg/day, is projected to yield trough concentrations consistently in therapeutic range.

In some embodiments, a trough concentration of paltusotine required to provide therapeutic effect comparable to that of long acting SRLs in acromegaly patients is greater than 20 ng/mL, greater than 25 ng/mL, greater than 30 ng/mL, greater than 35 ng/mL, or greater than 40 ng/mL. In some embodiments, a trough concentration of paltusotine required to provide therapeutic effect comparable to that of long acting SRLs in acromegaly patients is at least about 20 ng/mL, at least about 25 ng/mL, at least about 30 ng/ml, at least about 35 ng/mL, at least about 40 ng/mL, or at least about 50 ng/mL. In some embodiments, paltusotine is administered at doses sufficient to provide a trough concentration of paltusotine that is at least about 20 ng/ml, at least about 21 ng/ml, at least about 22 ng/ml, at least about 23 ng/ml, at least about 24 ng/mL, at least about 25 ng/ml, at least about 26 ng/ml, at least about 27 ng/mL, at least about 28 ng/mL, at least about 29 ng/ml, at least about 30 ng/ml, at least about 31 ng/ml, at least about 32 ng/ml, at least about 33 ng/ml, at least about 34 ng/ml, at least about 35 ng/mL, at least about 36 ng/ml, at least about 37 ng/ml, at least about 38 ng/ml, at least about 39 ng/ml, or at least about 40 ng/mL. In some embodiments, paltusotine is administered at doses sufficient to provide a trough concentration of paltusotine that is at least about 32 ng/ml.

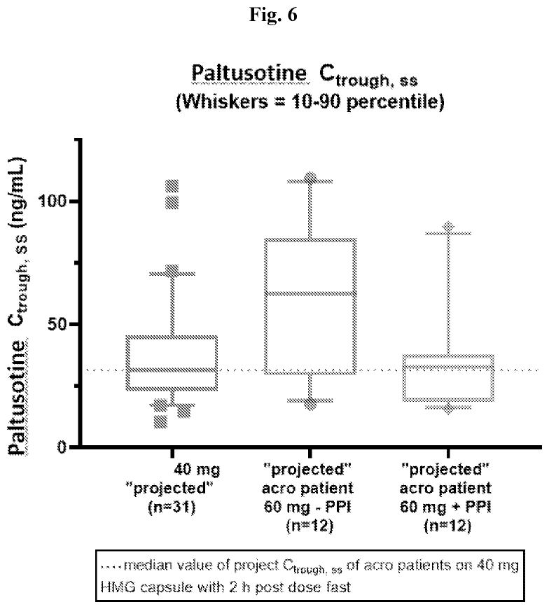

As shown in FIG. 6, the median “projected” steady state trough concentrations of acromegaly patients taking PPI who are on 60 mg SDD (1 h post dose fast) is similar to the median steady state trough concentrations of acromegaly patients on 40 mg HMG (2 h post dose fast).

### Example 8: A Phase 2, Randomized, Parallel Group Study to Evaluate the Safety, Pharmacokinetics, and Dose Response of Paltusotine Treatment in Subjects with Carcinoid Syndrome

The purpose of this study is to evaluate the safety, pharmacokinetic (PK), and dose response of paltusotine treatment in subjects with carcinoid syndrome.

### Overall Design

This is a Phase 2, randomized, open-label, parallel-group, multi-center study. The study includes a Screening period of up to 12 weeks. After completion of Screening, subjects will be randomly assigned to 40 mg QD vs. 80 mg QD open-label dose groups for up to 8 weeks, referred to as the Randomized Treatment Phase.

Subjects who complete the Randomized Treatment Phase may be eligible to enter the Open-Label Extension (OLE) Phase of the study in which subjects will receive paltusotine for 50 weeks. The total duration of paltusotine treatment is up to 58 weeks or up to 15 months.

### Duration and Intervention Groups

The study comprises of 2 phases: Randomized Treatment Phase and OLE Phase. Subjects who participate in both the phases will complete the study in approximately 58 to 70 weeks or 14 to 16 months. The study will consist of: Screening period: Up to 12 weeks; Randomized Treatment Phase: 8 weeks; Randomized 1:1 to 40 mg or 80 mg for 8 weeks; OLE Phase: 50 weeks.

### Study Drug

Paltusotine will be provided as 20 mg tablets. During the Randomized Treatment Phase, those assigned to 40 mg will take two 20 mg tablets daily and those assigned to 80 mg will take four 20 mg tablets daily. Those who require 120 mg will take six 20 mg tablets daily.

### Screening

The Screening Period will vary from 2 to 12 weeks, depending on when subjects meet the qualifying criteria for randomization as described below.

Subjects who are naïve to SRLs and actively symptomatic (average of ≥4 bowel movements (BM)/day (on 3 or more consecutive days) or >2 flushing episodes per day in at least 2 days over a period of 2 weeks) or who are currently symptomatically controlled (average <4 bowel movements [BMs]/day with ≤5 BMs on any single day and average ≤2 flushing episodes/day over a 2-week period) while treated with lanreotide, octreotide LAR, or short-acting octreotide (immediate release octreotide injection or oral octreotide) and who are willing to wash out of their medication will be eligible for Screening.

The completion of an electronic symptom diary will continue daily throughout Screening, and through Week 12 of the study (through Randomized Treatment Phase and 4 weeks into OLE) and then selected time periods in the OLE Phase. Stool consistency according to the Bristol scale and abdominal pain according to a NRS will also be recorded in the electronic symptom diary. Some endpoints use Baseline defined from the Screening Period which is defined as the last 7 days prior to start of randomized treatment. Otherwise Baseline is defined as the last value prior to start of randomized treatment.

Subjects Naïve to SRLs: For subjects naïve to SRLs, 2 Screening visits (S1 and S2) will be scheduled in the Screening Period. After completing the initial eligibility assessment in S1, a 2 week assessment of untreated symptoms (BMs and flushing episodes) frequency will be initiated. At the S1 visit, plasma 5 HIAA levels will be assessed. When the plasma 5-HIAA result is ≥2× the upper limit of normal (ULN), and the 2 week assessment is complete, demonstrating the subject meets symptomatic qualifying criteria (average of ≥4 BM/day OR >2 flushing episodes per day in at least 2 days over the period of 2 weeks), the Day 1 randomization visit should be scheduled.

### Subjects Controlled Symptomatically Using Pretrial SRLs

Lanreotide or Octreotide LAR: For subjects using pretrial lanreotide or octreotide LAR, 2 Screening visits (S1 and S2) will be scheduled in the Screening Period. Screening Visit 1 (S1) should be scheduled so that the interval between the last pretrial injection of lanreotide or octreotide LAR and S2 is not longer than the usual interval between injections for the subject. S2 will occur up to 4 weeks after the last pretrial lanreotide or octreotide LAR injection. These subjects will not receive lanreotide or octreotide LAR after the Informed Consent is given at S1. Completion of the electronic symptom diary should begin within 1 day of S1.

At the S2 Visit, symptom control from pretrial lanreotide or octreotide LAR will be assessed. Subjects who are symptomatically controlled (having an average of <4 BM/day with ≤5 BMs on any single day and an average of ≤2 flushing episodes/day) over the 2-week period (on 3 or more consecutive days) between S1 and S2 will have their plasma 5HIAA level assessed and continue in the Screening Period for up to 10 weeks after S2. During this time, it will be necessary for the subject to meet symptomatic qualifying criteria (an increase from the period documented between S1 and S2 of ≥2 BMs above the daily treated average OR an increase in daily average flushing episodes with ≥3 episodes on at least 1 day during a 7-day period). If the subject does not qualify for the Day 1 randomization visit within 10 weeks of S2, the subject will screen fail.

Short-acting Octreotide: For subjects using regular doses of short-acting octreotide (immediate release octreotide injection or oral octreotide) for the prevention of carcinoid syndrome symptoms pretrial, 2 Screening visits (S1 and S2) will be scheduled in the Screening Period. After S1, a 2 week assessment of treated symptoms (BMs and flushing episodes) frequency will be initiated. The subject should continue pretrial dosing of short-acting octreotide at the most recent pretrial dose and administration frequency during this period. Completion of the electronic symptom diary should begin within 1 day after Informed Consent is given at the S1. The S2 will occur approximately 2 weeks after S1.

At the S2 Visit, symptom control from short-acting octreotide will be assessed. Subjects having an average of <4 BM/day with ≤5 BMs on any single day and an average of ≤2 flushing episodes/day over the 2-week period between S1 and S2 will have sampling of plasma 5-HIAA and continue in the Screening Period. During the Screening Period, it will be necessary for the subject to demonstrate adequate washout of short-acting octreotide by meeting symptomatic qualifying criteria (an increase from the period documented between S1 and S2 of ≥2 BMs above the daily treated average OR an increase in daily average flushing episodes with ≥3 episodes on at least 1 day, during a 7-day period). If Day 1 cannot be scheduled within 10 weeks of S2, the subject is considered a screen-failure.

Antidiarrheal agents may be used during Screening as recommended by the Investigator. Representative guidelines for recommending oral antidiarrheal agents during the screening period to the subjects are as follows:

TABLE A
Oral Antidiarrheal Agents During the Screening Period

| Regimen | Criteria for Initiating | Dose and Frequency |
|---|---|---|
| Diphenoxylate | As needed for improved symptom | 5 mg ≤4 × daily as needed |
| | control | |
| Loperamide | As needed for improved symptom | 4 mg initiation, 2 mg after each |
| | control | loose bowel movement as needed; |
| | | total daily dose ≤16 mg |

Diphenoxylate or loperamide may be used as needed for improved symptom control at any time during the study except when short-acting octreotide is being used for rescue therapy. If there is a delay in scheduling the Day 1 randomization visit for subjects in Screening who have met the symptomatic criteria qualifying them for randomization, subjects may begin short-acting octreotide. Short-acting octreotide must be stopped no later than 12 hours prior to the randomization visit.

TABLE B
Use of Short-acting Octreotide During the Screening Period.

| Pretrial Treatment | Required to Start Short-acting | Criteria for Stopping |
|---|---|---|
| Status | Octreotide* | Short-acting Octreotide |
| Naïve | ≥4 BM/day for 2 consecutive days or if | Symptom relief for 24 |
| | flushing frequency is ≥3 flushing | hours after initiation of |
| | episodes/day for at least 1 day | short-acting octreotide |
| SRL treated: | Increased bowel movement frequency | Symptom relief for 24 |
| Octreotide LAR | with ≥2 BM/day for 2 consecutive days | hours after initiation of |
| Short-acting | above the screening daily treated average | short-acting octreotide |
| octreotide | measured between S1-S2 or ≥3 flushing | |
| Lanreotide depot | episodes/day for at least 1 day | |

BM = bowel movement; LAR = long-acting release; S1 = Screening Visit 1; S2 = Screening Visit 2.
*Symptoms requiring treatment are defined as diarrhea or flushing episodes of severe intensity, ie, symptoms which prevent normal everyday activities.

### Randomized Treatment Phase

Once all Screening assessments are complete and the subject's eligibility is verified, the subject will be randomized to 40 mg QD vs. 80 mg QD for the 8-week Randomized Treatment Phase. Subjects will be provided on Day 1 with sufficient paltusotine 20 mg tablets for 40 mg QD or 80 mg QD for 14 days and will return to the site on Day 14 to perform PK assessments. Scheduled study visits will occur on Days 28, 42, and 56. A determination at Day 56 (referred as Week 8 in the SOAs) will be made for potential enrollment in the OLE phase.

### Rescue Treatment of Breakthrough Carcinoid Syndrome Symptoms During Randomized Treatment Phase

Diphenoxylate or loperamide may be used as needed for improved symptom control at any time during the study except when short-acting octreotide is being used for rescue therapy. Possible treatment regimens for breakthrough symptoms of carcinoid syndrome occurring during the study are delineated in Table C.

On Day 1, all subjects who have not already received instructions on how to use short-acting octreotide 200 μg up to 3 times daily, at the direction of the investigator, during the Screening Period will receive instructions for rescue therapy when criteria are met (Table D). Short-acting octreotide should not be given for at least 12 hours before biomarker sample collection.

TABLE C
Representative Treatment Regimens for Breakthrough
Symptoms During the Randomized Treatment Phase

| Regimen | Criteria for Initiating | Dose/Frequency |
|---|---|---|
| Diphenoxylate | As needed for improved | 5 mg ≤4 × daily as needed |
| | symptom control | |
| Loperamide | As needed for improved | 4 mg initiation, 2 mg after each |
| | symptom control | loose bowel movement as needed; |
| | | total daily dose ≤16 mg |
| Short-acting octreotide | Table D rescue criteria | 200 μg up to 3 × daily until criteria |
| | | for stopping (Table D) are met |

TABLE D
Short-acting Octreotide Rescue Therapy During the Randomized Treatment Phase

| Pretrial | Criteria for Starting Short-acting | Criteria for Stopping |
|---|---|---|
| Treatment Status | Octreotide* | Short-acting Octreotide* |
| Naïve | After 3 days of starting study drug or a new | Symptom relief for 24 |
| | study drug dose, ≥4 BM/day for 2 | hours after initiation of |
| | consecutive days or if flushing frequency is | short-acting octreotide |
| | ≥3 flushing episodes/day for at least 1 day | |
| SRL treated: | After 3 days of starting study drug or a new | Symptom relief for 24 |
| Octreotide LAR, | study drug dose, increased bowel movement | hours after initiation of |
| Short-acting | frequency with ≥2 BM/day for 2 consecutive | short-acting octreotide |
| octreotide | days above the screening daily treated | |
| Lanreotide | average measured between S1-S2 or ≥3 | |
| | flushing episodes/day for at least 1 day | |

Note:
If the criteria for rescue therapy are met on a second occasion between visits and the subject starts the second course of short-acting octreotide, the subject should stay on short-acting octreotide regularly until the next scheduled visit. BM = bowel movement; LAR = long-acting release; S1 = Screening Visit 1; S2 = Screening Visit 2.
*Symptoms requiring treatment are defined as diarrhea or flushing episodes of severe intensity, ie, symptoms which prevent normal everyday activities.

If the subject experiences carcinoid syndrome symptoms that meet protocol criteria for short-acting octreotide rescue, the respective dose may be increased by 40 mg QD based on symptomatology to a maximum dose of 80 or 120 mg QD once during the first 28 days. No dose increases are allowed after Day 28 through the remaining 4 weeks of the parallel group phase of the study. Table E summarizes the dose adjustments during the randomized, parallel group portion of the study.

TABLE E
Dose Adjustments During Randomized Treatment Phase

| Carcinoid Symptom Criteria | Tolerability Criteria | Dose Adjustment |
|---|---|---|
| On Day 14 or Day 28: no rescue | Acceptable tolerability | No dose change |
| treatment initiated or required | of current dose | |
| during preceding 7 days | | |
| On Day 14 or Day 28: ≥1 rescue | Acceptable tolerability | 40 mg→80 mg or |
| treatment initiated or required | of current dose | 80 mg→120 mg |
| during preceding 7 days | | |
| Any time during parallel group | Unacceptable tolerability | 80 mg→40 mg or |
| study | of 80 or 120 mg doses | 120 mg→80 mg |

### Open-Label Extension Phase

The last dose of the Randomized Treatment Phase will be administered at the research site on Week 8 visit. Subjects completing the 8 weeks Randomized Treatment Phase may begin the OLE Phase of the study if, in the opinion of the Investigator, the subject may benefit from OLE Phase participation. Subjects who do not complete the Randomized Treatment Phase but for whom the Investigator recommends continuation of treatment on paltusotine, may be eligible to participate in the OLE Phase.

For those rolling over into the OLE, the initial OLE dose will be chosen by the Investigator based on the frequency of rescue treatments and study drug tolerability and will start on Day 57 (Table F).

TABLE F
Dose Adjustments during Open-Label Extension Phase

| | | Dose |
|---|---|---|
| Carcinoid Symptom Criteria | Tolerability Criteria | Adjustment |
| On Day 56: no rescue treatments initiated or | Acceptable tolerability of | No dose change |
| required during preceding week | current dose | |
| After Day 56: <2 rescue treatments initiated or | | |
| required during scheduled visit interval | | |
| On Day 56: ≥1 rescue treatment initiated or | Acceptable tolerability of | 40 mg→80 mg |
| required during preceding week | 40 mg | 80 mg→120 mg* |
| After Day 56: ≥2 rescue treatments** initiated | Acceptable tolerability of | |
| or required during scheduled visit interval | 80 mg | |
| At any time | Unacceptable tolerability | 80 mg→40 mg or |
| | of 80 or 120 mg doses | 120 mg→80 mg |

*The initial dose after the 40 mg increase to 80 mg or 80 mg increase to 120 mg should be administered at the clinical research site.
**Second rescue treatment occurs after having met stopping criteria for initial short-acting octreotide rescue (Table D).

### Rescue Treatment of Breakthrough Carcinoid Syndrome Symptoms

Diphenoxylate or loperamide may be used as needed for improved symptom control at any time during the study except when short-acting octreotide is being used for rescue therapy. Possible treatment regimens for breakthrough symptoms of carcinoid syndrome occurring during the study are delineated in Table C. Short-acting octreotide should not be given for at least 12 hours before biomarker sample collection.

If the subject experiences carcinoid syndrome symptoms that meet protocol criteria for short-acting octreotide rescue, the respective dose may be increased by 40 mg QD based on symptomatology to a maximum dose of 80 or 120 mg QD once during the first 28 days. No dose increases are allowed after Day 28 through the remaining 4 weeks of the parallel group phase of the study. Table E summarizes the dose adjustments during the randomized, parallel group portion of the study.

### Study Completion and Early Termination

Study completion and early termination (ET) are each defined separately for the Randomized Treatment Phase and the OLE Phase. Completion of Randomized Treatment Phase and the OLE Phase of the study requires that subject complete the final visit within each phase (End of Study [EOS] Visit for Randomized Treatment Phase and the OLE Phase). Subjects who do not complete the Randomized Treatment Phase may be eligible to participate in the OLE Phase, at the request of the Investigator and after discussion with the Medical Monitor. Subjects discontinuing early from Randomized Treatment Phase or the OLE Phase of the study should be treated with standard treatment as recommended by the Investigator and will have an ET Visit.

### End of Randomized Treatment Phase and End of Treatment

Completion of last scheduled dosing in Randomized Treatment Phase is defined as End of Randomized Treatment Phase (EOR) and completion of last scheduled dosing in OLE Phase is defined as End of Treatment (EOT).

### End of Study

An EOS Visit (follow up visit) will occur to collect the safety data 28 days after the last dose of paltusotine.

An EOS Visit will be 28 days after last dose of Randomized Treatment Phase for those who do not enter the OLE Phase. For subjects completing the OLE Phase, EOS Visit will be at Week 62.

### Objectives and Endpoints of the Study

To evaluate the safety and tolerability of paltusotine at 40, 80, and 120 mg QD doses. Endpoints include the incidence of TEAEs, including serious TEAEs and TEAEs leading to discontinuation; change from Baseline to the EOR in safety parameters: clinical laboratory tests, physical exam findings, vital signs, 12 lead ECG, and 24-hour continuous cardiac monitoring (only for subjects on 120 mg dose)

To assess the PK of 40, 80, and 120 mg paltusotine. Endpoints include the steady state trough levels at each dose at EOR

### Exploratory Efficacy for Randomized Treatment Phase

To derive responder rates for the different dose arms. Endpoints include the proportion of clinical responders by dose during the last week of the Randomized Treatment Phase:
In subjects who meet diarrhea entry criteria only: have less than four mean bowel movements daily; have a >20% reduction in the mean daily number of bowel movements compared with Baseline
In subjects who meet flushing entry criteria only: have a >30% reduction compared with Baseline in the mean daily number of flushing episodes.
In subject who meet both diarrhea and flushing entry criteria: have less than four mean bowel movements daily; have a >20% reduction in the mean daily number of bowel movements compared with Baseline; have any reduction from Baseline in the mean daily number of flushing episodes.

To evaluate the effect of paltusotine treatment on frequency of BMs/day. Endpoints include the change in mean daily BMs: from the Baseline Period of Screening to the last week of the Randomized Treatment Phase.

To evaluate the effect of paltusotine treatment on frequency of flushing episodes/day. Endpoints include the change in mean daily flushing episodes: from the Baseline Period of Screening to the last week of the Randomized Treatment Phase

To evaluate the effect of paltusotine treatment on biochemical markers of carcinoid syndrome. Endpoints include the change from Baseline to EOR in: Plasma 5-HIAA; Plasma Pancreastatin; Serum Chromogranin A; Serum Serotonin. In some subjects, plasma pancreastatin will not be collected and analyzed.

To evaluate the effect of paltusotine treatment on the use of protocol defined rescue with short-acting octreotide injections. Endpoints include the change in use of short-acting octreotide: change in the proportion of days on short-acting octreotide in the Screening Period, after subject has met entry criteria to the proportion of days on short-acting octreotide of the last week of the Randomized Treatment Phase; change in mean daily dose of short-acting octreotide in the Screening Period after subject has met entry criteria to the mean daily dose of short-acting octreotide during the last week of the Randomized Treatment Phase.

To evaluate the effect of paltusotine treatment on incontinence. Endpoints include the change in the mean daily fecal incontinence episodes (defined as accidental passing of bowel movements including solid stools, liquid stools, or mucus): from the Baseline Period of Screening to the last week of the Randomized Treatment Period.

To evaluate the effect of paltusotine treatment on abdominal pain severity. Endpoints include the change in worst abdominal pain in last 24 hours (using a 0 to 10 NRS): from the mean Baseline Period of Screening to the mean in the last week of the Randomized Treatment Phase; from the highest score during Baseline Period in Screening Period to the highest score during the last week of the Randomized Treatment Phase.

To evaluate the effect of paltusotine treatment on stool consistency. Endpoints include the change in “worst” (highest) stool score in last 24 hours (Bristol scale): from the mean Baseline Period of Screening to the mean in the last week of the Randomized Treatment Phase; from the highest score during Baseline Period in Screening Period to the highest score during the last week of the Randomized Treatment Phase.

To evaluate the effect of paltusotine treatment on health-related quality of life: Change from Baseline to EOR in: EORTC QLQ-C30; EORTC QLQ-GI.NET21 scores; EQ-5D-5L; FACT-CSI.

To evaluate subject-perceived carcinoid symptom severity and change. Endpoints include the: change from Baseline to EOR in PGI-S (Patient Global Impression of Status); PGI-C (Patient Global Impression of Change) at EOR.

To evaluate treatment preference. Endpoints include the change in Treatment preference from Baseline to EOR.

To evaluate the effect of paltusotine treatment on urgency to defecate. Endpoints include the change in mean daily urgency episodes (defined as BMs that make subjects rush to the bathroom): from the Baseline Period of Screening to the last week of the Randomized Treatment Phase

### Open-Label Extension (OLE) Phase

To evaluate the safety and tolerability of paltusotine. Endpoints include the incidence of TEAEs, including serious TEAEs and TEAEs leading to discontinuation; change from EOR to EOT in safety parameters: clinical laboratory tests, physical exam findings, vital signs, and 12-lead ECG

To assess the PK of 40, 80, and 120 mg paltusotine. Endpoints include the steady state trough levels at each dose at EOT.

To evaluate the effect of paltusotine on tumor progression. Endpoints include the incidence of NET progression at EOT using 6 month interval imaging assessment while on paltusotine.

To evaluate the persistence of effect of paltusotine. Endpoints include the: a) proportion of clinical responders during the last week of the OLE phase by dose: i) in subjects who meet diarrhea entry criteria only: have less than four mean bowel movements daily, and have a >20% reduction in the mean daily number of bowel movements compared with Baseline. ii) in subjects who meet flushing entry criteria only: have a >30% reduction compared with Baseline in the mean daily number of flushing episodes. iii) in subject who meet both diarrhea and flushing entry criteria: have less than four mean bowel movements daily, and have a >20% reduction in the mean daily number of bowel movements compared with Baseline and have any reduction from Baseline in the mean daily number of flushing episodes. b) Change from the mean of the last week prior to Baseline to the mean of the last week of participation in the OLE Phase in daily BM frequency. c) Change from the mean of the last week prior to Baseline to the mean of the last week of participation in the OLE Phase in daily flushing episode frequency. d) Change from Baseline to EOT in biochemical markers of carcinoid syndrome. e) Change from Baseline to EOT: EORTC QLQ-C30, EORTC QLQ-GI.NET21 scores, EQ-5D-5L, FACT-CSI.

To evaluate the effect of paltusotine treatment on the use of protocol defined rescue with short-acting octreotide injections. Endpoints include the change in days treated with short-acting octreotide: change in proportion of days on short-acting octreotide in last week prior to Baseline compared to the 1 week prior to EOT; change in mean daily dose of octreotide during the last week prior to Baseline to the 1 week prior to EOT.

To evaluate the effect of paltusotine treatment on incontinence. Endpoints include the change in mean daily fecal incontinence episodes (defined as accidental passing of bowel movements including solid stools, liquid stools, or mucus) days: from the last week prior to Baseline to the last week prior to EOT.

To evaluate the effect of paltusotine treatment on abdominal pain severity. Endpoints include the change in worst abdominal pain in last 24 hours (using a 0 to 10 NRS): from the mean in the last week prior to Baseline to the to the last week of OLE; from the highest score during last week prior to Baseline to the to the last week of OLE.

To evaluate the effect of paltusotine treatment on urgency to defecate. Endpoints include the change in mean daily urgency episodes (defined as BMs that make subjects rush to the bathroom): from the last week prior to Baseline to the last week prior to EOT.

### Number of Subjects

Approximately 30 subjects (15 subjects/arm) will be enrolled in the study. Subjects taking proton-pump inhibitors (PPIs) at Screening may contribute up to 6 subjects enrolled in the study.

### Inclusion Criteria

Willing and able to provide written informed consent prior to any study-related procedures. Willing and able to comply with the study procedures as specified in the protocol, including at least 70% compliance with electronic symptom diary for the 2-week period prior to the Day 1 visit.

Male or female subjects ≥18 years of age, at the time of Screening.

Documented carcinoid syndrome requiring medical therapy including at least one historical instance of an elevated biomarker. Eligible subjects fall into one of the following categories:
Naïve to SRLs and actively symptomatic (average of ≥4 BM/day or >2 flushing episodes per day in at least 2 days over a period of 2 weeks)
Subjects currently treated with lanreotide, octreotide LAR, or short-acting octreotide (subcutaneous or oral) who are currently symptomatically controlled (average <4 BM/day with ≤5 BMs on any single day and average ≤2 flushing episodes/day over a 2-week period) and willing to wash out of their medication. The subject must demonstrate symptomatic worsening after washout.

Evaluable documentation of locally advanced or metastatic histopathologically confirmed well-differentiated NET. Tumors must be Grade 1 or Grade 2 (Ki-67 ≤20%, or a mitotic count of ≤20 mitoses per 10 high-power fields, if the Ki-67 index is not available) per the World Health Organization neuroendocrine neoplasm classification (Rindi G, Inzani F. Neuroendocrine neoplasm update: toward universal nomenclature. Endocr Relat Cancer. 2020; 27(6):R211-R218). Grade 3 tumors are not eligible.

| WHO 2017 | Mitoses/10 high power fields (HPF) | Ki-67 Index |
|---|---|---|
| Well-differentiated neuroendocrine | | |
| neoplasms (NENs) | | |
| neuroendocrine tumor (NET) grade 1 | <2 | <3 |
| neuroendocrine tumor (NET) grade 2 | 2-20 | 3-20 |
| neuroendocrine tumor (NET) grade 3 | >20 | >20 |

Absence of tumor progression in the opinion of the Investigator, documented by standard surveillance within the last 6 months before initiation of study drug dosing.

Historical documentation of positive SSTR tumor status by PET or somatostatin receptor scintigraphy.

Plasma 5-HIAA ≥2×ULN during Screening for naïve subjects not washing out of SRLs.

Females who engage in heterosexual intercourse must be of non-childbearing potential, or be postmenopausal with at least 1 year of amenorrhea, or must agree to use a highly effective method of contraception from the beginning of Screening to the last study visit. In addition to these methods of contraception, the male partner should use a condom from the beginning of Screening to the last study visit.

If the subject is male, the subject should agree to use a condom when sexually active or remain abstinent.

### Exclusion Criteria

Medical History and Medications: Diarrhea attributed to any condition(s) other than carcinoid syndrome (including but not limited to fat malabsorption, bile acid malabsorption, short bowel syndrome, pancreatic exocrine insufficiency, infections, VIPoma, Zollinger-Ellison syndrome). Exception to this are subjects with prior cholecystectomy or small bowel resections, provided diarrhea is controlled prior to washout or if naïve to SRLs. Uncontrolled/severe diarrhea associated with significant volume contraction, dehydration, or hypotension. Requires second line treatments (eg, telotristat) for control of carcinoid syndrome symptoms in the opinion of the Investigator. Treatment with tumor-directed therapy <4 weeks before Screening or hepatic embolization, radiotherapy, peptide receptor radionuclide therapy (PRRT), and/or tumor debulking <12 weeks before Screening. Karnofsky performance status <60%. Major surgery within 8 weeks before Screening. Any malignancy except for eligible NET, basal cell or squamous cell skin carcinoma considered clinically cured, or in situ cervical carcinoma. Life expectancy <12 months from Screening. Diabetes mellitus treated with insulin for less than 6 weeks prior to the study entry, or with change in total daily insulin dose by >15% within 6 weeks prior to Screening. Poorly controlled diabetes mellitus defined as having a hemoglobin A1c (HbA1c) ≥8.5% (i.e., ≥69.5 mmol/mol) or estimated HbA1c based on fructosamine if HbA1c is not evaluable (eg, due to hemoglobinopathy). Current use of medications that are strong inducers of cytochrome P450 3A4 (CYP3A4) within 2 weeks prior to Screening because they may reduce systemic exposure to paltusotine. Unable to administer short-acting octreotide (octreotide acetate injection). Known allergy or hypersensitivity to any of the test materials or related compounds. Any other condition, as judged by the Investigator, that would interfere with this study.

Screening Tests and Evaluations: Active COVID-19 confirmed or suspected based on SARS-CoV-2 PCR testing and clinical symptoms. QT interval corrected using Fridericia's formula (QTcF) >480 msec (or QTcF >500 msec in the presence of complete bundle branch block) or PR interval >240 msec during Screening based on a central reading of an average of 3 ECGs each separated in time by approximately 1 minute after the subject has rested quietly in the supine position for at least 10 minutes without significant stimulation (noise, television, etc.).

Other Criteria: Clinically significant concomitant disease including but not limited to cardiovascular disease, estimated glomerular filtration rate <60 mL/min/1.73 m², cirrhosis, baseline AST and/or ALT >2×ULN, and/or total bilirubin >1.5×ULN. Subjects with previously diagnosed Gilbert's syndrome not accompanied by other hepatobiliary disorders and associated with total bilirubin <3.5 mg/dL (<51.3 μmol/L) will be permitted.

### Subject-Reported Assessments

Planned time points for all subject-reported assessments are provided in the SOAs. When these assessments are done at the same visit, the order will be as follows: (1) Electronic Symptom Diary, (2) Patient Global Impression of Severity (PGI-S), (3) Patient Global Impression of Change (PGI-C), (4) EuroQol 5 Dimensions 5 Level (EQ-5D-5L), (5) EORTC QLQ-C30 Quality of Life Questionnaire, (6) EORTC QLQ-GI.NET21 Quality of Life Questionnaire, (7) Functional Assessment of Cancer Therapy-Carcinoid Syndrome Symptom Index (FACT-CSI) (Shaunfield S, Webster K A, Kaiser K, et al. (2021) Development of the Functional Assessment of Cancer Therapy-Carcinoid Syndrome Symptom Index. Neuroendocrinology 111:850-862), and (8) Treatment Preference Questionnaire.

Electronic Symptom Diary: Subjects will be asked to complete the electronic symptom diary, a brief symptom diary, daily at home beginning after the initial screening and continuing throughout Screening, Week 1 through Week 12 (throughout Randomized Treatment Phase and beginning of the OLE Phase), then for the rest of the OLE Phase it should be completed daily for 2 weeks before the Week 18, 24, 36, 48 and 58 visits (or simply during Week 17-18, Week 23-24, Week 35-36, Week 47-48, and Week 57-58). This electronic symptom diary will be used to assess the understanding and compliance of the subject and to measure the Baseline symptom frequencies of bowel movement (BMs) and flushing frequency, episodes of fecal urgency and incontinence, abdominal pain severity, and stool consistency. The Baseline symptoms of stool consistency will be evaluated according to Bristol scale and abdominal pain according to an NRS.

Subjects will be instructed to complete the diary at about the same time, every evening during the periods specified above. Site staff will review subject compliance with self-reported electronic diary completion as outlined in the electronic diary manual.

Subjects will be asked to record use of all breakthrough symptom treatments in the daily electronic symptom diary.

Global Impressions of Change and Severity: The PGI-S assesses the subject's perception of overall disease severity using a 4-point verbal rating scale (no symptoms, mild symptoms, moderate symptoms, and severe symptoms). The PGI-C assesses the subject's perception of change in disease severity using a 7-point verbal rating scale (much better, moderately better, a little better, no change, a little worse. moderately worse, and much worse).

EQ-5D-5L: The EQ-5D-5L (5 severity levels EQ-5D), developed by the European Quality of Life (EuroQoL), is a standardized instrument to be completed by the subject for use as a measure of health outcomes applicable to a wide range of health conditions. It comprises 5 dimensions of health: mobility, ability to self-care, ability to undertake usual activities, pain and discomfort, and anxiety and depression. Based on qualitative and quantitative studies conducted by the EuroQoL Group, there are 5 options (levels) under each domain: ‘no problems’, ‘slight problems’, ‘moderate problems’, ‘severe problems’ and ‘unable to/extreme problems’. The responses to all 5 dimensions, can be converted to a single summary index, utility (range: 0 to 1), by using value sets. Higher index values represent better health states.

EORTC QLQ-C30 Quality of Life Questionnaire: EORTC QLQ-C30 quality of life questionnaire will be used to assess health-related quality of life in subjects with cancer. The questionnaire entails 28 questions to assess health-related quality of life, each question can be answered on a 1 to 4 scale. Lower scores indicate a better quality of life. There are 2 additional questions, 1 to rate subjects' ‘overall health in the past week’ and 1 to rate the subjects' ‘overall quality of life in the past week’. Each of these 2 questions can be answered on a 1 to 7 scale. Higher scores indicate a better quality of life.

EORTC QLQ-GI.NET21 Quality of Life Questionnaire: EORTC QLQ-GI.NET21 quality of life questionnaire will be used to follow up with symptoms in subjects with neuroendocrine carcinoids. There will be around 21 questions divided into the category of ‘during the past week’ and ‘during the past 4 weeks’, which will be used to follow up with symptoms in subjects. Each of the question can be answered on a 1 to 4 scale. Lower scores indicate a better quality of life.

FACT-CSI: The FACT-CSI is a 24-item instrument that can be scored as a single, multidimensional symptom index, consisting of the following domains: disease-related physical symptoms, disease-related emotional symptoms, treatment side effects, and functional well-being. Subjects select a response to each statement on a 5-point Likert scale ranging from 0 (Not at all) to 4 (Very much). Lower total score indicates a better quality of life (Shaunfield S, Webster K A, Kaiser K, et al. (2021) Development of the Functional Assessment of Cancer Therapy-Carcinoid Syndrome Symptom Index. Neuroendocrinology 111:850-862).

Treatment Preference Question: The Treatment Preference Question is a single question regarding the subject's preferred treatment form, i.e., previously used injections, or oral study drug (or no preference).

The examples and embodiments described herein are for illustrative purposes only and various modifications or changes suggested to persons skilled in the art are to be included within the spirit and purview of this application and scope of the appended claims.

What is claimed is:

1. A method of treating carcinoid syndrome in a human comprising orally administering to the human with carcinoid syndrome a daily dose of 3-[4-(4-amino-piperidin-1-yl)-3-(3,5-difluoro-phenyl)-quinolin-6-yl]-2-hydroxy-benzonitrile (Compound A), or a pharmaceutically acceptable salt thereof, that is equivalent to about 40 mg/day to about 160 mg/day of Compound A-monohydrochloride.

2. The method of claim 1, wherein the daily dose of Compound A, or a pharmaceutically acceptable salt thereof comprises an amount equivalent to about 40 mg/day, about 50 mg/day, about 60 mg/day, about 70 mg/day, about 80 mg/day, about 90 mg/day, about 100 mg/day, about 110 mg/day, or about 120 mg/day of Compound A-monohydrochloride.

3. The method of claim 1 or 2, wherein treatment is initiated at a daily dose equivalent to about 40 mg/day or 80 mg/day of Compound A-monohydrochloride.

4. The method of any one of claims 1-3, wherein treating carcinoid syndrome in the human comprises managing the symptoms of carcinoid syndrome.

5. The method of claim 4, wherein the symptoms of carcinoid syndrome comprise the severity of diarrhea, frequency and intensity of flushing episodes, or combinations thereof.

6. The method of claim 5, wherein the severity of diarrhea comprises the number of bowel movements per day, the number of watery stools as measured by Bristol Stool Scale, or both.

7. The method of any one of claims 1-3, wherein treating carcinoid syndrome in the human comprises reducing the frequency of carcinoid syndrome breakthrough symptoms.

8. The method of claim 7, wherein the breakthrough symptoms comprise ≥4 bowel movements (BMs)/day for 2 consecutive days or flushing frequency ≥3 flushing episodes/day for at least 1 day.

9. A method of treating carcinoid syndrome in a human with carcinoid syndrome comprising orally administering to the human with carcinoid syndrome a pharmaceutical composition comprising 3-[4-(4-amino-piperidin-1-yl)-3-(3,5-difluoro-phenyl)-quinolin-6-yl]-2-hydroxy-benzonitrile (Compound A), or a pharmaceutically acceptable salt thereof;
wherein the human with carcinoid syndrome is treatment naïve; or
wherein the human with carcinoid syndrome was previously treated with a somatostatin analog and the treatment with the somatostatin analog is terminated and a sufficient period of time lapses to allow the somatostatin analog to washout of the human; and
wherein treatment is initiated at a daily dose equivalent to at least about 40 mg/day of Compound A-monohydrochloride.

10. The method of claim 9, wherein the human with carcinoid syndrome is treatment naïve to somatostatin analog therapy and actively symptomatic.

11. The method of claim 10, wherein actively symptomatic comprises an average of ≥4 bowel movements (BM)/day or >2 flushing episodes in at least 2 days over a period of 2 weeks.

12. The method of claim 9, wherein the carcinoid syndrome in the human that was previously treated with a somatostatin analog was symptomatically controlled with the somatostatin analog.

13. The method of claim 12, wherein symptomatically controlled comprises an average of <4 bowel movements (BM)/day with ≤5 BMs on any single day and average ≤2 flushing episodes/day over a 2-week period.

14. The method of claim 12 or claim 13, wherein the somatostatin analog is octreotide, lanreotide, or pasireotide.

15. The method of any one of claims 9-14, wherein treatment is initiated at a daily dose equivalent to about 40 mg/day or 80 mg/day of Compound A-monohydrochloride.

16. The method of claim 15, wherein the daily dose is increased by a daily dose amount equivalent to about 40 mg/day Compound A-monohydrochloride if breakthrough symptoms are observed.

17. The method of claim 16, wherein the breakthrough symptoms comprise ≥4 bowel movements (BMs)/day for 2 consecutive days or flushing frequency ≥3 flushing episodes/day for at least 1 day.

18. A method of treating carcinoid syndrome in a human with carcinoid syndrome comprising orally administering to the human with carcinoid syndrome a daily dose of 3-[4-(4-amino-piperidin-1-yl)-3-(3,5-difluoro-phenyl)-quinolin-6-yl]-2-hydroxy-benzonitrile (Compound A), or a pharmaceutically acceptable salt thereof, that is equivalent to about 40 mg/day to about 80 mg/day of Compound A-monohydrochloride, wherein treating carcinoid syndrome comprises managing the symptoms of the carcinoid syndrome.

19. The method of claim 18, wherein managing the symptoms of the carcinoid syndrome comprises reducing the frequency of: daily bowel movements, episodes of diarrhea, episodes of fecal incontinence, cutaneous flushing, or a combination thereof.

20. The method of claim 18 or claim 19, wherein managing the symptoms of the carcinoid syndrome comprises treating diarrhea, flushing episodes, or both in the human.

21. The method of claim 19 or claim 20, wherein the diarrhea is severe diarrhea.

22. The method of any one of claims 18-21, wherein managing the symptoms of the carcinoid syndrome comprises reducing the number of bowel movements (BM) to less than 4 BM/day for 2 consecutive days, reducing the flushing frequency to less than 3 flushing episodes/day for at least 1 day, or both.

23. The method of any one of claims 18-22, wherein the daily dose of Compound A, or a pharmaceutically acceptable salt thereof, is equivalent to about 40 mg or about 80 mg of Compound A-monohydrochloride.

24. The method of claim 23, wherein the daily dose of Compound A, or a pharmaceutically acceptable salt thereof, is increased by an incremental daily dose that is equivalent to about 10 mg/day, about 20 mg/day, about 30 mg/day, or about 40 mg/day if the severity of the diarrhea, flushing episodes or both does not diminish in severity during treatment with the daily dose of Compound A, or a pharmaceutically acceptable salt thereof.

25. The method of claim 23, wherein the daily dose of Compound A, or a pharmaceutically acceptable salt thereof, is increased by an incremental daily dose that is equivalent to about 40 mg/day.

26. The method of claim 23, wherein the daily dose of Compound A, or a pharmaceutically acceptable salt thereof, is increased by an incremental daily dose that is equivalent to about 40 mg/day and treatment is continued at a daily amount of Compound A, or a pharmaceutically acceptable salt thereof, that is equivalent to about 80 mg or about 120 mg of Compound A-monohydrochloride.

27. A method of treating severe diarrhea, flushing episodes or both in a human with carcinoid syndrome comprising orally administering to the human in need thereof an initial daily dose of 3-[4-(4-amino-piperidin-1-yl)-3-(3,5-difluoro-phenyl)-quinolin-6-yl]-2-hydroxy-benzonitrile (Compound A), or a pharmaceutically acceptable salt thereof.

28. The method of claim 27, wherein the initial daily dose of Compound A, or a pharmaceutically acceptable salt thereof, is equivalent to about 40 mg or about 80 mg of Compound A-monohydrochloride.

29. The method of claim 27 or claim 28, wherein the initial daily dose of Compound A, or a pharmaceutically acceptable salt thereof, is increased by an incremental daily dose that is equivalent to about 10 mg/day, about 20 mg/day, about 30 mg/day, or about 40 mg/day if the severity of the severe diarrhea, flushing episodes or both does not diminish in severity during treatment with the initial daily dose of Compound A, or a pharmaceutically acceptable salt thereof.

30. The method of claim 27 or claim 28, wherein the initial daily dose of Compound A, or a pharmaceutically acceptable salt, or solvate thereof, is increased by an incremental daily dose that is equivalent to about 40 mg/day.

31. The method of claim 27 or claim 28, wherein the daily dose of Compound A, or a pharmaceutically acceptable salt thereof, is increased by an incremental daily dose that is equivalent to about 40 mg/day and treatment is continued at a daily amount of Compound A, or a pharmaceutically acceptable salt thereof, that is equivalent to about 80 mg or about 120 mg of Compound A-monohydrochloride

32. The method of any one of claims 27-31, wherein the severity of the severe diarrhea is measured by the number of bowel movements per day, the number of watery stools as measured by Bristol Stool Scale, or both.

33. The method of any one of claims 27-31, wherein the severity of the flushing episodes is assessed by measuring urinary 5-hydroxyindole acetic acid (5-HIAA) levels, plasma serotonin levels, or both.

34. A method of treating carcinoid syndrome in a human with carcinoid syndrome comprising orally administering to the human with carcinoid syndrome a daily dose of 3-[4-(4-amino-piperidin-1-yl)-3-(3,5-difluoro-phenyl)-quinolin-6-yl]-2-hydroxy-benzonitrile (Compound A), or a pharmaceutically acceptable salt thereof, wherein human with carcinoid syndrome was previously treated with a somatostatin analog; and wherein treatment is initiated at a daily dose equivalent to about 40 mg/day of Compound A-monohydrochloride.

35. A method of treating carcinoid syndrome in a human with carcinoid syndrome comprising orally administering to the human with carcinoid syndrome a daily dose of 3-[4-(4-amino-piperidin-1-yl)-3-(3,5-difluoro-phenyl)-quinolin-6-yl]-2-hydroxy-benzonitrile (Compound A), or a pharmaceutically acceptable salt thereof, wherein human with carcinoid syndrome was previously treated with a somatostatin analog; and wherein treatment is initiated at a daily dose equivalent to about 80 mg/day of Compound A-monohydrochloride.

36. The method of claim 34 or claim 35, wherein the human with carcinoid syndrome responded to and tolerated treatment with a somatostatin analog.

37. The method of any one of claims 34-36, wherein carcinoid syndrome symptoms were previously controlled on octreotide or lanreotide therapy.

38. The method of any one of claims 34-37, wherein the human with carcinoid syndrome is refractory to treatment with somatostatin analog, wherein the somatostatin analog is octreotide, lanreotide, or pasireotide.

39. The method of any one of claims 34-38, wherein treating carcinoid syndrome comprises managing the symptoms of the carcinoid syndrome.

40. The method of claim 39, wherein managing the symptoms of the carcinoid syndrome comprises reducing the frequency of: daily bowel movements, episodes of diarrhea, episodes of fecal incontinence, cutaneous flushing, or a combination thereof.

41. The method of claim 39 or claim 40, wherein managing the symptoms of the carcinoid syndrome comprises treating diarrhea, flushing episodes, or both in the human.

42. The method of claim 41, wherein the diarrhea is severe diarrhea.

43. The method of claim 42, wherein the severity of diarrhea comprises the number of bowel movements per day, the number of watery stools as measured by Bristol Stool Scale, or both.

44. The method of any one of claims 39-43, wherein managing the symptoms of the carcinoid syndrome comprises reducing the number of bowel movements (BM) to less than 4 BM/day for 2 consecutive days, reducing the flushing frequency to less than 3 flushing episodes/day for at least 1 day, or both.

45. The method of any one of claims 34-44, wherein the daily dose of Compound A, or a pharmaceutically acceptable salt thereof, is increased by an incremental daily dose that is equivalent to about 10 mg/day, about 20 mg/day, about 30 mg/day, or about 40 mg/day if the severity of the diarrhea, flushing episodes or both does not diminish in severity during treatment with the daily dose of Compound A, or a pharmaceutically acceptable salt thereof.

46. The method of any one of claims 34-44, wherein the daily dose of Compound A, or a pharmaceutically acceptable salt thereof, is increased by an incremental daily dose that is equivalent to about 40 mg/day.

47. The method of any one of claims 34-44, wherein the daily dose of Compound A, or a pharmaceutically acceptable salt thereof, is increased by an incremental daily dose that is equivalent to about 40 mg/day and treatment is continued at a daily amount of Compound A, or a pharmaceutically acceptable salt thereof, that is equivalent to about 80 mg or about 120 mg of Compound A-monohydrochloride.

48. The method of any one of claims 1-47, wherein the serum 5-hydroxy-indoleacetic acid (5-HIAA) concentration in the human prior to treatment with Compound A is greater than about >280 μmol/L.

49. The method of any one of claims 1-48, wherein treatment with Compound A, or a pharmaceutically acceptable salt thereof, is discontinued if the human does not tolerate Compound A, or a pharmaceutically acceptable salt thereof.

50. The method of any one of claims 1-48, wherein the daily dose of Compound A, or a pharmaceutically acceptable salt thereof, is reduced if the human does not tolerate Compound A, or a pharmaceutically acceptable salt thereof.

51. The method of any one of claims 1-48, wherein the daily dose of Compound A, or a pharmaceutically acceptable salt thereof, is reduced by an amount equivalent to about 40 mg of Compound A-monohydrochloride if the human does not tolerate Compound A, or a pharmaceutically acceptable salt thereof

52. The method of any one of claims 1-51, wherein:
Compound A, or a pharmaceutically acceptable salt thereof, is administered in the form of one or more tablets comprising a spray-dried solid dispersion of 3-[4-(4-amino-piperidin-1-yl)-3-(3,5-difluoro-phenyl)-quinolin-6-yl]-2-hydroxy-benzonitrile monohydrochloride thereof;
one or more additional pharmaceutically acceptable ingredients; and
optionally one or more film coating agents.

53. The method of claim 52, wherein the spray-dried solid dispersion comprises:
(a) 3-[4-(4-amino-piperidin-1-yl)-3-(3,5-difluoro-phenyl)-quinolin-6-yl]-2-hydroxy-benzonitrile monohydrochloride thereof; and
(b) a pharmaceutically acceptable polymer;
wherein 3-[4-(4-amino-piperidin-1-yl)-3-(3,5-difluoro-phenyl)-quinolin-6-yl]-2-hydroxy-benzonitrile monohydrochloride thereof, is dispersed in a polymer matrix formed from the pharmaceutically acceptable polymer.

54. The method of claim 52 or claim 53, wherein the one or more pharmaceutically acceptable ingredients are selected from the group consisting of one or more diluents, one or more disintegrants, one or more lubricants, one or more glidants.

55. The method of any one of claims 52-54, wherein the one or more pharmaceutically acceptable ingredients comprise microcrystalline cellulose, mannitol, pregelatinized starch croscarmellose sodium crospovidone, sodium chloride, 1:1 sodium chloride: potassium chloride, colloidal silicon dioxide, and magnesium stearate.

56. The method of any one of claims 52-55, wherein each tablet comprises about 2% by weight to about 20% by weight of 3-[4-(4-amino-piperidin-1-yl)-3-(3,5-difluoro-phenyl)-quinolin-6-yl]-2-hydroxy-benzonitrile monohydrochloride thereof.

57. The method of any one of claims 52-56, wherein each tablet comprises about 10 mg, about 20 mg, about 40 mg, about 60 mg or about 80 mg of 3-[4-(4-amino-piperidin-1-yl)-3-(3,5-difluoro-phenyl)-quinolin-6-yl]-2-hydroxy-benzonitrile monohydrochloride thereof.

58. The method of any one of claims 52-56, wherein each tablet comprises about 10 mg of 3-[4-(4-amino-piperidin-1-yl)-3-(3,5-difluoro-phenyl)-quinolin-6-yl]-2-hydroxy-benzonitrile monohydrochloride thereof.

59. The method of any one of claims 52-56, wherein each tablet comprises about 20 mg of 3-[4-(4-amino-piperidin-1-yl)-3-(3,5-difluoro-phenyl)-quinolin-6-yl]-2-hydroxy-benzonitrile monohydrochloride thereof.

60. The method of any one of claims 52-59, wherein: the one or more tablets are administered at least 30 minutes before a meal.

61. The method of any one of claims 52-59, wherein: the one or more tablets are administered at least 60 minutes before a meal.

62. The method of any one of claims 52-61, wherein: the one or more tablets are administered with a glass of water on an empty stomach at least 30 minutes before a meal.

63. The method of any one of claims 52-62, wherein: the bioavailability of 3-[4-(4-amino-piperidin-1-yl)-3-(3,5-difluoro-phenyl)-quinolin-6-yl]-2-hydroxy-benzonitrile monohydrochloride thereof, is not substantially affected by the coadministration of proton pump inhibitors, histamine H2-receptor antagonists, or antacids.

64. The method of any one of claims 1-63, wherein: Compound A, or a pharmaceutically acceptable salt thereof, is not co-administered with a drug that alters the pH of the upper gastrointestinal (GI) tract.

65. The method of any one of claims 1-64, wherein: if Compound A, or a pharmaceutically acceptable salt thereof, is co-administered with a drug that alters the pH of the upper gastrointestinal (GI) tract, then the daily dose amount of Compound A, or a pharmaceutically acceptable salt thereof, that is administered is increased.

66. The method of claim 65, wherein the daily dose amount of Compound A, or a pharmaceutically acceptable salt thereof, is increased by an amount equivalent to about 10 mg/day, about 20 mg/day, about 30 mg/day, or about 40 mg/day of Compound A-monohydrochloride.

67. The method of any one of claims 64-66, wherein the drug that alters the pH of the upper gastrointestinal (GI) tract comprises proton pump inhibitors, histamine H2-receptor antagonists, or antacids.
